(19) United States
(12) Patent Application Publication
(10) Pub. No.: US 2004/0198783 A1
(43) Pub. Date: Oct. 7, 2004

(54) Targeted bone marrow protection agents

(75) Inventors: Joseph R. Garlich, Westfield, IN (US); Donald L. Durden, Decatur, GA (US); Tim C. Smith, Indianapolis, IN (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Semafore Pharmaceuticals Inc., Indianapolis, IN

(21) Appl. No.: 10/817,622

(22) Filed: Apr. 2, 2004

Related U.S. Application Data
(60) Provisional application No. 60/460,289, filed on Apr. 3, 2003.

(51) Int. Cl. A61K 31/428

(52) U.S. Cl. 514/367

(57) ABSTRACT

The invention provides a method of inhibiting cell death in a mammal. The method comprises administering to a mammal an effective amount of a composition comprising a cell protection factor covalently linked to a bone targeting agent via a linkage that is cleaved under physiological conditions, whereby the cell protection factor is released from the bone targeting agent in vivo to inhibit cell death. The invention further provides a compound comprising a cell protection factor covalently attached to a bone targeting agent via a linker that is cleaved under physiological conditions.

### CROSS-REFERENCE TO RELATED PATENT APPLICATIONS

This patent application is related to U.S. Provisional Patent Application No. 60/460,289, filed Apr. 3, 2003.

### STATEMENT REGARDING FEDERALLY SPONSORED RESEARCH AND DEVELOPMENT

This invention was made in part with Government support under Grant Number 1R43CA96259-01 awarded by the National Cancer Institute. The Government may have certain rights in this invention.

### FIELD OF THE INVENTION

This invention pertains to materials and methods for inhibiting cell death in a mammal.

### BACKGROUND OF THE INVENTION

Programmed cell death, known as apoptosis, is a tightly regulated cellular process which serves, in part, to prevent proliferation of abnormal or damaged cells. A subset of genes responsible for initiating cellular processes leading to apoptosis are classified as “tumor suppressor genes” for their role in preventing tumor formation. For example, the p53 protein is a key player in the cellular stress response mechanism. In response to DNA damage, the tumor suppressor protein, p53, ceases cell division or causes the cell to undergo apoptosis. Accordingly, p53 can stop tumor formation by preventing cells that have incurred malignant mutation from dividing to form a tumor.

The p53 gene is, however, susceptible to damage. Inactive or mutated p53 protein (as well as other tumor suppressor gene products) can contribute to genetic instability and tumor formation. It is thought that roughly half of all cancers (including skin, breast, and colon cancers) possess mutant, inactive p53 genes. Treatment of such cancers, however, is impeded by active p53 protein in normal tissue. Functional p53 protein imparts sensitivity to genotoxic stress, such as that caused by radiation or chemical stress, in normal, healthy tissue. Such sensitivity to chemical or radiation-based therapeutics causes damage to normal tissue, while the therapeutics act on malignant, target tissue. For example, p53-mediated damage to the lymphoid system, the hematopoietic system, intestinal epithelium, and hair follicles contribute to collateral damage associated with cancer therapies, which often limits the maximum tolerated doses of drugs in treatment regimens.

Blocking the toxic side effects in normal tissue associated with cancer therapies would allow development of aggressive cancer treatment regimens. However, preventing the body's natural response to genotoxic stress may, in turn, permit abnormal cell growth. There remains a need in the art for alternative compositions and methods for safely protecting normal tissues from apoptotic damage, in particular apoptotic damage associated with disease treatment. The invention provides such compositions and methods. These and other advantages of the invention, as well as additional inventive features, will be apparent from the description of the invention provided herein.

### BRIEF SUMMARY OF THE INVENTION

The invention provides a method of inhibiting cell death in a mammal. The method comprises administering to a mammal an effective amount of a composition comprising a cell protection factor covalently linked to a bone targeting agent via a linkage that is cleaved under physiological conditions, whereby the cell protection factor is released from the bone targeting agent in vivo to inhibit cell death.

The invention further provides a compound of Formula V:

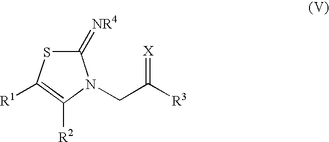

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R⁴ is hydrogen or an C₁-C₆ acyl group when X is Q, or R⁴ is Q when X is a carbonyl or protected carbonyl. X is Q, a carbonyl, or a protected carbonyl. Q is an organic moiety that contains a nucleophilic or electrophilic reacting group and is cleavable under physiological conditions, thereby releasing a temporary p53 inhibitor.

The invention also provides a compound of Formula VI:

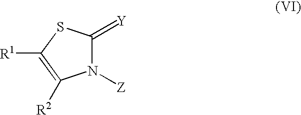

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Y and Z taken together complete a 5-member imidazole ring of Formula VII or Formula VIII,

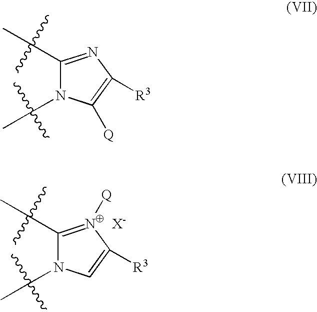

wherein X⁻ is selected from the group consisting of a chloride, a bromide, a fluoride, an iodide, an acetate, a formate, a phosphate, a sulfate, and other pharmaceutically acceptable anions, and Q is an organic moiety that contains a nucleophilic or electrophilic reacting group and is cleavable under physiological conditions.

The invention provides a compound of Formula V:

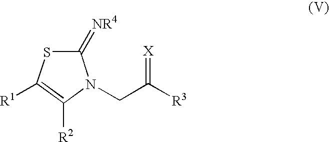

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. X is A-J, a carbonyl, or a protected carbonyl. R⁴ is hydrogen or an C₁-C₆ acyl group when X is A-J or R⁴ is A-J when X is a carbonyl or protected carbonyl. A is an organic moiety that is cleavable under physiological conditions, and J is a bone targeting agent.

In addition, the invention provides a compound of Formula VI:

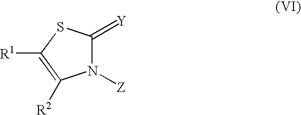

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Y and Z taken together complete a 5-member imidazole ring of Formula VII or Formula VIII,

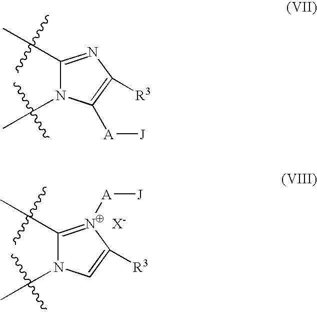

wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyls, C₁-C₆ alkoxy, hydroxy, fluoro, chloro, bromo, nitro, amino, C₁-C₆ alkylamino, and/or C₄-C₁₄ aromatic or heteroaromatic moieties, and X⁻ is selected from the group consisting of a chloride, a bromide, a fluoride, an iodide, an acetate, a formate, a phosphate, a sulfate, and other pharmaceutically acceptable anions. A is an organic moiety that is cleavable under physiological conditions, and J is a bone targeting agent.

The invention further provides a compound of Formula IX:

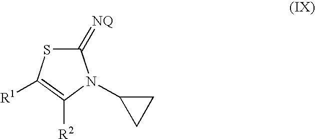

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Q is an organic moiety that contains a nucleophilic or electrophilic reacting group and is cleavable under physiological conditions.

### BRIEF DESCRIPTION OF THE DRAWINGS

FIG. 1 is an illustration of the chemical structure of pifithrin-α.

FIG. 2 is an illustration of the chemical structure of pifithrin-β.

FIG. 3 is an illustration of the chemical structure of EDTMP.

FIG. 4 is an illustration of the chemical structure of DOTMP.

FIG. 5 is an illustration of the chemical structure of ABDTMP.

FIG. 6 is an illustration of the chemical structure of BAD.

FIG. 7 is an illustration of the chemical structure of MTX-BP.

FIG. 8 is an illustration of the chemical structure of CF-BP.

FIG. 9 is an illustration of the chemical structure of ACL-3.

FIG. 10 is an illustration of a chemical reaction for producing pifithrin-α.

FIG. 11 is an illustration of possible reversible modifications of pifithrin-α.

FIG. 12 is an illustration of imine protection or deprotection reactions.

FIG. 13 is an illustration of potential imine acylation reactions of pifithrin-α.

FIG. 14 is an illustration of potential reverse Mannich bases of pifithrin-β.

FIG. 15 is an illustration of a chemical reaction for derivatization of pifithrin-α with ACL-3 and acidic cleavage thereof.

FIG. 16 is an illustration of the chemical structure of cis-aconitic anhydride.

FIG. 17 is an illustration of the chemical structure of a linker-bone targeting agent conjugate.

FIG. 18 is an illustration of a chemical reaction for forming a bis-ortho ester linker.

FIG. 19 is an illustration of a chemical reaction for generating succinamic ester linkers.

FIG. 20 is an illustration of chemical structures of bone targeting agents.

FIG. 21 is an illustration of chemical structures of bone-targeting reversible prodrugs of pifithrin-α.

FIG. 22 is an illustration of a chemical reaction for preparing an intermediate in the synthesis of a pifithrin-β pro-drug.

FIG. 23 is an illustration of a chemical reaction for preparing reversible acyl methylenes and conversion thereof to pifithrin-β.

FIG. 24 is an illustration of a chemical reaction for preparing an analog of DOTMP.

FIG. 25 is a bar graph illustrating the percent of compound present following incubation with 0 mg/ml, 1 mg/ml, or 10 mg/ml hydroxyapatite as described in Example 9.

FIG. 26 is an illustration of a chemical reaction for preparing a bone targeted compound of the invention.

FIG. 27 is a bar graph illustrating the fold increase of p53-mediated transcription in human brain endothelial cells in the presence and absence of pifithrin-β.

FIG. 28 is an illustration of a coupling chemical reaction.

FIG. 29 is an illustration of an alkylation reaction.

FIG. 30 is an illustration of a chemical reaction for preparing a bone-targeting group.

FIG. 31 is an illustration of chemical structures of bone-targeting groups and cell protection factor-bone targeting group conjugates.

FIG. 32 is an illustration of chemical structures of bone-targeting groups and cell protection factor-bone targeting group conjugates.

FIG. 33 is an illustration of chemical structures of bone-targeting groups and cell protection factor-bone targeting group conjugates.

FIG. 34 is an illustration of chemical structures of bone-targeting groups and cell protection factor-bone targeting group conjugates.

FIG. 35 is an illustration of chemical structures of bone-targeting groups and cell protection factor-bone targeting group conjugates.

### DETAILED DESCRIPTION OF THE INVENTION

Many medical treatments aimed at controlling abnormal cell proliferation, e.g., cancer treatments, interfere with natural cellular processes to provoke programmed cell death. In that normal cell processes are affected, both abnormal and normal cells are susceptible to the effects of such therapeutics. As such, the choice of therapeutic, dosage, and time of treatment is often limited by adverse effects on normal tissues. This is particularly relevant in cancer treatments which can involve high doses of chemotherapy and radiation therapy, which causes intense sickness in patients. Blockage of even a percentage of the toxic side effects caused by destructive medical treatments can allow practitioners to pursue more aggressive treatment regimens than otherwise tolerated by a subject or allow patients to be treated that otherwise are deemed to be untreatable. The invention provides a method of inhibiting cell death in a mammal, such as death of normal cells in response to drugs, e.g., chemotherapy and radiation. The method comprises administering to a mammal an effective amount of a composition comprising a cell protection factor covalently linked to a bone targeting agent via a linker that is cleaved under physiological conditions. Desirably, when linked to the bone targeting agent, the cell protection factor is inactive with respect to blocking apoptosis. Under physiological conditions, the cell protection factor is released from the bone targeting agent in active form to inhibit cell death. By targeting to bone, the inventive method allows delivery of a cell protection factor to bone marrow while minimizing the risk of uncontrolled cell proliferation which may result from widespread exposure of the body to a cell protection factor. Bone marrow is the source of a variety of cell types, including hematopoietic cells (e.g., reticulocytes and erythrocytes) and immune cells, such as leukocytes, which include, for instance, polymorphonuclear granulocytes (e.g., neutrophils, eosinophils, and basophils), monocytes, and lymphocytes (e.g., B cells, T cells, and natural killer (NK) cells). Protection of bone marrow can greatly enhance the quality of life of patients undergoing destructive medical treatment by reducing or eliminating damage to normal tissue, in particular bone marrow, attributable to, for example, treatment for cancer, radiation exposure, and the like.

By “cell protection factor” is meant any factor (e.g., small molecule or peptide) which inhibits apoptosis in a cell. The apoptosis can be induced by environmental or chemical insults such as, for example, exposure to pro-apoptotic agents, physical damage of the cellular machinery, radiation exposure, viral or bacterial infection, or chemotherapy. Preferably, the cell protection factor inhibits the activity of a tumor suppressor gene. Tumor suppressor genes include, for example, RB1, p53, INK4a, e.g., p16 and p19, APC, BRCA1, BRCA2, WT1, NF1, NF2, VHL, MEN1, PTCH, PTEN/MMAC1, DPC4, E-CAD, LKB1/STK1, SNF5/INI1, EXT1, Waf, p27, Myc, MDM-2, EXT2, TSC1, TSC2, MSH2, MLH1, PMS2, PMS2, and MSH6. It has also been proposed that TGF-β type II R, BAX, FHIT, α-CAT, DCC, MADR2/SMAD2, CDX2, MKK4, PP2R1B, and MCC also have tumor suppressor activity (Holland et al., eds., Cancer Medicine, 5^{th} ed., B. C. Decker Inc., Hamilton, Ontario (2000)). More preferably, the cell protection factor inhibits the activity of p53. To guard against uncontrolled proliferation of cells, the cell protection factor desirably is a temporary inhibitor of a tumor suppressor gene, e.g., p53 and/or its encoded protein, p53, meaning that the inhibitory effect of the cell protection factor on p53 is not permanent. In other words, the effect of the cell protection factor on p53 is reversible. For example, the cell protection factor can inhibit p53 activity in at least one cell sensitive to p53 inhibition for at least about 30 minutes (e.g., at least about 1 hour, at least about 3 hours, at least about 6 hours, or at least about 12 hours) and not longer than about 14 days (e.g., not longer than about 12 days, not longer than about 10 days, not longer than about 7 days, or not longer than about 5 days) following contact with a target cell. More preferably, the cell protection factor (e.g., a temporary p53 inhibitor) inhibits tumor suppressor gene product (e.g., p53) activity for at least about 18 hours (e.g., at least about 24 hours, at least about 36 hours, at least about 48 hours, or at least about 60 hours) and not longer than about 6 days (e.g., not longer than about 5 days or not longer than about 4 days) following contact with a target cell. Even more preferably, the cell protection factor interrupts, for example, p53 activity for about 48-96 hours, e.g., 72 hours.

The nuclear transcription factor p53 serves as an important part of the cellular emergency response mechanism ultimately leading to apoptosis, such as apoptosis in response to chemotherapy, radiation, and other forms environmental stress. Inhibition of p53 in the bone marrow in the context of the invention is valuable for protecting cells of the bone marrow during chemotherapy and radiotherapy treatment of virtually any tumor (except those residing in the bone and marrow) including those possessing competent p53 regulatory mechanisms. Approximately 50% of cancers are p53-deficient. The method and composition of the invention has particular utility as an adjunct therapy for treatment of p53-deficient tumors. Because p53-deficient tumors are not always easily discernable, the invention further allows for the broad use of targeted p53 inhibition such that the inhibition of p53 is limited to the bone and bone marrow compartments. In other words, composition of the invention can be administered to a mammal comprising at least one tumor. The tumor can be a p53-deficient (p53−) or p53-positive (p53+) tumor. The bone-seeking “prodrug” of the invention can be thought of as a conjugate or three piece construct, p531-CL-BSA, wherein p531 is an inhibitor of p53, CL is a covalent linkage capable of in vivo cleavage to generate the free, active p53 inhibitor entity, and BSA is the bone seeking portion of the construct. By administering the active agent in the inventive composition in prodrug form, i.e., an inactive form by virtue of the linkage to the bone targeting agent, the risk of inadvertently rendering p53+tumors resistant to chemotherapy is minimized. Since p53 is important in controlling cell proliferation and apoptosis under conditions of chemotherapy or radiation or other stress stimuli, the bone targeting approach of the invention impacts on preservation of bone marrow function under conditions of cellular stress. Accordingly, such inhibitors provide protection of bone marrow function and allow for higher doses of chemotherapy to be administered more safely to the patient.

The method and compounds of the invention find utility in a variety of contexts in addition to inhibition, reduction, or prevention of cell death associated with medical exposure to cell destructive agents. Non-medical exposure to cell killing agents can occur in research laboratories, power plants, radiology offices, military situations, and in the course of public service (e.g., police, firefighters, and National Guard). For example, the method and compounds of the invention can be used by military personnel to guard against biological damage (e.g., bone marrow destruction) caused by exposure to, for example, radiation or biological weapons. In this respect, it is envisioned that the inventive method and compounds can be provided to military, police, or firefighter personnel prior to encountering contaminated areas. In addition, the inventive method and compounds can be provided to a population at risk of terrorist attack to curb the biological damage, including suppression or ablation of the hematopoietic and/or immune system, caused by biological weapons.

The protective activity of the cell protection factor need not be used in response to exogenous, environmental stimuli. Many processes in the body can result in cell damage, which can be inhibited by administration of the compounds of the invention. For example, ischemia and ischemia/reperfusion injury can be minimized by a cell protection factor of the invention. Ischemia is often caused by an interruption of the supply of oxygenated blood, such as that caused by a vascular occlusion. Vascular occlusions can be caused by arteriosclerosis, trauma, surgical procedures, disease, and/or other indications. Many methods of identifying a tissue at risk of suffering ischemic damage are available. Such methods are well known to physicians who treat such conditions and include, for example, a variety of imaging techniques (e.g., radiotracer methodologies such as 99 mTc-sestamibi scanning, x-ray, and MRI scanning) and physiological tests. In inhibiting cell death associated with, for example, peripheral vascular disease, the compounds of the invention can be used to target the cell protection factor to bone tissue adjacent to affected musculature suffering from or at risk of suffering from ischemia/reperfusion injury. In treating, for example, myocardial ischemia, the compounds of the invention can bind to arterial calcium deposits for release of the cell protection factor in the vicinity of the myocardium.

In addition to use as a protectant, the methods and compounds of the invention also may stimulate bone marrow recovery under certain physiologic conditions of bone marrow failure. The compounds of the invention also can enable bone marrow transplantation for non-bone marrow infiltrative diseases (brain tumors, breast cancer) without the requirement for myelosuppression.

The cell protection factor of the invention can block the functioning of a tumor suppressor gene by one or more suitable means, such as by inhibiting production of the encoded protein (e.g., by inhibiting transcription or translation of the encoded tumor suppressor protein), by interfering with the active site of the tumor suppressor protein, reducing the nuclear accumulation of the tumor suppressor protein, or by blocking the cascade of intracellular events mediated by the tumor suppressor protein. The desired biological effect of the cell protection factor in vivo following release from the bone targeting agent is inhibition of cell death (e.g., apoptosis), preferably bone marrow cell death in response to environmental stress or chemical insult. Thus, the activity of a cell protection factor can be evaluated by detecting a disruption in tumor suppressor protein-mediated signaling (e.g., p53-mediated cell signaling) or by evaluating cell death in the presence and absence of the cell protection factor. Accordingly, cell protection factors can be screened using assays that detect cell death (e.g., apoptosis). For example, apoptosis can be evaluated in a cell or tissue sample using a variety of commercially-available kits, such as the ApoAlert™ family of products (BD Biosciences, Palo Alto, Calif.) and DNA laddering and in situ labeling kits available from, for example, R&D Systems, Minneapolis, Minn., as well as methods commonly used in the art, such as the TUNEL (Terminal deoxynucleotidyl transferase mediated dUTP nick end labeling) assay and others described in, e.g., Zhu and Chun, eds., Apoptosis Detection and Assay Methods, Biotechniques Press, Westborough, Mass. (1998).

In the context of the invention, it will be appreciated that complete prevention of cell death is not required to achieve a biological or therapeutic effect. Accordingly, the inventive method does not require a complete prevention of cell death. Preferably, the cell protection factor mediates at least a 5% reduction or inhibition of cell death (e.g., at least a 10%, at least a 15%, or at least a 20% reduction or inhibition of cell death) in a sample compared to a biologically-matched sample which is not exposed to the cell protection factor. More preferably, the cell protection factor mediates at least a 25% reduction or inhibition of cell death (e.g., at least a 30%, at least a 35%, at least a 40%, or at least a 45% reduction or inhibition of cell death) in a sample compared to a biologically-matched sample which is not exposed to the cell protection factor. Even more preferably, the cell protection factor mediates at least a 50% reduction or inhibition of cell death (e.g., at least a 55%, at least a 60%, at least a 65%, or at least a 70% reduction or inhibition of cell death) in a sample compared to a biologically-matched sample which is not exposed to the cell protection factor. Even more preferably, the cell protection factor mediates at least a 75%, at least an 80%, at least an 85%, at least a 90%, at least a 95%, or 100% reduction or inhibition of cell death in a sample compared to a biologically-matched sample which is not exposed to the cell protection factor. Measurement timepoints in assays for calculating the level of cell protection can be determined by the practitioner based on a variety of factors including adjunct treatment regimens, route of administration of the cell protection factor-bone targeting agent conjugate, cleavage kinetics of the linker joining the cell protection factor and the bone targeting agent, desired duration of inhibition of the tumor suppressor gene (and cell death), and desired time to onset of biological effect. Cell protection factors can be screened in vitro by, for example, contacting target cells (e.g., bone marrow cells) with a cell protection factor in cell culture. When creating a multitude of cell protection factors using, for example, combinatorial chemistry, high-throughput screening techniques allow rapid in vitro screening. Alternately, a cell protection factor can be administered in situ or in vivo, e.g., directly to a tissue graft or target tissue, and samples can be harvested and screened ex vivo. A cell protection factor can be selected by the practitioner according to a desired level of cell protection at a desired time following contact of target cells.

In one embodiment, the cell protection factor is a compound of Formula I:

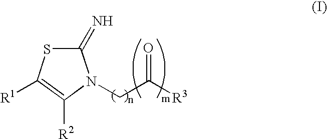

wherein m is 0 or 1, n is an integer from 1 to 4, R¹ and R² are taken together to form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties, and R³ is selected from the group consisting of a C₁-C₆ alkyl group, a C₁-C₆ alkoxy group, and a phenyl group. The alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and optionally forms a C₃-C₆ cycloalkyl when R³ is connected to the alpha carbon to the thiazole ring.

By “alkylthio” is meant an organic radical derived from an open, straight or branched hydrocarbon chain wherein the terminus of the organic radical terminates in a —SH group (thiol group).

By “aliphatic” is meant an organic radical derived from an open, straight, or branched hydrocarbon chain. Examples of aliphatic moieties include, for example, alkanes, alkenes, and alkynes (e.g., C₁-C₆ alkyl radicals, straight or branched chains).

By “aromatic” is meant a monocyclic or polycyclic set of unsaturated carbons, e.g., phenyl. Similarly, “heteroaromatic” is a monocyclic or polycyclic set of carbons wherein one or more carbons is replaced with a nitrogen, oxygen, or sulfur atom. Examples include, but are not limited to, furyl, pyridyl, pyramidyl, quinolyl, thienyl, and thiazyl groups. It is understood that the term aromatic applies to cyclic substituents that are planar and comprise 4n+27π electrons, according to Hückel's Rule.

By “alkyl” is meant a straight or branched chain of saturated or unsaturated carbons. Examples include, but are not limited to, methyl, ethyl, ethenyl, n-propyl, isopropyl, cis-propenyl, trans-propenyl, propynyl, n-butyl, isobutyl, sec-butyl, tertiary-butyl, 2-cis-butenyl, 2-trans-butenyl, n-pentyl, isopentyl, n-hexyl and the like. The term “alkyl” is also meant to include cycloalkyl and cycloalkenyl moieties (e.g., “C₁-C₆ alkyl” encompasses cycloalkyl and cycloalkenyl moieties of 3 to 6 carbons).

By “alkoxy” is meant an —OR group, wherein R is alkyl or aryl.

By “amino” is meant an —NH₂ group. By “alkylamino” is meant an —NH₂ substituted with one or two C₁-C₆ alkyl or aryl groups. Examples include, but are not limited to, amino, methylamino, dimethylamino, diethylamino, methylethylamino, or phenylamino.

By “acyl” is meant a carbonyl-R group, i.e., —C(═O)—R, wherein the carbonyl is bound to an alkyl group and a heteroatom.

In one embodiment, the cell protection factor is a compound of Formula I, wherein m is 0, n is 2, and R³ is a one-carbon alkyl such that the three-carbon chain forms a cyclopropyl group. In other words, the cell protection factor is a compound of Formula X:

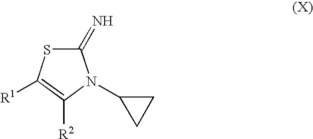

where R¹ and R² are taken together to form an aliphatic or aromatic carbocyclic 5- to 8-membered ring optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties.

In one embodiment, the cell protection factor is a compound of Formula I or Formula X, wherein R¹ and R² are taken together to form a 5- or 6-membered aliphatic carbocyclic ring. The 5- or 6-membered aliphatic carbocyclic ring optionally is substituted with one or more C₁-C₆ alkyl groups.

In a preferred embodiment, the cell protection factor is a compound of Formula II:

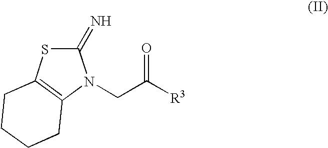

wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic groups. Even more preferably, the cell protection factor is a compound of Formula III:

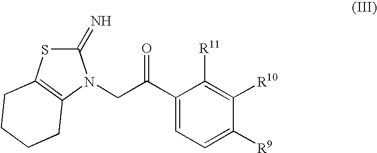

wherein R⁹, R¹⁰, and R¹¹ are each independently a hydro, hydroxyl, methyl, fluoro, chloro, bromo, nitro, amino, methoxy, or phenyl. Examples of the substitution around the aromatic ring include, but are not limited to, 2-, 3-, and 4-methyl, 2-, 3-, and 4-methoxy, 2-, 3-, and 4-nitro, amino, 2,4-dimethyl, 3,4-dimethyl, 2-methoxy-3-methyl, 2-methoxy-4-methyl, 3-methoxy-4-methyl, 2-methyl-3-methoxy, 2-methyl-4-methoxy, 3-methyl-4-methoxy, 2-, 3-, and 4-chloro, 2-, 3-, and 4-fluoro, 2-, 3-, and 4-hydroxy. Desirably, the cell protection factor is 2-[2-imino-4,5,6,7-tetrahydro-1,3-benzothiazol-3(2H)-yl]-1-(4-methylphenyl)-1-ethanone (i.e., pifithrin-α) or 2-[2-imino-4,5,6,7-tetrahydro-1,3-benzothiazol-3(2H)-yl]-1-(biphenyl)-1-ethanone.

A cell protection factor in the context of the invention also can be a compound of Formula IV:

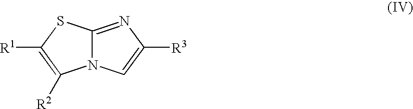

wherein R¹ and R² are taken together to form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Preferably, the cell protection factor is 2-p-Tolyl-5,6,7,8-tetrahydro-benzo[d]imidazo[2,1-b]thiazole (i.e., pifithrin-β), which is a cyclized derivative of pifithrin-α.

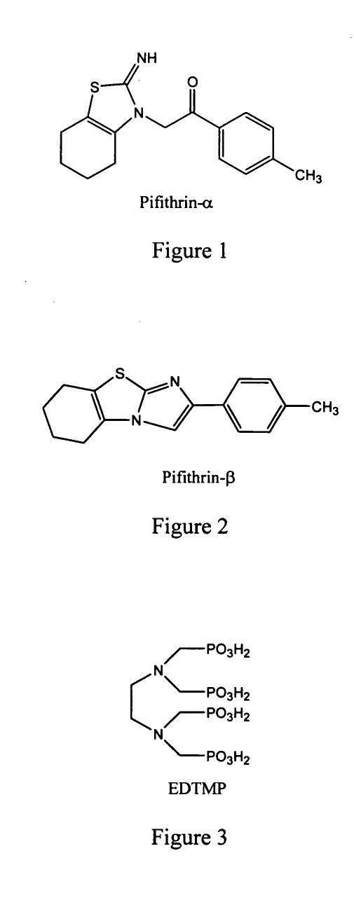

Pifithrin-α (PFT-α; the chemical structure of which is set forth in FIG. 1) was identified in the course of pioneering work based on the hypothesis that blockage of the p53 protein on a temporary basis in an animal with p53-deficient tumors could prevent p53-initiated cell death in normal tissues and, hence, prevent many of the side effects associated with chemotherapy and/or radiation treatments (Komarov et al., Science, 285, 1733 (1999)). A 10 micromolar concentration of PFT-α inhibited apoptosis in C8 cells (mouse embryo fibroblasts transformed with E1a+ras) induced by doxorubicin, etoposide, paclitaxel, cytosine arabinoside, UV light, and gamma radiation. To be effective, it was demonstrated that PFT-α must be present during or immediately (less than 3 hours) after exposure to, for example, UV radiation to provide a cell-protective effect. Pretreatment of cells with PFT-α and removal of the compound before the stress-inducing event provided no significant protection. PFT-α was also tested in two different strains of mice, with PFT-α administered as a single intraperitoneal injection (2.2 mg/kg of body weight). Remarkably, PFT-α completely rescued both types of mice from 60% killing doses of gamma radiation (8 Gy for C57BL strain and 6 Gy for Balb/c strain). Additionally, the treated animals experienced less weight-loss than controls. Importantly, in p53-null mice controls treated with radiation, PFT-α injections had no protective effect. No tumors or pathological lesions were found in the PFT-α treated, gamma-irradiated survivors after 7 months post-irradiation. PFT-α and PFT-β is further described in U.S. Pat. No. 6,593,353; U.S. Patent Application Publication Nos. 2002/0006941, 2003/0073611, and 2003/0144331; and International Patent Application WO 99/41985.

Pifithrin-α, administered systemically, can convert to the cyclized derivative, pifithrin-β (PFT-β; the chemical structure of which is set forth in FIG. 2). The conversion (i.e., dehydrative cyclization) of PFT-α to PFT-β has been described and, in terms of preparatory methodology, can be effected in high yields in heated alcohol solvents (see, for example, International Patent Application WO 00/44364). PFT-β is less toxic than PFT-α, and is similar in p53 inhibitory activity to PFT-α. The decreased toxicity of PFT-β relative to PFT-α can result from loss of a potentially bio-reactive ketone group. Thus, masking the ketone of PFT-α could serve to diminish toxicity, as well as serve as a linkage point to bone-seeking groups.

Heterocyclic compounds similar to PFT-α can interact with alkaline phosphatase (see, for example, U.S. Pat. No. 5,516,647), aid in glutamate transmission in epilepsy (see, for example, International Patent Application WO 93/01194), and influence multidrug resistance via P-glycoproteins (see, for example, Tasaka et al., J Heterocyclic Chem., 34, 1763 (1997)), which are believe to adversely affect cell functioning. The invention limits such side-effects associated with systemic administration of PFT-α (as well as other tumor suppressor gene inhibitors) by covalently attaching the molecule to a bone targeting agent. Furthermore, the invention allows for treatment of p53-competent tumors so long as the tumors do not reside in the bone and bone marrow.

The cell protection factor of the inventive method is covalently linked to a bone targeting agent via a linker that is cleaved under physiological conditions. By “bone targeting agent” is meant a ligand (e.g., a chemical moiety or peptide) that reversibly binds to bone tissue and is not toxic to a mammal, especially a human. For instance, the bone targeting agent can be a ligand that binds hydroxyapatite, a major component of bone and dental structures. The compound of the invention can be targeted to calcium deposits in regions of the body other than bone, such as calcium deposits in the arteries, heart, kidney, or gall bladder. However, the bone targeting agent ideally selectively binds to bone tissue. In other words, the bone targeting agent preferably binds to bone tissue with at least 2-fold greater affinity (e.g., at least 3-fold, at least 5-fold, at least 10-fold, or at least 25-fold greater affinity) than the bone targeting agent binds to non-bone tissue. The bone targeting agent reversibly binds to bone tissue, meaning that the bone targeting agent is eventually released from bone and expelled from the body. The bone targeting agent remains bound to bone tissue for a sufficient period of time to allow cleavage of the linker and release of a desired dose of cell protection factor to target cells (e.g., bone marrow cells). The bone targeting agent can remain bound to bone for about 10 minutes (e.g., about 20 minutes, about 30 minutes, about 1 hour, or about 3 hours) to about 6 months (e.g., about 90 days, about 120 days, or about 150 days) after cleavage of the linker, after which the bone targeting agent is expelled from the body. The bone targeting agent can remain bound to bone for about 6 hours (e.g., about 12 hours, about 24 hours, about 48 hours, about 3 days, about 7 days, or about 14 days) to about 60 days (e.g., about 30 days or about 45 days) post-cleavage of the linker. For example, in a conjugate wherein a cell protection factor is covalently attached to a bone targeting agent via a linker for which 50% is cleaved in vivo after approximately 72 hours post-administration, the bone targeting agent ideally remains bound to bone for at least 72 hours post-administration. More preferably, the bone targeting agent remains bound to bone for a sufficient amount of time to allow at least 75% (e.g., at least 85%, at least 90%, at least 95% or 100%) of the linker to cleave and release the cell protection factor. To ensure maximal delivery of the cell protection factor, the bone targeting agent remains bound approximately 1-5 hours longer than required for cleavage of 100% the linker. Thus, a bone targeting agent for use in the invention can be selected based on binding kinetics to bone tissue. Candidate bone targeting agents can be screened in vitro by determining affinity to bone tissue (e.g., hydroxyapatite) in, for example, a multi-well format. Candidate bone targeting agents also can be screened in vivo by assessing the rate and timing of excretion of candidate bone targeting agents from the body. In this respect, the bone targeting agent preferably is expelled from the body via the kidneys.

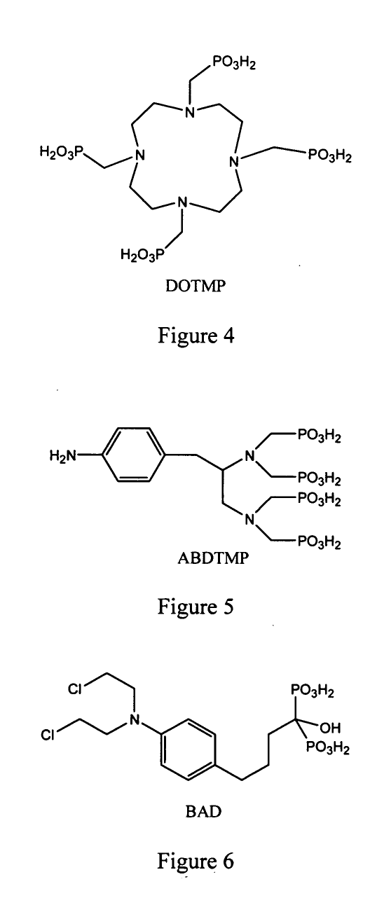

The bone targeting agent desirably is selected from the group consisting of a bisphosphonate, a hydroxybisphosphonate, a phosphonate, a phosphate, an aminomethylenephosphonic acid, and an acidic peptide. The bone targeting agents of the invention are envisioned to carry one or more of these groups. For example, the bone targeting agent can be a phosphonate, meaning that the bone targeting agent may comprise one phosphonate, two phosphonates, or three or more phosphonates. One suitable bone targeting agent for use in the invention is EDTMP (the chemical structure of which is set forth in FIG. 3), currently FDA approved (Quadramet™) as the radioactive ¹⁵³Sm complex for delivering a selective radiation dose to bone metastases for pain palliation. EDTMP is a phosphonate that contains four phosphonic acid groups, and is therefore a tetraphosphonate. Another suitable bone targeting agent (or bone-targeting system) is DOTMP (the chemical structure of which is set forth in FIG. 4) in Phase III clinical trials (termed STR, skeletal targeted radiation) sponsored by NeoRx as the radioactive ¹⁶⁶Ho complex designed to deliver large doses of radiation selectively to the bone marrow for the treatment of multiple myeloma. Polyphosphonic acids and aminomethylenephosphonic acids have a high affinity for bone in vivo due to their binding of the exposed calcium ions in hydroxyapatite (calcium phosphate), and also are suitable for use in the context of the invention.

The terms “phosphonate, phosphate, and aminomethylenephosphonate” are meant to encompass the phosphonic acids, the phosphoric acids, and aminomethylenephosphonic acids, respectively, as well as any salts, hydrolyzable esters, and prodrugs of the phosphorous-based acids thereof. At the biological pH of 7.4 in the blood, or the more acidic pH around the bone, a certain portion of the phosphate or phosphonate of the bone targeting agent may be deprotonated and replaced with a counterion. Furthermore, the exchange of proton for calcium is an inherent event for the binding of the bone targeting agent to the hydroxyapatite in the invention. However preparation and administration of the composition containing the bone targeting agent may or may not require complete protonation of the phosphorous acids therein. Therefore, the phosphonic acid, phosphoric acid, and aminomethylenephosphonic acid are drawn and utilized interchangeably with phosphate, phosphonate, and aminomethylenephosphonate. While not particularly preferred, biologically hydrolyzable esters of the phosphorus-based acids may also be utilized in the method of the invention. Similarly, prodrugs of the phosphorous-based acids may also be utilized in vivo to mask the acidity of the composition during, for example, formulation and administration.

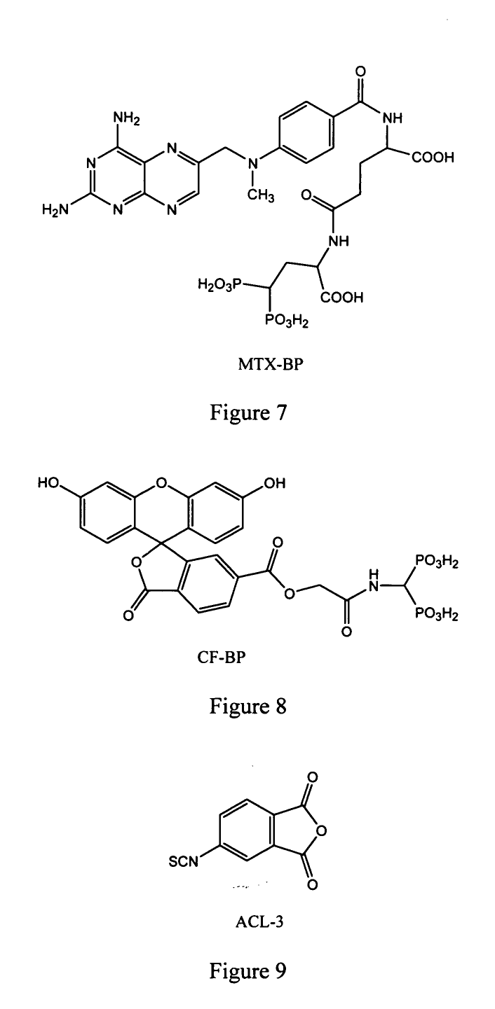

Preferably, the bone targeting agent is a polyphosphonic acid. Polyphosphonic acid has been demonstrated to successfully target biologically-active molecules to bone tissue. For example, conjugation (via isothiocyanato chemistry) of polyaminophosphonic acids, such as ABEDTMP (the chemical structure of which is set forth in FIG. 5), to growth factors (to stimulate bone formation) successfully resulted in the targeting of the growth factors to the bones of rats (see, for example, International Patent Application WO 94/00145). The utility of bone-seeking agents extends beyond delivery of proteins to bone and includes, for instance, small therapeutic molecules. A conjugate comprising a bone-seeking bisphosphonate and an alkylating agent, such as BAD (the chemical structure of which is set forth in FIG. 6), has been generated (see, for example, Wingen et al., J. Cancer Res. Clin. Oncol., 111, 209 (1986)). In this molecule, the alkylating agent is not specific in its interaction with its target (DNA), and, thus, there is no requirement for cleavage between the bisphosphonate (i.e., bone-seeking agent) and the alkylating moiety. The bisphosphonate-alkylating agent demonstrated efficacy in a rat osteosarcoma model using BAD. A series of studies was performed on the antifolate antineoplastic agent, methotrexate, covalently attached to an number of bisphosphonates, as exemplified by the structure designated MTX-BP12, the chemical structure of which is set forth in FIG. 7 (see, for example, Sturtz et al., Eur. J. Med. Chem., 27, 825 (1992); Sturtz et al., Eur. J. Med. Chem., 28, 899 (1993); and Hosain et al., J Nucl. Med., 37, 105 (1996)). Using Tc-99m labeled MTX-BP, it was determined that around 15% of the injected dose was localized in the skeleton after 4 hours with about 61% of the dose being excreted (Hosain, supra). MTX-BP further demonstrated five times greater anticancer activity compared with methotrexate alone in animal models of transplanted osteosarcoma (Sturtz 1992, supra). Similar work has been described using the conjugate CF-BP, the chemical structure of which is set forth in FIG. 8 (Fujisaki et al., Journal of Drug Targeting, 4, 117 (1994)). In this molecule, the CF (carboxyfluorescein) group is a fluorescent marker to quantitate pharmacokinetics and biodistribution, and is connected to the bone targeting agent through an ester bond which is susceptible to hydrolysis in vivo. Studies in rats injected intravenously indicated that CF-BP localized in the bone and served as a slow release mechanism for CF generated via general hydrolysis of the ester linkage (Fujisaki, supra).

In another embodiment, the bone-seeking agent can be a peptide, such as (Asp)₆ and (Glu)₆. The acid-rich peptide sequence of the glycoprotein osteonectin, which is found in abundance in bone and dentin, has a strong affinity to hydroxyapatite (Fujisawa et al., Biochimica et Biophysica Acta, 53, 1292 (1996)). Thus, peptide ligands comprising acidic amino acids are ideal candidates for bone targeting agents. Indeed, (Glu)₁₀, when attached to biotin, successfully recruited labeled strepavidin to hydroxyapatite (described further in Chu and Orgel, Bioconjugate Chem., 8, 103 (1997), and International Patent Application WO 98/35703). In addition, the biological half-life of the fluorescein isothiocyanate conjugated to (Asp)₆ was 14 days in the femur (Kasugal et al., Journal of Bone and Mineral Research, 15(5), 936 (2000)), which is an acceptable half-life for the bone targeting agent of the invention. Likewise, delivery of estradiol-(Asp)₆ conjugates to bone has been demonstrated in ovariectomized animals with concomitant inhibition of osteoporectic-type bone loss (Kasugai et. al., Journal of Bone and Mineral Research (Suppl 1), 14, S534 (1999)). It is believed that the (Asp)₆ tether to bone is metabolized during the bone resorption process mediated by osteoclasts. Therefore, the acidic peptide ligand provides not only a means of recruiting compounds to bone, but also provides a mechanism of slowly releasing compounds to bone cells and surrounding tissue.

Other examples of bone targeting agents include, but are not limited to, amino- and hydroxy-alkyl phosphonic and diphosphonic acids; hydroxybisphosphonic acids including alendronate, pamidronate, 4-aminobutylphosphonic acid, 1-hydroxyethane-1,1-diphosphonic acid, and aminomethylenebisphosphonic acid; phosphates such as phytic acid; and aminomethylenephosphonic acids such as N,N-bis(methylphosphono)-4-amino-benzoic acid and nitrilotri(methylphosphonic acid). It is envisioned that these bone targeting agents can be attached through one of the heteroatoms or by chemical modification that installs an additional attachment point.

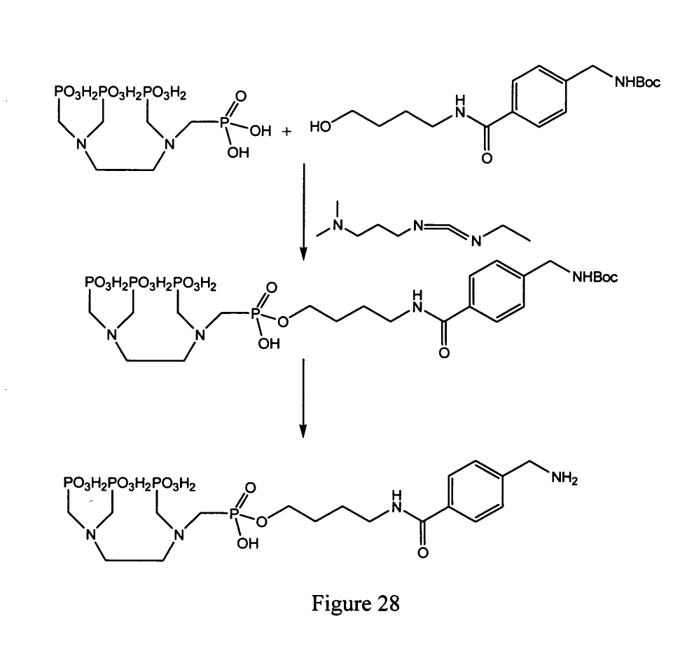

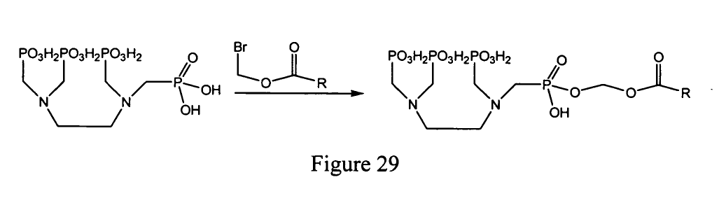

DOTMP and EDTMP (ethylene diamine-N,N,N′,N′-tetrakis(methylenephosphonic acid) are bone seeking agents which must be converted into a derivative capable of attaching to the cell protection factor. Derivatization can be performed by a variety of chemical processes, such as the coupling chemistry shown in FIG. 28 and alkylation chemistry shown in FIG. 29, where the R group can have, for example, a phenylcarboxylic acid group, to react with the cell protection factor. The coupling chemistry illustrated in FIG. 28 is further described in Vieira de Almedia et al., Tetrahedron, 55, 12997-13010 (1999). Alkylation chemistry involving DOTMP has been further described in Chavez et al., Biomedical Imaging: Reporters, Dyes, & Instrumentation, Contag & Sevick-Muracia, Eds., Proc. SPIE, Vol. 3600, 99-106 (July, 1999). Alkylation chemistry for other phosphonic acids is further described in, for example, U.S. Pat. No. 5,177,064, U.S. Pat. No. 5,955,453, de Lombaert et al., J Med Chem., 37, 498-511 (1994), and Iyer et al., Tetrahedron Letters, 30(51), 7141-7144 (1989). EDTMP can be connected to the linker by one of the phosphorous oxygens to create a phosphonate linkage. Alternatively, EDTMP can be chemically modified to generate ABDTMP by installation of an aniline group (as further described in, for example, FIG. 5 of International Patent Application WO 94/00145). The aniline amine is then available to form, for example, an amide bond with a linker and attach to the cell protection factor.

The bone delivery technology described herein can include the delivery of compounds to bone (and bone marrow) that are capable of converting mutant inactive p53 into active p53, thereby rendering such cells sensitive to chemo- and radiation-treatment. One such low-molecular weight molecule has recently been described (Foster et al., Science, 286, 2507 (1999)) to convert mutant inactive p53 into active p53. Alternatively, a combination of the bone targeted-p53 inhibitor described herein with a tumor localizing, small molecule p53 activator is a potent way to treat p53 mutant tumors and spare the marrow from toxicity.

The cell protection factor of the inventive method is covalently attached to the bone targeting agent via a linker that is cleavable under physiological conditions. The cell protection factor ideally is inactive when conjugated to the bone targeting agent through the cleavable linker. Upon administration to a mammal, the cell protection factor-bone targeting agent conjugate attaches to bone tissue (or another calcium-containing structure), the linkage between the moieties is cleaved, and the cell protection factor (e.g., temporary p53 inhibitor) is released in active form to inhibit cell death in the surrounding area. The conjugate comprising the cell protection factor covalently linked to a bone targeting agent can, therefore, be considered a “prodrug,” which is activated upon cleavage of the linker and release of the active cell protection factor.

In one embodiment, the linker comprises an organic moiety comprising a nucleophilic or electrophilic reacting group which allows covalent attachment to the bone targeting agent. Preferably, the linker is an enol ether, ketal, imine, oxime, hydrazone, semicarbazone, acylimide, or methylene radical. The selection of a particular linker will depend on the target environment and the desired release kinetics of the cell protection factor from the bone targeting agent. Desirably, the linker is an acid-cleavable linker, a hydrolytically cleavable linker, or enzymatically-cleavable linker. By “acid-cleavable” is meant that the linkage between the cell protection factor and bone targeting agent is cleaved below pH 7. For example, an acid cleavable linker, such as ACL-3 (the chemical structure of which is set forth in FIG. 9), can link a bone targeting agent and a cell protection factor. In linking a protein moiety (e.g., a cell protection factor) to a bone targeting agent, the anhydride group of an acid-cleavable linker, such as ACL-3, reacts first with the free amino group of the protein (e.g., a cell protection factor (such as the N—H of alpha-pifithrin)). In a subsequent step, the isothiocyanato is reacted with the amino groups of the bone-seeking moiety at higher pH to create a stable thiourea linkage. Under acidic conditions, the protein-ACL-3 amide linkage is readily cleaved, freeing the native amino group of the protein. When PFT-α is attached to the bone targeting group, the ACL-3 amid linkage is cleaved to regenerate biologically active PFT-α.

Acid-cleavable linkers are particularly preferred in the context of the invention. Osteoclastic bone resorption involves an acidic-mediated mechanism. At any given time, it is estimated that 15-20% of the bone surface is involved in resorption, formation, or mineralization (Kanis, Amer. J. Med., 91 (suppl SB), 29S (1991)). The pH at the bone surface during the osteoclastic bone resorption process has been measured using microelectrodes to be as low as pH 4.7 (Ghosh et al., J. Chem. Soc. Perkin Trans. 1, 8, 1964 (1979)). The delivery system of the invention provides not only a delivery site for drugs affecting the bone and bone marrow, but also provides a slow release reservoir site for drugs, including drugs administered systemically. By using acid labile protecting groups (e.g., acid-cleavable linkers) to mask the bone-targeted prodrug's activity, the invention further capitalizes on the bone resorption process as a prodrug activation mechanism. It has been demonstrated that small molecules (and ions such as calcium) liberated from the bone surface via osteoclasts are transported through the osteoclast to the extracellular space and can diffuse into nearby tissues, such as the bone marrow (see, for example, Stepensky et al., “Bone as an Effect Compartment: Models for Uptake and Release of Drugs” in Clinical Pharmacokinetics, 42(10), 863-881 (2003)).

Moreover, some diseases involving bone or bone marrow involve significant increases in bone remodeling resulting in increased resorption activity. The inventive method can be employed to deliver cell protection factors to the site of diseased bone to administer a cell protection factor to the bone marrow and surrounding tissues (e.g., muscle and connective tissue). Diseased bone sites provide a target environment for selective and enhanced activation of the prodrug at the particular site at which the drug is most needed. The active cell protection factor can function to save affected tissue or to prevent medical treatment-associated cell death of non-diseased tissue.

In view of the above, the invention provides a compound of Formula V:

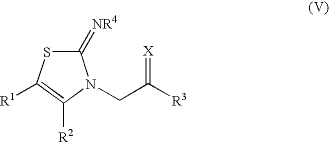

In the context of Formula V, R¹ and R² are taken together to form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties. R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, C₁-C₆ alkylamino, and/or C₄-C₁₄ aromatic or heteroaromatic moieties. X is Q, a carbonyl, or a protected carbonyl. R⁴ is hydrogen or an C₁-C₆ acyl group when X is Q, or R⁴ is Q when X is a carbonyl or protected carbonyl. Lastly, Q is an organic moiety that contains a nucleophilic or electrophilic reacting group and is cleavable under physiological conditions, thereby releasing a temporary p53 inhibitor.

By “electrophilic” is meant a reactive moiety that is electron deficient and can be reacted with an electron-rich compound. Examples of electrophilic reacting groups include, but are not limited to, acyl chloride, isothiocyanate, cyanate, isocyanate, bromoacetamide, acrylamide, maleimide, imidoester, acid anhydride, and activated ester. In one embodiment, compounds of the invention that contain Q having an electrophilic reacting group are reacted with an electron rich group of a bone targeting agent. By “nucleophilic” is meant a reactive moiety that is electron rich and could be reacted with an electron poor compound. Examples of nucleophilic reacting groups include, but are not limited to, hydroxyl, amino, thio, carboxyl, phosphono, sulfhydryl, semicarbazide, thiosemicarbazide, acylhydrozide, phenolate, and alkoxide. In one embodiment, compounds of the invention that contain Q having a nucleophilic reacting group are reacted with an electron poor group of a bone targeting agent. Alternatively, compounds of the invention containing Q having an electrophilic reacting group are reacted with a nucleophilic group of a bone targeting agent. The preferred reacting group will be determined by the particular application.

The invention also provides a compound of Formula VI:

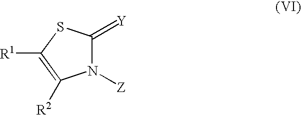

wherein R¹ and R² are taken together to form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties. Also with respect to Formula VI, R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. Y and Z, taken together, complete a 5-member imidazole ring of Formula VII or Formula VIII.

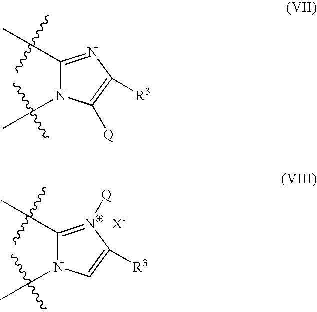

In Formula VIII, X⁻¹ is a counterion selected from the group consisting of a chloride, a bromide, a fluoride, an iodide, an acetate, a formate, a phosphate, a sulfate, and other pharmaceutically acceptable anions.

In one embodiment, Q of Formula VIII is —CH₂O—. Alternatively, Q of Formula VIII is A-J, wherein A is —CH₂O— and J is a bone targeting agent. The compound of the invention can be Formula XI:

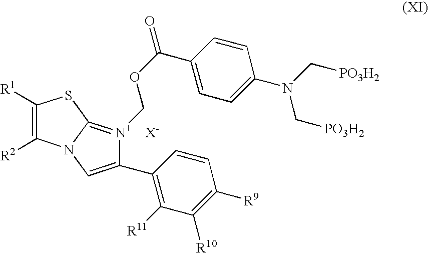

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R⁹, R¹⁰, and R¹¹ are each independently a hydro, methyl, fluoro, chloro, bromo, nitro, amino, methoxy, or phenyl moiety, and X⁻ is a counterion selected from the group consisting of a chloride, a bromide, a fluoride, an iodide, an acetate, a formate, a phosphate, a sulfate, and other pharmaceutically acceptable anions.

The invention further provides a compound of Formula IX:

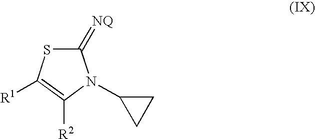

wherein R¹ and R² are taken together to form an aliphatic or aromatic carbocyclic 5- to 8-membered ring, optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties.

With respect to Formulas V-IX, Q is an organic moiety that contains a nucleophilic or electrophilic reacting group and is cleavable under physiological conditions. As used herein, Q is preferably cleavable under acidic physiological conditions, or is hydrolytically- or enzymatically-cleavable. Preferably, Q is an enol ether, ketal, imine, oxime, hydrazone, semicarbazone, acylimide, or methylene radical. Q can also be A-J, wherein A is an organic moiety that is cleavable under physiological conditions, and J is an organic moiety that selectively binds a cell or tissue (e.g., bone) in vivo. In this respect, R⁴ is hydrogen or an C₁-C₆ acyl group when X is A-J, or R⁴ is A-J when X is a carbonyl or protected carbonyl. A is an organic moiety that is cleavable under physiological conditions, and J is an organic moiety that specifically binds a cell or tissue in vivo. Preferably, A is an organic moiety that is cleavable under acidic physiological conditions, is hydrolytically cleavable under physiological conditions, or is enzymatically cleavable. J preferably is a bone targeting organic moiety, such as a bone targeting organic moiety selected from the group consisting of a bisphosphonate, a hydroxybisphosphonate, a phosphonate, a phosphate, an aminomethylenephosphonic acid, and an acidic peptide. Particularly preferred bone targeting agents (also referred to as bone targeting organic moieties) include alendronate, pamidronate, 4-aminobutylphosphonic acid, N,N,N,N-tetrakis-(phosphonomethyl)-ethylenediamine, 1-hydroxyethane-1,1-diphosphonic acid, phytic acid, N,N,N,N-tetrakis(methylphosphono)-1,5,8,12-tetraazacyclotetradecane, N,N-bis(methylphosphono)-4-amino-benzoic acid, nitrilotri(methylphosphonic acid), aspartyl hexapeptide, and glutamyl hexapeptide.

By “methylene radical” is meant an acid cleavable CH₂ group linking the cell protection factor to a bone seeking group. In one preferred embodiment, this methylene radical connects pifithrin-β via a quaternary amine as shown in Formula VIII to an acetoxy group. This embodiment is further depicted in the examples provided herein.

In another embodiment, the compound is that of Formula V, wherein X is a carbonyl and R⁴ is either Q, which is acid cleavable, or A, which is selected from the group consisting of 4-aminophthalic acid, succinic acid, 4-aminophenylacetic acid, and 4-aminobenzoic acid. For example, the compound can be that set forth in Formula XII:

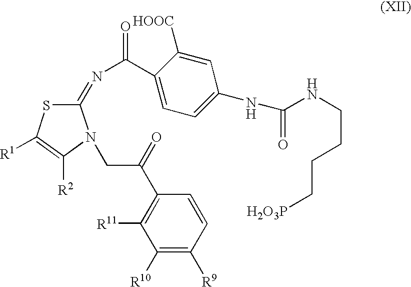

wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties. R⁹, R¹⁰, and R¹¹ are each independently a hydro, methyl, fluoro, chloro, bromo, nitro, amino, methoxy, or phenyl moiety.

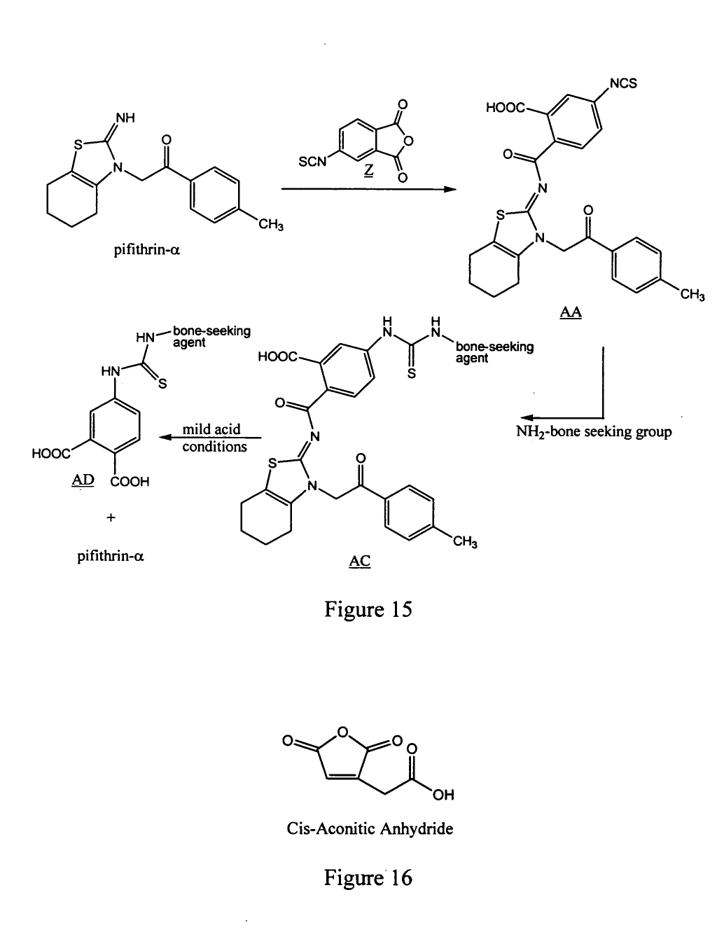

In some instances, the cell protection factors of the invention also can be covalently attached to bone-homing agents such as, but not limited to, monoclonal antibodies and proteins, wherein the cell protection factor is liberated from the bone in biologically active form over time. For example, in FIG. 15, compound [AA] can react with an antibody's amino or alcohol group to attach the cell protection factor to the antibody in a cleavable manner. The antibody can thus, deliver the prodrug to target tissue, where the biologically active cell protection factor is released.

The compounds of the invention may be formulated into various compositions, especially for administration to a mammal in, for example, therapeutic and prophylactic treatment methods. The compounds of the invention, including compounds that do not contain a bone targeting agent, can be used as prodrugs for a cell protection factor (e.g., pifithrin) having improved pharmacokinetic and/or bioavailability properties. A composition comprising the inventive compounds can be used to protect tissue from unwanted cell death caused by, for example, chemical or environmental insult. The composition is particularly useful in protecting bone marrow from toxicity associated with radiation and chemotherapy. The composition for use in the inventive method comprises one or more compounds described herein and a physiologically-acceptable (e.g., pharmaceutically-acceptable) carrier. Pharmaceutically-acceptable carriers are well-known to those who are skilled in the art, as are suitable methods of administration of such compositions to a mammal (e.g., a human). The choice of carrier will be determined in part by the particular inventive compound, as well as by the particular method used to administer the composition. If desired, the cell protection factors of the invention can be incorporated into nanoparticles for sustained release in vivo. Nanoparticles containing cell protection factors are further described in U.S. patent application Ser. No. ______ (Attorney Docket No. 224298), filed Apr. 2, 2004, and U.S. Provisional Patent Application No. 60/460,355, filed Apr. 3, 2003, which are hereby incorporated by reference in their entirety. Further, various routes of administering a composition to a mammal are available. Although more than one route may be available, a particular route of administration may provide a more immediate and more effective response in the mammal than another route.

Ideally, a compound of the invention (e.g., a cell protection factor covalently linked to a bone targeting agent via a physiologically-cleavable linker) is administered parenterally (e.g., subcutaneous, intramuscular, intracapsular, intraspinal, intrasternal, intravenous, or intraarterial administration). Formulations suitable for parenteral administration are well known in the art, and include aqueous and non-aqueous, isotonic sterile injection solutions, which may contain anti-oxidants, buffers, bacteriostats, and solutes that render the formulation isotonic with the blood of the mammal, and aqueous and non-aqueous sterile suspensions that may include suspending agents, solubilizers, thickening agents, stabilizers, and preservatives. Parenteral formulations can be presented in unit-dose or multi-dose sealed containers, such as ampules and vials, and may be stored in a freeze-dried (lyophilized) condition requiring only the addition of the sterile liquid carrier, for example, water, for injections, immediately prior to use. Extemporaneous injection solutions and suspensions may be prepared from sterile powders, granules, and tablets.

In one embodiment, a composition comprising the compound of the invention (e.g., a cell protection factor covalently attached to a bone targeting agent via a linkage cleavable under physiological conditions) is administered directly to the area surrounding bone. While such procedures are invasive, direct administration to bone or bone marrow can provide a more immediate effect than, for instance, intravenous administration. A surgical procedure similar to that for aspirating bone marrow can be performed to administer the inventive compound directly to bone marrow. Upon release of the cell protection factor from the bone targeting agent, the cell protection factor blocks the activity of, for example, p53 to protect the bone marrow from radiation- or chemotherapy-induced death. At least a portion of the inventive compound remains attached to the bone tissue via the bone targeting agent, which creates a sustained release mechanism of the cell protection factor to the bone marrow.

The compounds of the invention can be administered to other regions of the body containing calcium deposits for delivery of the cell protection factor to tissue suffering from or at risk of suffering from uncontrolled cell death. For example, a compound of the invention can be administered to an animal to inhibit cell death associated with ischemia, such as ischemia/reperfusion injury of the heart or limbs, wherein the ischemia is associated with calcium deposits in the vasculature (e.g., arterial calcification).

While not particularly preferred, a composition comprising the inventive compound can be introduced into a mammal via oral, nasal, topical, rectal, or vaginal administration. Formulations suitable for oral administration can comprise liquid solutions, such as an effective amount of the inventive compound dissolved in diluents, such as water, saline, or orange juice, as well as capsules, sachets or tablets, each containing a predetermined amount of the active ingredient. Oral formulations can be presented as solids or granules; solutions or suspensions in an aqueous liquid; and oil-in-water emulsions or water-in-oil emulsions. Tablet forms may include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers.

Aerosol formulations to be administered via inhalation can be incorporated into pressurized acceptable propellants, such as dichlorodifluoromethane, propane, nitrogen, and the like. Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, gels, and the like containing, in addition to the active ingredient, such carriers as are known in the art.

Formulations for rectal administration commonly comprise a suppository with a suitable base comprising, for example, cocoa butter or a salicylate. Formulations for vaginal delivery can comprise, for example, pessaries, tampons, creams, gels, pastes, foams, or spray formulas containing, in addition to the active ingredient, such carriers as are known in the art to be appropriate.

The appropriate dose of inventive compound administered to a mammal in accordance with the inventive method should be sufficient to affect the desired response in the mammal (e.g., a human) over a reasonable time frame. Dosage will depend upon a variety of factors, including the age, species, and size of the mammal, as well as the amount or length of exposure to the cell killing agent (e.g., chemical or environmental insult), if appropriate. Dosage also depends on the particular cell protection factor and bone targeting agent employed. The size of the dose also will be determined by the route, timing, and frequency of administration as well as the existence, nature, and extent of any adverse side effects that might accompany the administration of the inventive compound and the desired physiological effect. Some situations, such as exposure of a mammal to multiple rounds of chemotherapy or radiation therapy, may require prolonged treatment involving multiple administrations. The actual dose of inventive compound and schedule of administration to achieve an effective level of cell protection factor ideally achieves a blood or tissue level (e.g., 0.1-1000 nM) desired in the mammal that corresponds to a concentration of a cell protection factor that affects a desired level of cell protection (i.e., reduces or inhibits cell death to a desired degree) in an assay known to predict for in vivo activity of chemical compounds and biological agents.

The inventive compound can be packaged in unit dosage form, i.e., physically discrete units suitable as unitary dosages for a mammal, each unit containing a predetermined quantity of the inventive compound calculated in an amount sufficient to produce the desired effect in association with a pharmaceutically acceptable diluent, carrier, or vehicle. Unit dosage forms can be incorporated into a kit for inhibiting cell death in a mammal, wherein the compound of the invention is provided in combination with a physiologically-acceptable carrier and instructions for administration to a mammal.

One skilled in the art can easily determine the appropriate dose, schedule, and method of administration for the exact formulation of the composition being used, in order to achieve the desired effective concentration or amount of compound in the mammal. One skilled in the art also can readily determine and use an appropriate indicator of the effective concentration or amount of compound of the invention by a direct (e.g., analytical chemical analysis) or indirect (e.g., detection of apoptosis levels) analysis of appropriate patient samples (e.g., blood and/or tissues).

The compounds of the invention can be administered to a subject alone, or in combination with other pharmaceutically active compounds. Additional pharmaceutically-active compounds can be administered before, concurrently with, e.g., in combination with the compound of the invention in the same formulation or in separate formulations, or after administration of the compounds of the invention as described above. For example, factors that control inflammation, such as ibuprofen or steroids, can be co-administered to reduce swelling and inflammation associated with administration of the compounds of the invention. Similarly, vitamins and minerals, anti-oxidants, and micronutrients can be co-administered. Antibiotics, i.e., microbicides and fungicides, can be co-administered to reduce the risk of infection associated with administration of the compound of the invention. A compound of the invention can be used in combination with a cancer therapy, such as radiotherapy or chemotherapy. In particular, a compound of the invention can be used in conjunction with (e.g., co-administered with or prepared in a medicament comprising) chemotherapeutic drugs, such as adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, esperamicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, tauromustine, teniposide, thioguanine, thiotepa, Vinca alkaloids, vinblastine, vincristine, vinorelbine, paclitaxel, transplatinum, 5-fluorouracil, and the like.

The following examples further illustrate the invention but, of course, should not be construed as in any way limiting its scope.

### EXAMPLE 1

This example demonstrates a method of preparing pifithrin-α.

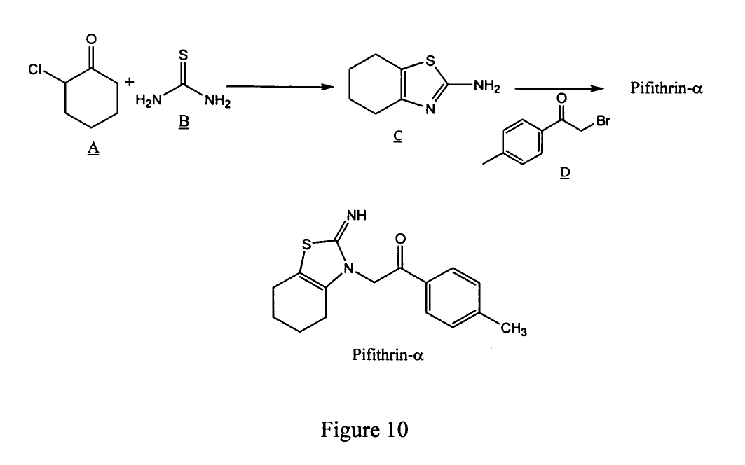

A multigram sample of pifithrin-α (the chemical structure of which is set forth in FIG. 1) suitable for the derivatization experiments, can be prepared according to literature methods as shown in FIG. 10 (see, for example, International Patent Application WO 00/44364; Tasaka et al., J. Heterocyclic Chem., 34, 1763 (1997); and Andreani et al., J. Med Chem., 38, 1090 (1995)). Referring to FIG. 10, the 2-aminothiazole (C) is prepared from the reacting cyclohexanone (A) with thiourea (C). Then the solvent is removed, and the solid is recrystalized from hexane. This sample (C) is then dissolved with a slight excess of commercially available p-methylphenacyl bromide in toluene and then stirred for 48 hours at room temperature, at which time PFT-α precipitates out of solution as the HBr salt. PFT-α can be converted into pifithrin-β simply by refluxing in methanol for 6 hours.

### EXAMPLE 2

This example demonstrates a method of making chemical modifications to pifithrin-α which allow for reversible covalent attachments.

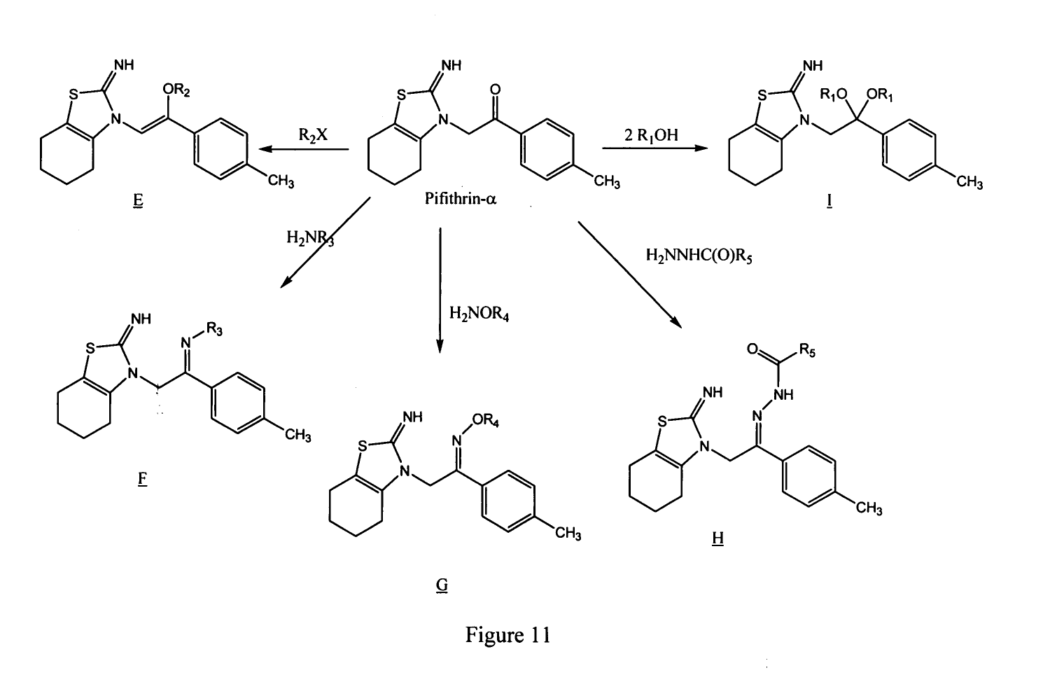

With the sample prepared in accordance with Example 1, a number of different chemistries can be utilized for reaction with this particular substituted beta-ketothiazole. These different methods are shown in FIG. 11. Referring to FIG. 11, the preparation of the enol ether derivative (E) (or enol acetate, R₂=acyl group) will be performed under basic conditions in the presence of an alkylating agent (or acylating agent) by known methods (Eroclspm et al., J. Org. Chem., 30, 1050 (1965)). The enol ether (and enol acetate) will then be evaluated for stability under relevant conditions. Relevant conditions for all of these conversions will be at either pH=7.4 (to simulate approximate physiological pH conditions) or pH=5 (to simulate bone resorption pH) in 50 mM sodium phosphate buffer (to simulate physiological conditions) at 37 degrees centigrade. These conditions can be employed to identify specific enol ethers (or enol acetates) for use as reversible/cleavable attachment functionalities (i.e., linkers) for ultimate attachment of bone-seeking groups.

The other four modifications of pifithrin-α shown in FIG. 11 rely on dehydration reaction or loss of water in their conversions. Thus, each is inherently susceptible to hydrolysis at some rate which would release intact pifithrin-α. Examples of each of the illustrated classes of compounds can be examined for cleavage potential under relevant conditions (pH=7.4 and pH=5 in 50 mM sodium phosphate buffer at 37 degrees centigrade).

Ketals, such as (I), can be prepared from alcohols under usual literature acidic conditions. Traditionally, ketals are not readily hydrolyzed back to ketones under mild physiological conditions. In this situation, however, the benzylic carbonyl group works to stabilize the intermediate benzylic carbocation resulting from the protonation of the ether linkage of the ketal under mild acidic conditions. Thioketals have been considered but are more difficult to cleave than oxygen acetals. Alkyl alcohols and diols (such as ethylene glycol and 1,3-propylenediol-containing compounds) also may be appropriate. One drawback to the ketal derivative approach is the requirement of an excess of alcohol (as solvent) to effect ketal conversion. Thus, achieving a high molar ratio of alcohol to ketone can be difficult. An additional drawback of the ketal derivative approach is the possible displacement of the imino group under ketal forming conditions.

Preparation of imines, such as (F), from primary amines can be accomplished under forcing conditions to drive off water via distillation (azeotropically) or chemically with a drying agent such as mole sieves (Rubottom et al., J. Org Chem., 48, 1550 (1983)) or titanium tetrachloride (Weingarten et al., J. Org. Chem., 32, 3246 (1967)). In general, when the amine is aliphatic, the resulting imines are relatively unstable in water. Use of an aromatic amine, such as aniline, forms a more stable version of imine termed a Schiff base. Both aliphatic and aromatic amines, to form imine derivatives of pifithrin-α, can be produced and tested for stability under relevant physiological-like conditions.

Oximes, such as (G), represent an attractive derivative because oximes can be readily hydrolyzed. The O-subsistent (R₄) of the hydroxylamine will ultimately bear the bone-seeking group and can be prepared synthetically by using a N-protected hydroxylamine to couple to the bone seeking group and then secondarily deprotecting the NH₂ group of the hydroxylamine in preparation for reaction with the carbonyl group of pifithrin-α.

Reacting pifithrin-α with a hydrazine or hydrazide can be performed to produce the hydrazone derivative, such as (H). Hydrazones (R₅=aromatic group) and more easily semicarbazones (R₅=—C(O)—NH₂) are quite susceptible to hydrolysis (March, J. Advanced Organic Chemistry, 4^{th} ed., 884 (1992)). A variety of commercially available hydrazines and hydrazides can be selected for their ability to react under acidic dehydrating conditions with pifithrin-α to form hydrazones. Those hydrazines and hydrazides that form hydrazones can be evaluated for the desired reversibility property using the methods described herein.

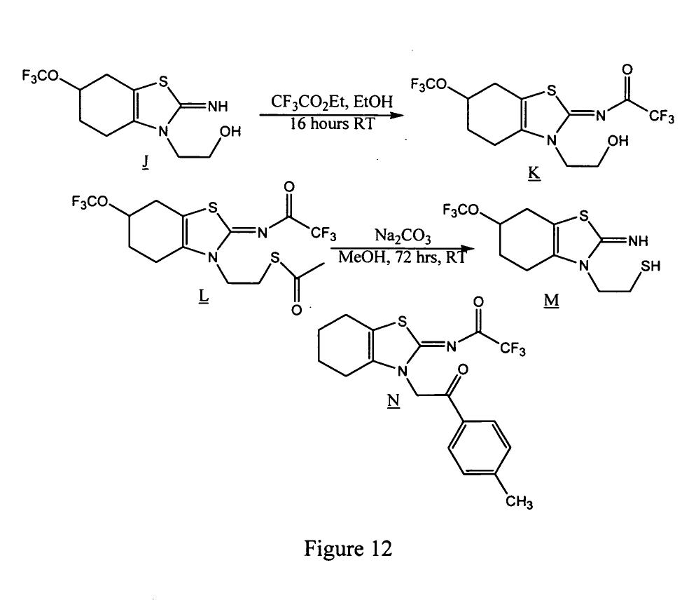

All of the above reactions to make derivatives of pifithrin-α focus on the carbonyl group. In that the imino-group of the imino-thiazole can be a reactive moiety as well, the NH of the imine group can be protected by using a trifluoroacetyl group. There is literature precedent for protection of such an NH group as shown in FIG. 12 (Jimonet et al., Heterocycles, 36(12), 2745 (1993)). Looking to FIG. 12, a close analog to pifithrin (J) was converted under the conditions shown in the presence of triethylamine to the trifluoroacetyl derivative (K) in 85% yield. The alcohol group of (K) was converted to a thiacetate using Mitunobu coupling to give (L). Removal of the protecting group was performed under mildly basic aqueous conditions to give a 74% yield of deprotected (M). Thus, an N-protected pifithrin derivative (N) can be generated based on the literature conditions for the close analog (J), the derivatization chemistries described above for PFT-α can be performed, and removal the trifluoroacetyl protecting group under mild basic conditions can be accomplished, which should not remove any of the reversible modifications (with the possible exception of the enol acetates).

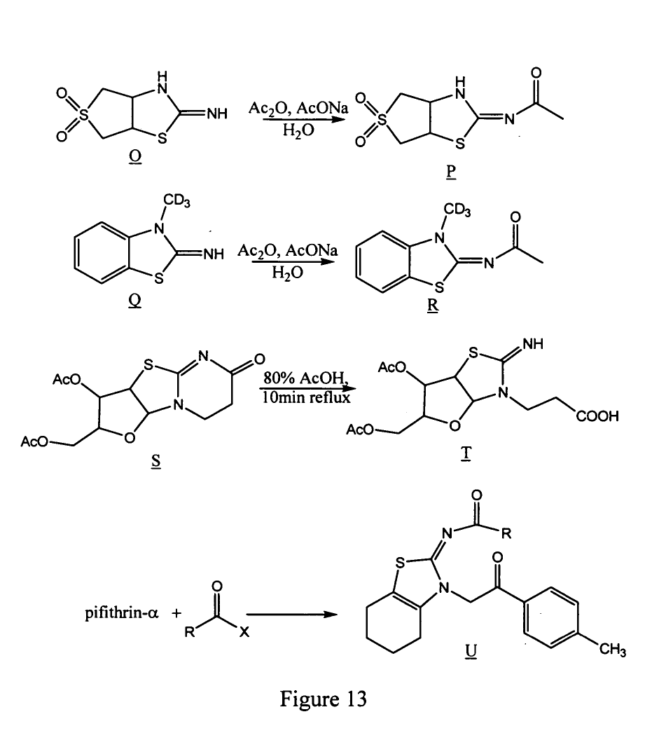

The facile acylation of the imine group described in the literature for (J) provides another avenue for reversible modification of pifithrin-α, namely by employing different acylating agents as the linker between pifithrin and bone seeking groups. The precedent for the acylation chemistry is shown in the conversion of (O) to (P) of FIG. 13 (Bezuglyi et al., Khim. Geterotsiki Soedin., 1, 110 (1998)) in 80% isolated yield and the conversion of (O) into (R) (Jagpolzomslo et al., Pol. Org. Mass. Spectrum., 24(10), 889 (1989)) in 85% isolated yield as depicted in FIG. 13. The hydrolysis of these acylated derivatives has not been reported. However, another acylated analogous compound (S) has been described to be completely deacylated with mild heat and acetic acid exposure to give (T) in 79% isolated yield (Skaric et al., J. J. Chem. Soc. Perkin Trans., 1, 779 (1985)). It should be noted that the acylated imine group of (S) was selectively hydrolyzed in a short time period in the presence of two esters which did not hydrolyze. This indicates that the acylated imine group is very susceptible to hydrolysis. Thus, preparation of acylated derivatives of PFT-α using standard anhydride chemistry or activated ester coupling chemistry to generate compounds of generic formula (U) may be possible. Moreover, (U) will hydrolyze back to the parent compound of FIG. 1. These derivatives can be made with various R groups (aliphatic, aromatic and bearing pendant groups that can be attached to the bone targeting groups described later).

All of the described synthetic methods can be efficiently performed using parallel reaction systems (such as Quest 210 apparatus, Radley Carosel Reactor, FlexChem System, etc.) to enhance productivity and to screen which compounds can be prepared while evaluating several different synthesis parameters. Products from successful reactions can be purified using a Shimadzu automated preparatory mass-triggered HPLC system. Preliminary studies indicate excellent mass spectrometer response for pifithrin-α using both APCI (atmospheric pressure chemical ionization) and ESI (electrospray ionization) modes.

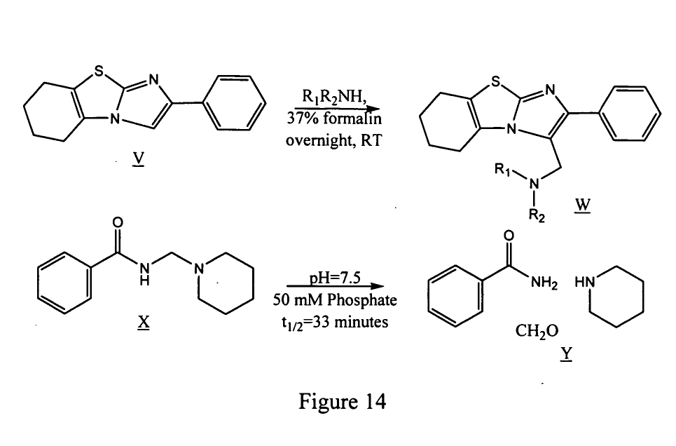

In some embodiments, pifithrin-β may be a preferred form of p53 inhibitor due to, for example, toxicity reasons. Pifithrin-β can be derivatized using Mannich base chemistry to prepare reversible derivatives suitable for attachment of bone-seeking groups. One report described the active 3-position of pifithrin-β analogs as suitable for Mannich reaction, as shown by the conversion of (V) to (W), as depicted in FIG. 14, under mildly acidic (10% AcOH in methanol) conditions overnight (Tasaka et al., Heterocyclic Chem., 34, 1763 (1997)). It should be noted that (V) differs from pifithrin-β by only a methyl group in the phenyl ring. It has been demonstrated that Mannich bases are often reversible under mild aqueous conditions, such as that found in blood serum. For example, the Mannich base, (X), prepared from benzamide, formaldehyde, and piperidine was found to rapidly decompose at neutral pH with a half-life of only 33 minutes (Johansen et al., Arch. Pharm. Chem. Sci. Ed., 9, 40 (1981)). Therefore, derivatives such as (W), where the R₁, R₂ groups serve to link the molecule to bone targeting groups, will be unstable Mannich-type bases in aqueous solution and will spontaneously hydrolyze back to pifithrin-β. Derivatives such as (W) will likely not be p53-inhibitors due to the large structural change, and will only exert p53 activity upon reversible cleavage at the bone surface. Such compounds are particularly preferred in the context of the invention. The p53 inhibitory bioactivity of such derivatives can be evaluated using methods described herein to confirm that the prodrug form of the derivative lacks activity. Bioactivity can be evaluated using two tests, the ConA cell test and the U87MG cell test, described herein.

### EXAMPLE 3

This example illustrates attachment of a cell protection factor, PFT-α, to a bone targeting agent via an acid-cleavable linker.

Preferably, the cell protection factor of the inventive compound is released from a bone targeting agent via an acid-enhanced cleavage mechanism. The acid sensitive linker ACL-3 (FIG. 6) is expected to acylate pifithrin-α (FIG. 1) on the imine NH moiety leaving the isothiocyanato group available to react with nucleophiles present on a bone-seeking moiety as shown in FIG. 15. This attachment through amines has been shown to be quite acid-sensitive (International Patent Application WO 94/00145). Given the facile hydrolysis of acylated imines, it can be expected that the acylated imine using ACL-3 would be even faster. This facile cleavage using the ACL-3 system is believed to occur because under acidic conditions the neighboring group effect of the ortho-carboxylate group facilitates cleavage. This chemistry is illustrated in FIG. 15. Reaction of PFT-α with ACL-3 (Z) in a normucleophilic but polar solvent, such as trifluoroethanol, is expected to acylate the imine to give (AA). The isothiocyanato group of (AA) is then expected to react with a nucleophilic group (e.g., an amino group) of the bone targeting agent (represented as (AB)) to generate the final drug conjugate (AC) for administration. Upon localization of (AC) in the bone, the compound will react under acidic conditions (such as bone resorption) to regenerate intact pifithrin-α, which is free to migrate away from bone and into the bone marrow to exert its p53-inhibitory effect. The other piece of the compound, (AD), is expected to remain on the bone surface, although under resorption conditions the bone seeking agent may be liberated from the surface. The toxicity of such low quantities of bone seeking agents is expected to be small to nonexistent based upon the amounts of bone seeking agents used in, for example, Quadramet (i.e., over 100 mg per injection) with no attendant short or long term toxicity attributable to the bone seeking part of the drug.

The ACL-3 acid cleavable linker was generated in two steps using thiophosgene to generate the isothiocyanate from the amine and then conversion of the diacid to the anhydride using forcing conditions with trifluoroacetic acid anhydride to give 744 mg (66% yield of >98% pure). PFT-α was acylated with this anhydride, and the isothiocyanide group was reacted with 4-aminobutylphosphonic acid to give the compound of FIG. 15 where the bone seeking group is —NHCH₂CH₂CH₂CH₂PO₃H₂, as characterized by LCMS. The acid cleavability of the resulting compound was not observed under the limited set of conditions tested.

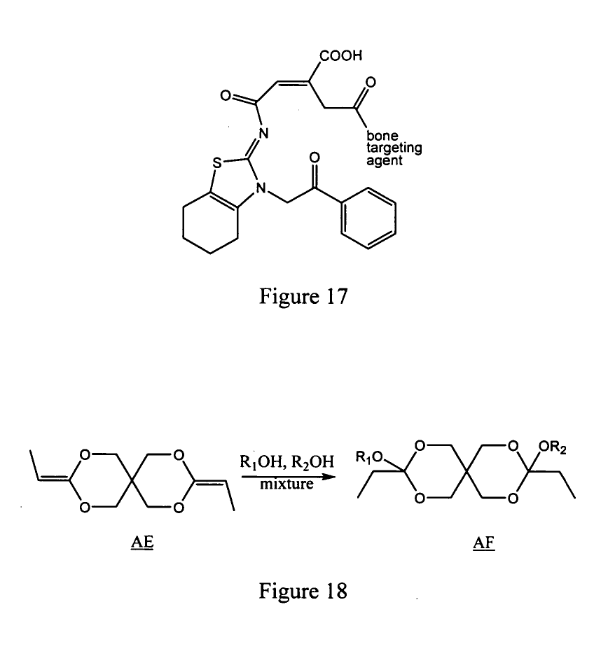

Another suitable acid cleavable linker system is aconitic acid. Unlike ACL-3, which can be synthesized in three steps from commercially available materials, aconitic acid is commercially available as the anhydride (cis-aconitic anhydride (the chemical structure of which is set forth in FIG. 16)) ready for coupling reactions. The product of amines acylated with cis-aconitic anhydride has been shown to have a half-life at pH 4 of only 3 hours (Shen et al., Biochem. Biophys. Res. Comm., 102(3), 1048 (1981)) hydrolyzing back to aconitic acid and free amine. Reacting the anhydride group of cis-aconitic anhydride with the imine NH of PFT-α generates a mixture of acylated products due to the asymmetrical nature of the anhydride. The isomers can be isolated chromatographically, and their separate cleavage rates to regenerate PFT-α can be determined using the methods described herein. The free carboxyl group (i.e., the most accessible aliphatic carboxylate group in cis-aconitic anhydride) can be attached to the bone targeting moiety via an amine or hydroxyl coupling reaction. This conjugate is illustrated by the structure set forth in FIG. 17, representing one of the isomers. Analogously to the chemistry described for the compound (AC) of FIG. 15, the compound of FIG. 17 is expected to localize in bone and release the active p53 pifithrin-α inhibitor upon exposure to mild acidic conditions (i.e., osteoclastic resorption).

A third suitable acid cleavable system utilizes an orthoester linkage (Guo et al., Bioconjugate Chem., 12, 291 (2001)). The third system uses (AE) (available from Advanced Polymer Systems) to react under acidic conditions with two different alcohol nucleophiles (R₁OH, R₂OH) as shown in FIG. 18 to generate a conjugate whereby the two alcohols are connected by an acid cleavable diortho ester linkage (AF). Alternatively, the conjugate can be generated using the enol form of the ketone moiety of pifithrin-α, which then would be trapped as an acid labile enol ether. It should be noted that there is no selectivity in the bisfunctional reagent (AE). The literature reports that the use of two different alcohols (R₁OH and R₂OH) results in a mix of reaction products with the reported yield of the desired product (e.g., two different alcohols attached) being a modest 20%. However, the desired product can be isolated by chromatography, as reported in Guo et al., Bioconjugate Chem., 12, 291 (2001). Subsequent exposure to acidic pH of 4 to 6 results in liberation of both the active p53 inhibitor and the bone seeking agent in alcohol form (i.e., liberate both R₁OH and R₂OH). Thus, PFT-α can be reacted for attachment through the enol tautomer in the presence of a bone seeking agent also possessing a nucleophilic group (e.g., an alcohol or an amine) to obtain a conjugate with an acid sensitive diortho ester linkage connecting PFT-A and the bone targeting group.

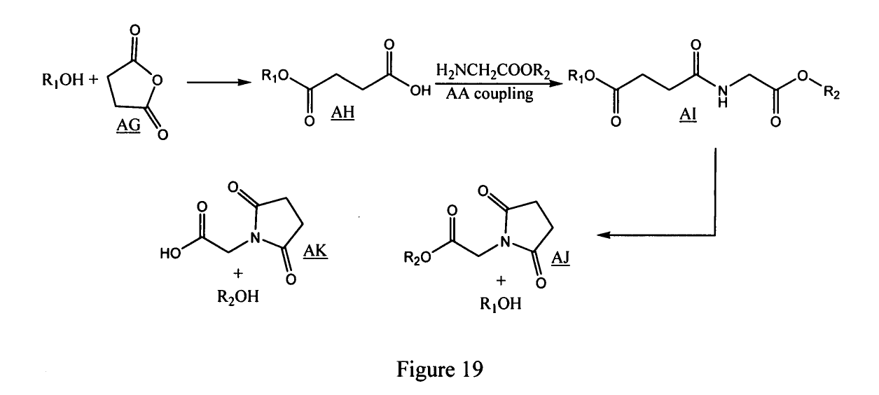

A fourth potential reversible conjugation system has been described in, for example, Matsumoto et al., Bioorgan. Med. Chem. Lett., 10, 1227 (2000). The fourth potential conjugation system capitalizes on the known instability of esters of succinamic acid to attach HIV-1 protease inhibitors to reverse transcriptase inhibitors, thereby creating a reversible conjugate (Bodanski et al., Int., J. Peptide Protein Res., 12, 69 (1978)). The chemistry is illustrated in FIG. 19 wherein R groups illustrate the principle (Matsumoto et al., supra). An alcohol (R₁OH) is reacted with succinic anhydride (AG) to generate the acid (AH). Acid (AH) is then coupled, using standard amino-acid carbodiimide chemistry, to generate the conjugate (AI). The nitrogen of the amide group in (AI) is predisposed to cyclize and cleave the ester, thus liberating R₁OH and giving the cyclized product (AJ). Ultimately, (AJ) is further hydrolized to yield (AK) and an alcohol R₂OH byproduct. The rates of these reactions can depend on steric effects and leaving group ability of R₁OH. Pifithrin-α is ideally trapped in its tautomeric enol form as the R₁OH group, and the bone targeting agents are represented by the R₂OH group.

The linker of the inventive compound preferably is reversibly attached to the cell protection factor and bone targeting agent functionalities to provide a range of stabilities at pH 7.4 and at acidic pHs. In that the compounds of the invention comprise major molecular modifications of the cell protection factor (e.g., pifithrin), the compounds, themselves, ideally possess no significant activity or, in the alternative, a level of p53-inhibiting activity which does not promote abnormal cell growth or cell protection in non-targeted tissues. The biological activity of the inventive compounds can be tested using the ConA cell line test and the U87MG cell line test described herein.

### EXAMPLE 4

This example illustrates the selection of bone targeting agents.

There are no adequate comparisons between bone targeting agents with respect to their affinity and ability to remain at the bone surface under standardized conditions. The affinity and retention of bone targeting agents, including commercially available bone targeting groups (e.g., phosphonates, aminophosphonates, bisphosphonates, hydroxydiphosphonates, and acidic polypeptides such as Asp₆ and Glu₆) can be determined as follows. Bone targeting agents can be exposed to hydroxyapatite, the uptake of candidate bone targeting agents quantitated, and the resistance of bone targeting agents to be washed off by successive washes can be determined. Other similar hydroxyapatite binding affinity assays have been reported (Fugisawa et al., Biochimisa et Biophysica Acta, 53, 1992 (1996)). All of these analyses can be carried out using LCMS as the analytical tool. The hydroxyapatite binding properties (e.g., adsorption rates, binding affinity, and wash-off rates) of candidate bone targeting agents can identify which bone targeting agents are optimal for use. For example, bone targeting agents demonstrating the fastest adsorption rates (e.g., to enhance localization from blood pool), highest binding affinity, and slowest wash-off rates (e.g., to minimize undesirable loss of conjugate from the bone) are particularly preferred in the context of the invention. Prolonged retention to bone may not be desired in some embodiments, in that the bone-avid portion of the inventive compound ideally is released from the bone and excreted upon cleavage of the p53 inhibitor to minimize long-term side-effects. Thus, bone residence time of the bone seeking agent desirably is longer than the time required for liberation of the p53 inhibitor.

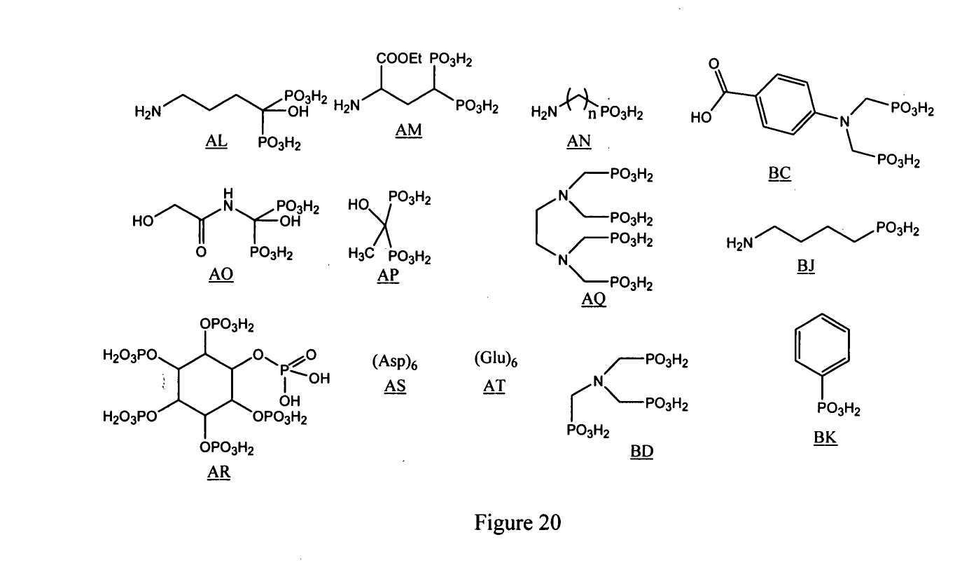

A noncomprehensive list of bone targeting agents for use in the inventive compound is provided in FIG. 20. All of the bone targeting agents of FIG. 20 possess a functional group that can be chemically modified for attachment to a cleavable linker or attached directly to a cell protection factor (e.g., a p53 inhibitor). One example of a suitable bone targeting agent is alendronte (AL) currently on the market as FDA approved Foxamax (Merck) for the treatment of osteoporosis. The toxicity profile of alendronte is well known, and the agent's documented ability to inhibit osteoclasts could be of additional utility in slowing diseases such as multiple myeloma, which appears with increased rates of bone resorption. The compounds of structures (AL), (AN), (AR), (AS), (AT), (AP), and (AQ) of FIG. 20 are commercially available. The bone targeting agent can be EDTMP (AQ), which previously has been injected intravenously in large quantities as part of the FDA-approved radioactive agent Quadramet. EDTMP is a preferred bone targeting agent for delivering the cell protection factor (e.g., p53 inhibitor) to bone due to its favorable toxicity profile. All of the compounds illustrated in FIG. 20, with the exception of (AQ), comprise an obvious nucleophilic group (e.g., a hydroxyl or an amine group) to participate in the chemical reactions described herein for conjugation to a cell protection factor through a cleavable linkage. To chemically attach a moiety to (AQ), one of the phosphonate hydroxyls is activated to form a phosphoramide or a phosphonate ester. Such a reaction also can be performed by making the bis-anhydride of (AQ). Likewise, (AM) (prepared as described in Sturtz et al., Eur J. Med Chem., 27, 825 (1992)) and (AO) (prepared as described in Fujisaki et al., J. Drug Targeting, 4, 117 (1994)) can be accessed using routine chemical synthesis reactions. Other hydroxyphosphonic acids can easily be generated by conversion from carboxylic acid using phosphorous acid and phosporous trichloride, examples of which are described in, for instance, Wingen et al., J. Cancer Res. Clin. Oncol., 111, 209 (1986).

Using the methods described herein, suitable bone targeting agents can be identified. For example, the bone targeting agent desirably exhibits the greatest hydroxyapatite uptake (quantity), fastest hydroxyapatite uptake (quickest sorption), and least demonstrated wash-off.

### EXAMPLE 5

This example demonstrates a method of covalently attaching a cell protection factor to a bone targeting group.

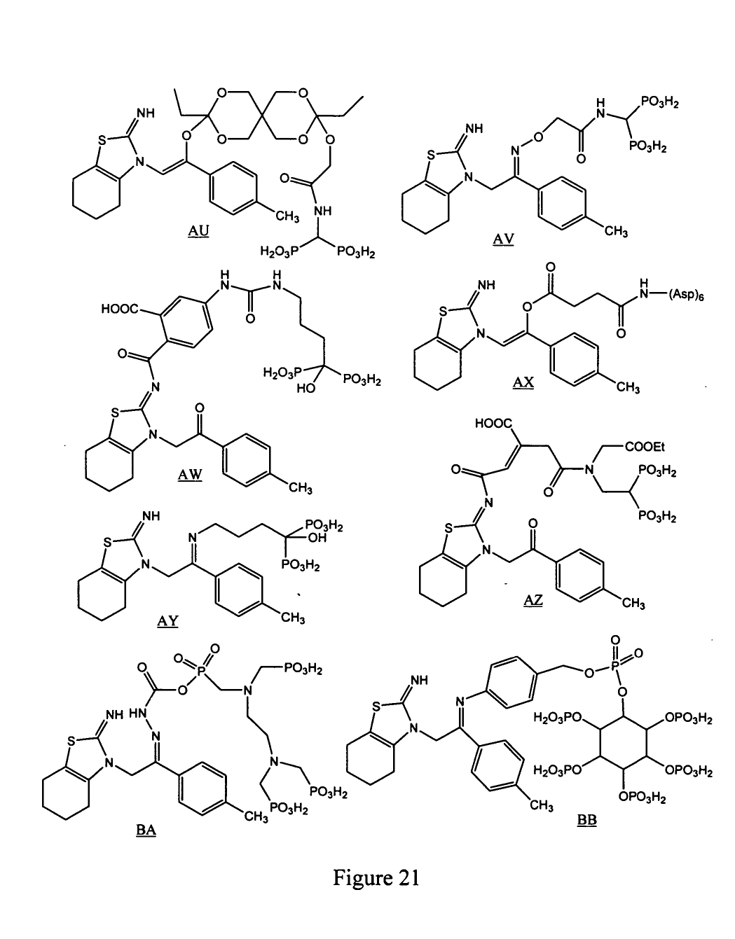

In creating compounds of the invention, cell protection factors (e.g., temporary p53 inhibitors) and bone targeting agents can be conjugated in a combinatorial fashion to make a multitude of combinations. For example, five suitable chemical transformations of pifithrin-α and five suitable bone targeting groups can be combined to generate 25 (5×5) conjugate products. Parallel synthesis and automated purification can be employed to generate numerous compounds in a short time period of time. Specific conjugates generated using the materials of Examples 1-4 are shown structurally in FIG. 21. Merely as an example of potential combinations, Table 1 lists specific attachment type, linkage, and bone targeting agents of each compound illustrated in FIG. 21:

TABLE 1
Potential Conjugates (all have pifithrin-α as base scaffold)

| | Linkage | Special Acid | Bone targeting |
|---|---|---|---|
| Conjugate | (structural type) | Cleavable | moiety |
| (see FIG. 21) | (see FIGS. 11 and 13) | Linkage Type | (see FIG. 20) |
| AU | enol ether (E) | AE (FIG. 18) | AO |
| AV | oxime (G) | none* | AO |
| AW | acylated imine (U) | | AL |
| AX | enol acetae (E) | AG (FIG. 19) | AS |
| AY | imine (F) | none* | AL |
| AZ | acylated imine (U) | | AM |
| BA | hydrazone (H) | none* | AQ |
| BB | imine (F) | none* | AR |

*“none” indicates that the linkage itself is expected to hydrolyze spontaneously over time at neutral pH

### EXAMPLE 6

This example describes methods of evaluating the reversibility parameters of the inventive compounds. The example also describes methods of verifying the bone affinity and lack of p53 inhibitory activity of the inventive compounds.

Candidate conjugates comprising a cell protection factor conjugated to a bone targeting agent via a cleavable linker can be evaluated for stability in physiological solution and interaction with hydroxyapatite. Compounds also can be tested for inhibition of p53 protein activity. The compounds of the invention ideally are not active with respect to inhibiting p53 (i.e., the cell protection factor is not active until release from the bone targeting agent). Protocols for evaluating the interaction of conjugates with hydroxyapatite are described in, for example, Fujisawa et al., Biochimica et Biophysica Acta, 53, 1292 (1996). Briefly, solutions of varying concentration of compound (e.g., 1 μM to 30 μM) are exposed to 100 μg of hydroxyapatite at 37 degrees C. for 1 hour. The solutions are then centrifuged, and the quantity of compound remaining in the supernatant is determined. Dissociation constants (Kd) and maximal binding amounts (bmax) can be calculated by a double reciprocal plot of unbound constructs versus bound constructs (see, for example, Fugisawa 1996, supra).

A suitable method for determining the stability of compounds can be performed in aqueous solution at 37 degrees C. at pH=7.4 and at pH=5. Additionally, the compound of the invention can be adsorbed onto hydroxyapatite, washed, and the remaining hydroxyapatite-bound construct studied for its stability at both pH=7.4 and pH=5. The method is performed as described above by centrifuging the hydroxyapatite-bound conjugates at various time points, and analyzing the supernatant for released cell protection factor (e.g., temporary p53 inhibitor, such as PFT-α). The amount of released cell protection factor (e.g., temporary p53 inhibitor) generated over time can be determined by HPLC-MS. This methodology will demonstrate that the cell protection factor (e.g., PFT-α) is released from the bone targeting agent, but the methodology does not prove that the released cell protection factor is biologically active. The biological activity of released cell protection factor (e.g., PFT-α) can be confirmed and quantified as set forth in Example 7. It is expected that, since conjugation of pifithrin to bone targeting agents requires major molecular modifications of pifithrin, the pifithrin-bone targeting agent conjugates will not possess any significant activity.

### EXAMPLE 7

This example describes methods of evaluating the p53-inhibitory activity of a cell protection factor upon release from of a cell protection factor-linker-bone targeting agent complex.

Candidate compounds can be evaluated for p53 inhibitory activity in vivo. An in vitro assay, which reasonably predicts p53 inhibitory activity in vivo, involves collection of the supernatant of hydroxyapatite-sorbed compounds of the invention, incubated over time at 37 degrees Celsius and pH=7.4. p53 inhibition properties can be guided by LCMS determined amounts. Another test which simulates the in situ resorption-mediated release of p53 inhibitor at low pH and shows that the released p53 inhibitor is in a form and concentration capable of inhibiting p53 is accomplished by (1) exposing hydroxyapatite-bound acid-cleavable compounds of the invention to a low pH (such as pH=5) for a brief period of time (e.g., 30 minutes) to mimic osteoclastic resorption conditions; (2) adjusting the pH to pH=7.4 to mimic extra-osteoclastic conditions; and (3) exposing the supernatant to the p53 inhibitor testing procedures.

The amount of temporary p53 inhibitor present in solution can be measured by LCMS. The amount of temporary p53 inhibitor able to penetrate into cells used in the assay can elucidate a dose-response relationship useful in determining an effective concentration of compound to achieve a desired effect.

The compounds of the invention also can be characterized using two established cell systems to study the effect of, for example, pifithrin-α derivatives or pifithrin, itself, on p53 function. An NIH 3T3 murine fibroblast cell line, ConA, is engineered to express β-galactosidase from a p53 promoter, which is easily quantitated by colorimetric determination using X-gal staining of cells (Komarova et al., Embo J, 16, 1391-400 (1997); obtained from A. Levine, Princeton University, NJ). Assay of p53 transcription involves measuring β-galactosidase under exposure of cells to ultraviolet radiation, etoposide exposure, or PTEN transduction. Control promoter constructs which are linked to the firefly luciferase reporter without p53 response elements, can be included for analysis of specific effects of a cell protection factor, e.g., pifithrin-α, on transcription by p53 versus, for example, NFkB, AP-1, Elk, TIMP-2, and the like.

In addition, the U87MG tumor cell line, which is p53 positive and/or engineered to express the E6 papillovirus protein resulting in the specific degradation of p53, can be employed to demonstrate the specificity of the cell protection factor in vitro. In particular, U87MG cells are transfected with the pGL2 plasmid containing mdm2luc, the upstream sequence of which comprises the second mdm2 promoter (exon1-intron1-exon2-intron2-exon3-TATA-luciferase). The plasmid construct contains two consensus p53 response elements. U87MG cells are cotransfected with a second plasmid, pCMVbgal, which encodes β-galactosidase as an internal control to adjust for transfection efficiency. A third plasmid construct is termed mdm2pvluc, which is similar to mdm2luc but is deleted of the p53 response elements and is composed of (exon2-intron2-exon3-TATA-luciferase). Plasmid mdm2pvluc is devoid of p53 DNA binding sites. Plasmid mdm2pvluc is used to establish specificity of mdm2 promoter luciferase response to p53 transcription. A positive control, the pcDNA3.1 plasmid, is included in all U87MG experiments to transient transfect wild type p53 into these cells. Transcription is quantitated using Promega luciferase assay and β-galactosidase is quantitated with Tropix-galacto-light kit according to the manufacturer's recommendations. p53 expression can be confirmed by Western blot analysis of corresponding cell lysates after quantitation of total protein using the Bradford assay.

In employing ConA and U87MG cell lines, a tissue culture transwell system can be utilized, such as Costar available from Corning, where U98MG or ConA cells are separated from the lower chamber by a 0.4 micron pore-rated filter. In the lower chamber, hydroxyapatite-conjugated compounds of the invention, such as pifithrin-α derivatives linked to bone targeting agents, are added. Acidification of the hydroxyapatite-conjugated compounds to different pH levels is then achieved using different buffer systems. Following acidification and release of the cell protection factor, e.g., pifithrin-α, into the supernatant, the pH is restored to a physiologic range, the transwell containing adherent U87MG cells is replaced, and biological assay for p53 transcription inhibition is performed. More specifically, U87MG or ConA cells growing on the top membrane of the transwell system are first transfected with pGL2mdm2 and pCMVbgal plasmids 48 hours prior to exposure to the cell protection factor, e.g., pifithrin-α derivative, cleaved from hydroxyapatite. At different time points after cell transfection in the presence of different concentrations of pifithrin-α, luciferase and β-galactosidase assays are performed to quantitate p53 transcription as a function of exposure to the liberated pifithrin-α. The target concentration for pifithrin-α is in the range of 1-20 μM (further discussed in Komarov et al., Science, 285, 1733 (1999)). This range can be the target cell protection factor concentration following cleavage into the supernatant for comparison to the parent compound (i.e., the cell protection factor not covalently linked to a bone targeting agent).

Finally, the effects of a cell protection factor, e.g., pifithrin-α, liberated from hydroxyapatite on radiation-induced apoptosis can be evaluated using a TUNEL assay, which allows quantitation of cells undergoing apoptosis. A cell protection factor in the context of the invention, following liberation from the bone targeting agent, blocks apoptosis only in p53 expressing cell lines.

### EXAMPLE 8

This example describes a method of generating pifithrin-β.

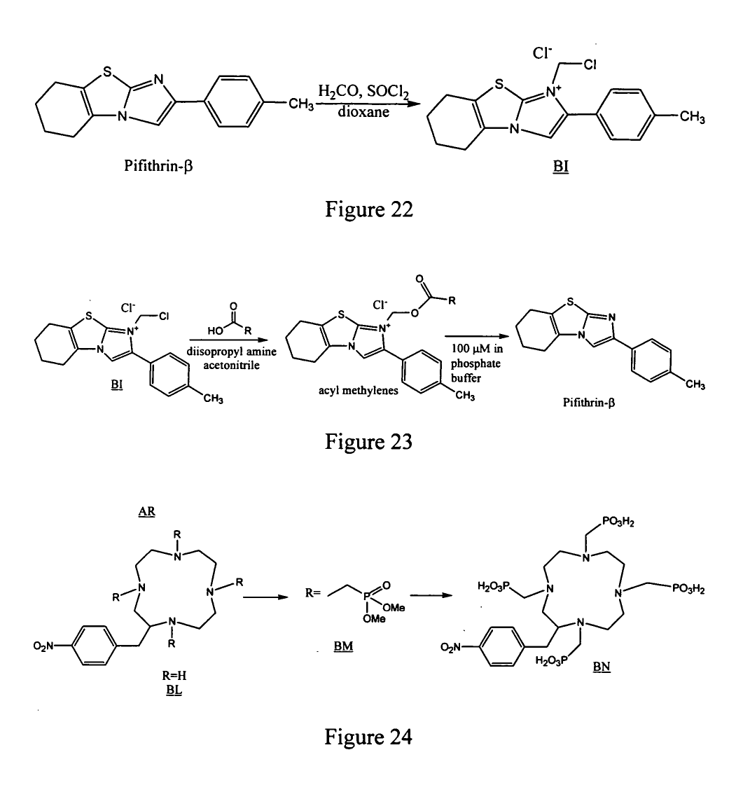

A prodrug strategy was devised using cyclized PFT-β and a structure activity relationship to obtain a prodrug linkage that converts to the native drug with about a 3 day half-life. A key novel intermediate (BI) (the chemical structure of which is set forth in FIG. 22) was generated in greater than 90% yield and >95% purity. Intermediate (BI) was found to react with even weak nucleophiles such as carboxylic anions as shown in the reaction scheme depicted in FIG. 23. This allowed preparation of the acyl methylene compounds recited in Table 2. All of the compounds of Table 2 were diluted into 50 mM phosphate buffer at pH=7.4 and pH=4.8 at room temperature and analyzed over time for stability. The amount of PFT-β was quantitated, and the amount of acyl methylene disappearing over time was quantitated by LCMS. A half-life for the cleavage of the acyl methylenes to generate PFT-β under relevant physiological conditions was determined. These half lives are set forth in Table 2 associated with the structures.

TABLE 2
Reversibility of Acyl Methylenes at 100 μM in Phosphate Buffer

| Chemical Structure of acyl | | | |
|---|---|---|---|
| methylenes | Designation | Half Life pH = 7.4 | Half Life pH = 4.8 |
| 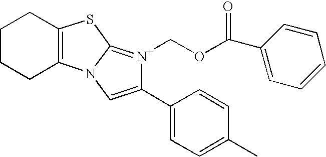 | BE | 72.8 hours | >500 hours |
| 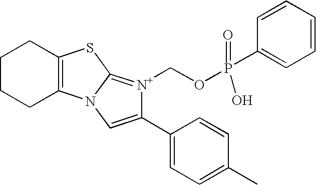 | BF | >500 hours | >500 hours |
| 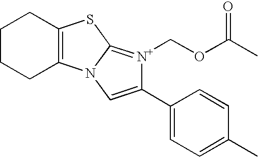 | BG | >500 hours | >500 hours |
| 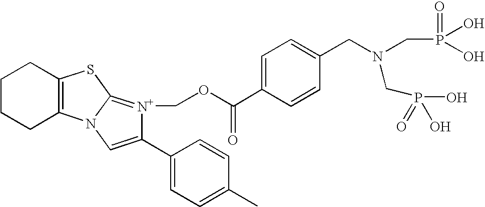 | BH | 77.4 hours | >500 hours |

Only the phenyl-substituted carboxylic acid quaternized methylene esters gave significant conversion of the prodrug back to the desired PFT-P. None of the compounds were found to be reversible at pH=4.8. It should be noted that the bone seeking groups designed and synthesized into (BH) did not materially effect the half-life of the useful quat methylene benzoic-acid ester linkage. This suggests that the cleavage rate of a particular linkage can be optimized without having to have the bone seeking groups present.

This example demonstrates the feasibility of generating the compounds of the invention, which are suitable for delivering a cell protection factor to the bone.

### EXAMPLE 9

This example describes the evaluation of bone targeting agents.

The bone seeking agents that we synthesized in multigram quantities are discussed herein (see FIG. 20) and include, for example, Alendronate (AL), which is used clinically as an anti-bone resorption drug; Quadramet (AQ); (BD), which is widely used in detergents; and (BC), which reacts specifically with chloromethyl quat derivatives of pifithrin such as PFT-β. (BJ), (AP), (AR), and (BK) were obtained from Sigma-Aldrich. The more complicated (BN), the chemical structure of which is depicted in FIG. 24, was prepared in small quantities from the commercially available (BL), which was phosphonomethylated to give the octa-methyl ester, (BM), which was then deprotected to give (BN) using trimethylsilyl bromide. (BN) is an analog of the best bone seeking agent known in the literature, DOTMP. DOTMP is in Phase III human clinical trials as a targeting agent for short lived radioisotopes for bone marrow ablation in treating multiple myeloma.

To determine the affinity of the bone targeting agents to hydroxyapatite, the method described in Willson et al., Biorganic & Medicinal Chemistry Letters, 6(9), 1043-1046 (1996) for determining bone affinity using HPLC retention on a hydroxyapatite column was modified. The affinity of several bone targeting agents was determined using the following method. A hydroxyapatite (HA) suspension was prepared by weighing BioRad CHT Type I 80 μm HA and adding Tris-buffered saline (150 mM NaCl, 50 mM Tris-HCl pH 7.40 [TBS]) at a concentration of 10.0 mg/ml. The suspension was diluted to prepare a 1.00 mg/ml HA suspension by stirring on a magnetic stir plate at the slowest speed necessary to create a uniform suspension appropriate for sampling. Each tested compound was dissolved in TBS at a concentration of 0.100 mM. 200 μl of test solution was mixed with 200 μl of TBS, HA at 1.00 mg/ml, or HA at 10.0 mg/ml, and agitated on an orbital shaker at 200 rpm for 1 hour. Each experiment was performed in triplicate. The supernatant solution was analyzed using single ion monitoring (SIM) mass spectral mode on a Shimadzu LCMS-2010 liquid chromatograph-mass spectrometer. The SIM counts in the test compound/TBS solutions were set at 100%. The amount of test compound present in the HA supernatant test solutions was expressed as a percentage of the amount of test compound available with no hydroxyapatite present. Tetracycline was included as a positive control.

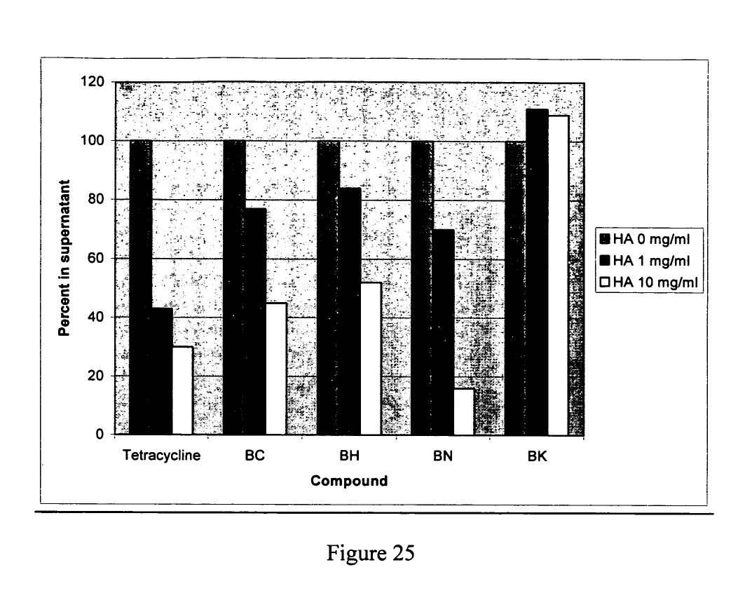

The results of these experiments are set forth in bar graph form in FIG. 25. As is apparent from the results, tetracycline has an affinity toward hydroxyapatite. An increasing amount of tetracycline is pulled out of solution as the amount of hydroxyapatite increases. Phenyphosphonic acid ((BK) of FIG. 20) has no observable affinity to hydroxyapatite. The analog of the best bone targeting agent known in the literature, (BN) of FIG. 20, demonstrates the best affinity toward hydroxyapatite in this assay. The affinity of synthesized bone affinity agent (BC) of FIG. 20 for hydroxyapatite was less than (BN) but very similar to tetracycline and vastly improved relative to the monophosphonic acid (BK). Likewise, the pifithrin prodrug conjugate (BH) has demonstrated affinity for hydroxyapatite almost identical to that of (BC) indicating that the addition of the large organic group (pifithrin) to the bone targeting group did not diminish significantly its affinity for hydroxyapatite. It should be noted that the screening method described herein may not be appropriate for all bone targeting agents due to the difficulty in quantitating the concentration of bone targeting agent in the presence of the tris-buffering agent, which appears to suppress the ionization of some bone targeting agents, thereby creating difficulty in using electrospray mass spectroscopy. However, other screening methods described herein and known in the art can be employed in such situations.

This example describes a relevant assay for evaluating bone targeting agents. Furthermore, this example demonstrated that a designed bone targeting agent, (BC), can be attached to pifithrin to generate (BH) and still retain similar bone affinity.

### EXAMPLE 10

This example describes a method of assaying a compound of the invention for release of a cell protection factor following binding to hydroxyapatite.

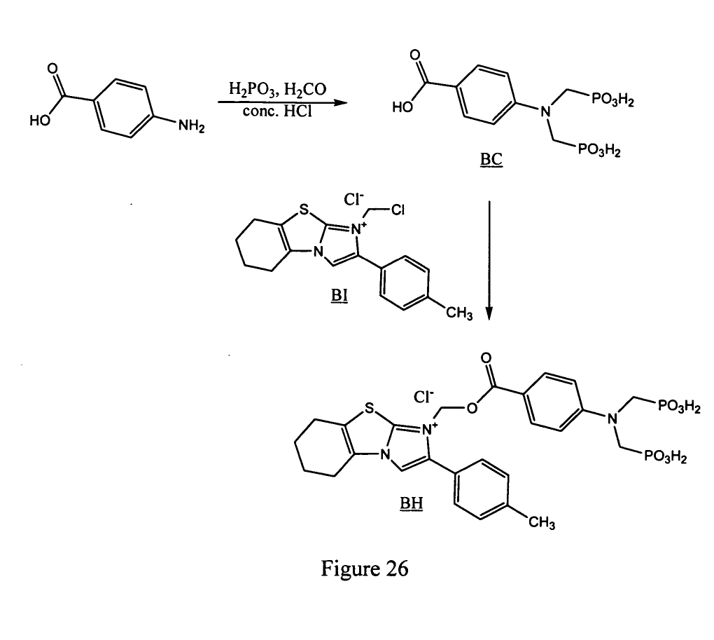

The final conjugate (BH) (see Table 2) was synthesized (assembled) as described and purified by HPLC. The overall synthesis is shown in FIG. 26. Its affinity to hydroxyapatite was demonstrated as described above in comparative testing. The ability of (BH) to bind to hydroxyapatite and to liberate PFN-β over time was demonstrated as follows. A 900 μL aliquot of 1.9 mM (BH) in 5 mM phosphate buffer at pH=7 was mixed with 100 μL of an hydroxyapatite suspension (25 weight percent hydroxyapatite; Sigma) containing approximately 25 mg of hydroxyapatite. The suspension was mixed gently for 15 minutes and then centrifuged at 10,000 rpm for one minute. A 100 μL aliquot was removed and analyzed by electrospray LCMS and compared to a standard solution that had not been exposed to hydroxyapatite. This comparison indicated that 77% of the originally present (BH) was bound to the hydroxyapatite solid (i.e., 23% of (BH) remained in the supernatant solution). The supernatant was decanted from the hydroxyapatite solid, an additional 900 μL of de-ionized water was added, and the mix was shaken vigorously for 20 seconds. The suspension was then centrifuged at 10,000 rpm for one minute. A 100 μL aliquot of suspension was removed and analyzed by electrospray LCMS and compared to the standard solution that had not been exposed to hydroxyapatite. This comparison indicated that only 3.5% of the originally present (BH) was in the supernatant (wash number 1). The supernatant was decanted from the hydroxyapatite solid, an additional 900 μL of de-ionized water was added, and the mix was shaken vigorously for 20 seconds. The suspension was then centrifuged at 10,000 rpm for one minute. A 100 μL aliquot was removed and analyzed by electrospray LCMS and compared to the standard solution. This comparison indicated that only 0.6% of the originally present (BH) was in the supernatant (wash number 2). Thus, of the original 1.74 [Moles of (BH) present in the supernatant and exposed to the hydroxyapatite, a total of 0.48 μMoles was recovered in the original supernatant plus the two water washes. This result implies that 1.26 μMoles of (BH) was adsorbed to the hydroxyapatite sample in such a manner as to resist washing off.

The hydroxyapatite of the suspension was then resuspended in wash number 2 supernatant by pipetting up and down. A 20 μL aliquot of the resulting 900 μL suspension was removed and extracted with methanol. An analysis of the methanol extract by LCMS (versus a standard of (BH)) indicated no detectable (BH) was extractable from the hydroxyapatite sample known to contain the 1.26 μMoles of (BH) described above. A small amount of the prodrug cleavage product, PFT-β (i.e., <7% of 1.26 μMoles known to be sorbed on the surface), was found to be extractable. The suspension was then gently rocked for 34 hours. A 20 μL aliquot was removed and analyzed after extraction with methanol. This analysis indicated no detectable extractable (BH), but the amount of prodrug cleavage product, PFT-β, had increased to 19.6% of the 1.26 μMoles of prodrug calculated to be sorbed on the hydroxyapatite. Another aliquot of the gently mixed suspension obtained after a total 105 hours (i.e., an additional 71 hours after the 34 hour datapoint) showed no detectable extractable (BH), but the amount of prodrug cleavage product, PFT-β, had increased to 29.1% of the 1.26 μMoles of prodrug calculated to be sorbed on the hydroxyapatite. An addition aliquot of the gently mixed suspension was obtained after a total 153 hours contained no detectable, extractable (BH). The amount of prodrug cleavage product, PFT-β, had increased to 37.5% of the 1.26 μMoles of prodrug calculated to be sorbed on the hydroxyapatite.

The whole remainder of the sample was extracted with 4 mL of methanol. The sample was filtered through a 0.2 micron syringe filter into a glass vial, and blown down with a gentle stream of argon to low volume leaving less than a milliliter of aqueous liquid. The aqueous sample was then lyophilized. The vial's contents were then redissolved in methanol. The vial was rinsed with methanol. The sample was transferred to a cone-bottomed vial and gently blown down to dryness with argon. The final material was dissolved in 30 μL of DMSO. A 2.0 μL aliquot of this solution was diluted into methanol, analyzed by LCMS, and compared to a standard of PFT-β. The recovered sample was found to be highly pure (>95% purity by LC) with identical retention time and mass spectrometry as a PFT-β standard. A comparison of peak areas of the PFT-β recovered from extraction of the prodrug bound to hydroxyapatite to the standard PFT-β indicated that the concentration of the recovered PFT-β in DMSO solution was 5.11 μMolar. This sample was demonstrated to be biologically active as described in Example 11.

The results of this example demonstrated the release of a cell protection factor from a compound of the invention following binding of the compound to hydroxyapatite.

### EXAMPLE 11

This example describes a method of evaluating the biological activity of a cell protection factor.

The binding to hydroxyapatite to the bone targeted prodrug (BH) and its conversion over time to liberate PFT-β was described in Example 10. The PFT-β liberated (5.11 millimolar solution from Example 10) was assayed for its biological activity by evaluating the degree of p53-mediated transcription in the presence and absence of the liberated PFT-β. The assay comprised transiently transfecting human brain endothelial cells (HBECs) with a plasmid comprising the MASPIN promoter, which contains two p53 binding sites, operably linked to the luciferase gene. A group of HBECs was transiently transfected with a plasmid encoding wild type p53. Thus, if p53 produced in the transfected HBECs is active, transcription of the luciferase gene will be activated and the produced luciferase detected. p53-mediated transcription is measured relative to the internal control plasmid pCMV-galactosidase to control for transfection efficiency.

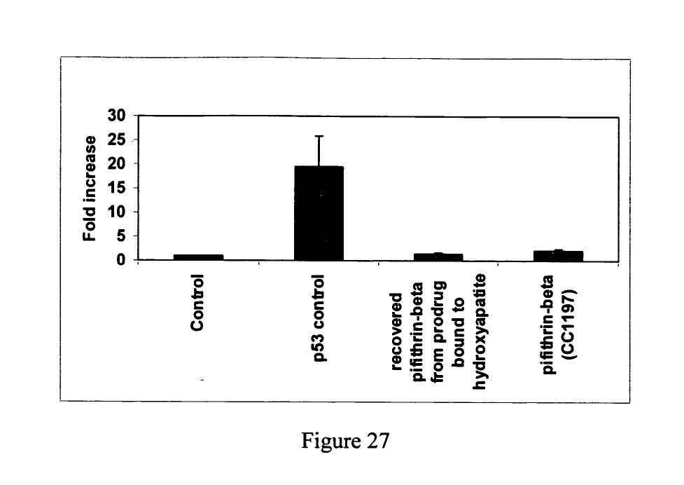

The results of the assay are set forth in the bar graph of FIG. 27, the data of which represents fold induction of p53-specific transcriptional activity in HBECs. Column 1 of FIG. 27 represents a negative control resulting from HBECs not transfected with the wild type p53 plasmid. Column 2 of FIG. 27 represents data obtained from HBECs transfected with the wild type p53 plasmid which were not exposed to PFT-β. Column 3 represents data from HBECs transfected with the wild type p53 plasmid in the presence of the liberated PFT-β of Example 10. The data represented by column 3 demonstrates that the pifithrin released from the prodrug (BH) attached to hydroxyapatite is biologically active. Column 4 represents data obtained from HBECs transfected with the wild type p53 plasmid in the presence of standard PFT-β compound (20 μM).

The data provided by this example illustrates targeting of a cell protection factor, e.g., pifithrin, to hydroxyapatite, a component of bone. The pifithrin was released from the prodrug (i.e., the cell protection factor covalently attached to a bone targeting agent via a physiologically-cleavable linker) over time. The pifithrin released was demonstrated to be bioactive by inhibiting p53-mediated transcription.

### EXAMPLE 12

This example demonstrates a method of generating pifithrin-α.

2-chlorohexanone (Aldrich) (20.01 g (151 mMole)) was dissolved in 300 mL of toluene. To the mixture was added 11.87 g of thiourea (Aldrich) in portions over a 2 minute period. The reaction mixture was heated to about 90° C. for 16 hours and allowed to cool to room temperature. The resulting precipitate was separated from the supernatant, washed with toluene, and dried to yield 12.07 g of a yellow solid. This material was dissolved in about 80 mL of water and basified with potassium carbonate to pH=9. The resulting cloudy mixture was extracted with methylene chloride. The organic layer was dried over sodium sulfate. The organic solvent was evaporated under vacuum to yield 3.06 g of brown oil. A 2.93 g (19 mMole) portion of the oil was dissolved in 30 mL of toluene and treated with a solution of 4.05 g of 2-bromo-4′-methyl-acetophenone (19 mMole) (Aldrich) in 20 mL of toluene. The reaction mixture was allowed to stir for 17 hours at room temperature. The resulting solid was filtered and washed with toluene to give 4.847 g (69% yield) of PFT-α as the hydrobromide salt. The free base of PFT-α was obtained by treating 349 mg of PFT-α hydrobromide salt with 1 M NaOH (15 mL) and chloroform (15 mL). The mix was stirred for 5 min, during which time the solid PFT-α free-base reacted and dissolved. The layers were separated and the organics dried over sodium sulfate and the solvent removed to provide 270 mg (99% yield) of a tan solid. PFT-α was characterized by electrospray HPLC-MS. giving a retention time of 1.8 minutes with the expected [M+H]+ for calculated [M=C₁₆H₁₈N₂OS] observed at m/z 287.

HPLC analysis was performed on a Shimadzu LCMS-2010 and employed a flow rate of 3 mL/min and a starting concentration of “B” solvent of 5%. The B solvent was linearly ramped to 95% concentration at 5.0 minutes, held at 95% until 6.0 minutes, then linearly ramped back down to 5% at 6.5 minutes, where it remained until the end of the run at 7.5 minutes. In addition to mass detection, the LC detection consisted of 3 channels: UV absorbance at 254 nm, UV absorbance at 214 nm, and evaporative light scattering (Alltech ELSD 2000). The evaporative light scattering detector was run at 50° C. with a nitrogen flow of 1.5 liters per minute. The cone desolvation line (CDL) and block temperatures of the Shimadzu LCMS-2010 were both 300° C., and the nitrogen nebulizer gas flow was 4.5 L/min. Positive and negative mass spectra were detected from 50 to 2000 m/z. A YMC CombiScreen ODS-AQ column was employed having a S-5 μm particle size, length of 50 mm, and a 4.6 mm I.D. Mobile phase “A” was made using HPLC grade B&J water with 0.1% (v/v) HOAc. Mobile phase “B” was HPLC grade B&J acetonitrile with 0.1% (v/v) HOAc. This system allows a retention time of 1.50 to 1.60 minutes for a standard commercially available material (4-hydroxyphenylacetic acid; Aldrich Catalog H5000-4; melting point 149-151° C.) used as a reference standard.

Gradient preparative HPLC was performed on a Shimadzu system composed of two LC-8A pumps connected to a SIL-10A autosample. Elution was performed over a reverse phase column (YMC, cat. CCAQSOSO52OWT; ODS-AQ CombiPrep, 20 mm×50 mm), and passed through an MRA variable volume splitter. The smaller stream was made up to 3 mL/minute using a LC-10ADVP make-up pump (MeOH). The eluent passed through a variable two channel wavelength UV detector and split roughly 6:1 to an evaporative light scattering detector (run at 50° C. with a nitrogen flow of 1.5 liters per minute) and a Shimadzu 2010 Mass detector. The larger stream from the MRA splitter then flowed to a Gilson 215 liquid handler serving as a fraction collector triggered by mass, UV absorbance, or ELS peak size.

Generally, different gradients were run starting with the more aqueous solvent “A” and ramping up to various concentrations of “B.” Mobile phase “A” was made using HPLC grade B&J water with 0.1% (v/v) HOAc. Mobile phase “B” was HPLC grade B&J acetonitrile with 0.1% (v/v) HOAc.

The resulting PFT-α demonstrated a retention time of t_{R}=1.8 minutes with the expected [M+H]+for calculated [M=Cl₆H₁₈N₂OS] observed at m/z 287.

### EXAMPLE 13

This example demonstrates a method of generating pifithrin-β, which is a modification of the procedure described in Tasaka et al., J. Heterocyclic Chem., 34, 1763 (1997).

Briefly, 15 g (113 mMoles) of 2-chlorocyclohexanone (Aldrich) was diluted in 150 mL of toluene and treated with 1.1 equivalents of thiourea (9.46 g, 124 mMol) (Aldrich) and 1.1 equivalents of triethylamine (12.54 g, 124 mMole). The mixture was heated at 95° C. overnight. A 34.8 g portion of 1.3 equivalents of 2-bromo-4′-methylacetophenone (Aldrich) was added (see FIG. 10). The mixture was stirred overnight to produce a tan solid. The tan solid was filtered and washed with toluene. The solid was taken up in chloroform and 10% (wt/wt) potassium bicarbonate, and stirred 5 minutes resulting in dissolution of the solid. The resulting layers were separated, the aqueous layer extracted with chloroform, and the combined organic layers were washed with 5% (wt/wt) potassium carbonate. The organic solution was dried over sodium sulfate. The solvent was removed to give a brown solid. The solid was subjected to silica gel column chromatography using 80/20 dichloromethane/methanol.

Fractions were recovered containing PFT-α and PFT-β. These fractions were dissolved in dioxane and heated at 90° C. overnight. Much solid precipitated upon cooling. After filtration and washing with diethyl ether, 3.97 g of pale yellow solid was obtained, representing a 13.1% overall yield of PFT-β. This material was characterized by LC-MS (the methods of which is described in Example 11) to give a retention time of 3.9 minutes and showed the desired mass of [M+H]+=269 m/z. The generated PFT-β demonstrated an identical retention time and mass spectral ionization compared to commercially available material (Cal-BioChem; catalog number 506134). A few milligrams of the generated PFT-β were dissolved in deuterated chloroform and the proton NMR spectrum obtained, which was consistent with the desired compound: 1H (CDCl₃): 7.71 (d, 2H, J 8.1 Hz), 7.51 (s, 1H), 7.20 (d, 2H, J 8.3 Hz), 2.72-2.64 (m, 4H), 2.37 (s, 3H), 1.99-1.92 (m, 4H).

### EXAMPLE 14

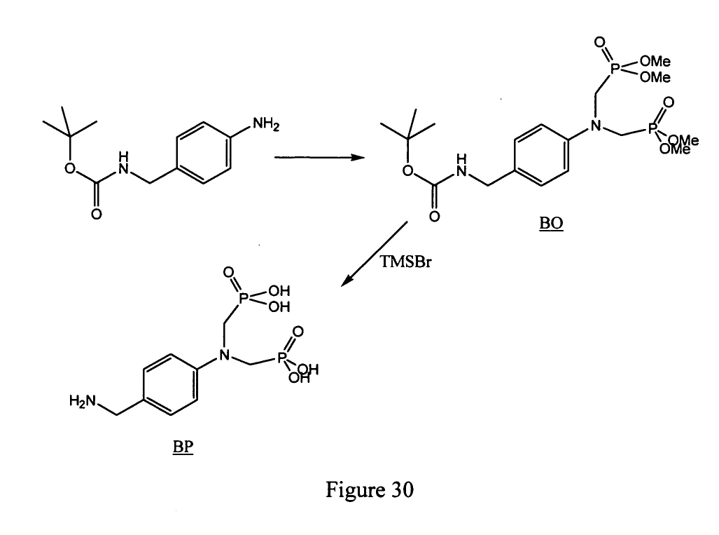

This example demonstrates a method of preparing a bone targeting group, the chemical reaction of which is illustrated in FIG. 30.

A solution of 500 mg 4-[(N-BOC)aminoethyl]aniline (Aldrich) in 10 mL dioxane was treated with paraformaldehye (400 mol %, 270 mg) and trimethylphosphite (400 mol %, 1.12 g). The mixture was heated to 95° C. overnight. Additional paraformaldehyde (270 mg) and trimethylphosphite (1.12 g) were added, and the resulting mixture was heated at 95° C. overnight. The solution was cooled, taken up in chloroform (20 mL), and washed with saturated sodium chloride (20 mL) and water (20 mL). The organics were dried over sodium sulfate. The solvent and excess trimethylphosphite was removed via rotary evaporation at 80° C. to provide 1.723 g of a clear oil. The presence of compound (BO) (as designated in FIG. 30) was confirmed by electrospray HPLC-MS (methods described in Example 12) showing a retention time of t_{R}=2.9 minutes and a mass of 467 m/z [M+H]+ and 489 m/z [M+Na]+ found for the desired mass [M=C₁₈H₃₂N₂O₈P₂].

A solution of 870 mg of (BO) (as designated in Figure) in 10 mL dichloromethane was treated with bromotrimethylsilane (690 mol %, 1.97 g). The solution was stirred overnight. Methanol (10 mL) was added and the solution was stirred 15 min. The solution was concentrated to provide 1.12 g of an orange oil. The presence of (BP) was confirmed by electrospray LC-MS using a special slow gradient. To achieve a slow gradient for polar compounds, a flow rate of 3 ml/min and a starting “B” concentration of 0% was employed. These conditions were held for the first minute, after which the “B” solvent was linearly ramped to 10% concentration at 3.0 minutes, then linearly ramped to 95% at 5.0 minutes, where it was held until 6.0 minutes. The sample was linearly ramped to 5% at 6.5 minutes, where it remained until the end of the run at 7.5 minutes. The retention time using this gradient was found to be t_{R}=0.85 minutes and the mass spectrometry for the desired product [M=C₉H₁₆N₂O₆P₂] found at the expected m/z 309 [M−H]⁻ operating in the negative mode.

### EXAMPLE 15

The conversion of PFT-α to PFT-β in human blood and buffer is described in this example.

A 10.0 mMolar solution of PFT-α in DMSO was prepared. A 5 μL aliquot of the solution was diluted with 495 μL of methanol, and further diluted with 500 μL of acetonitrile to produce a comparison standard 50 μMolar solution. Five 5 μL aliquots of 10.0 mMolar solution of PFT-α in DMSO was added to each of five plastic vials containing 495 μL of human serum (Sigma-Aldrich), and the vials were capped. The solution was mixed thoroughly and allowed to stand at 37° C. At different timepoints, each of the 5 vials were removed from heat, cooled to room temperature, and the solution was diluted with 500 μL of acetonitrile to precipitate proteins and nondesirable solutes. Each mixture was vortexed vigorously, passed through a 0.2 um Whatman Uniprep sample filter device, and analyzed by LC-MS (the methods of which are described in Example 12). The UV areas for the peak corresponding to PFT-α (retention time=1.8 minutes) and for the peak corresponding to PFT-β (retention time=3.9 minutes) were tabulated. The results are set forth in Table 3.

TABLE 3

| Human Serum | | | | | |
|---|---|---|---|---|---|
| Stability | 0 hours | 1.5 hours | 3 hours | 4.5 hours | 17 hours |
| PFT-α peak area | 547169 | 312351 | 156823 | 118842 | 7927 |
| (254 nm) | | | | | |
| PFT-β peak area | 13781 | 259004 | 448826 | 512198 | 696098 |
| (254 nm) | | | | | |

The method set forth above was repeated wherein 50 mMolar phosphate buffer (pH=7.4) was substituted for human serum. The results are provided in Table 4.

TABLE 4

| Phosphate Buffer | | | | | |
|---|---|---|---|---|---|
| pH = 7.4 Stability | 0 hours | 1.5 hours | 3 hours | 4.5 hours | 17 hours |
| PFT-α peak area | 584109 | 448284 | 303800 | 227004 | 18724 |
| (254 nm) | | | | | |
| PFT-β peak area | 13032 | 176201 | 307793 | 427152 | 601840 |
| (254 nm) | | | | | |

In both phosphate buffer and in human serum, PFT-α, at an initial concentration of 50 μMolar, converts rapidly to PFT-β with no other peaks detected by LC/MS. In phosphate buffer, half of PFT-α is converted to PFT-β in about 1.8 hours. In human serum, half of PFT-α is converted to PFT-β in about 3 hours. The slower conversion in human serum may be due to lipophilic interactions of PFT-α with albumin or other serum proteins which may give a stabilizing influence to the PFT-α form. It should be noted that the experimental conditions provided for complete recovery of the organic soluble components at each timepoint by providing a separate vial for each timepoint analyzed. This is significant because the aqueous solubility of cyclic PFT-β is extremely limited (about 3 μMolar). The rapid conversion of aqueous, soluble PFT-α to the very insoluble PFT-β has important ramifications in both biochemical investigations of pifithrin and in early stage drug development activities.

### EXAMPLE 16

This example demonstrates a method of producing an N-protected pifithrin derivative.

Compound (N) of FIG. 12 was generated using the following procedure. The compound was characterized using the methods set forth in Example 12. A solution of 100 mg PFT-α hydrobromide in 2 mL acetonitrile was treated with trifluoroacetic anhydride (1.1 equivalents) and triethylamine (2.1 equivalents). The mixture was stirred for 18 hours and the solvent was removed. Dichloromethane (8 mL) was added, and the solution was washed with water (5 mL). The organics were dried over sodium sulfate, and the solvent was removed to produce 113 mg of an off-white solid. The presence of compound (N) was confirmed by electrospray LC-MS and comprised the following characteristics: t_{R}=4.2 minutes, MS [M=C₁₈H₁₇F₃N₂O₂S] m/z 383 (MH⁺), 424 (MH—CH₃CN⁺).

### EXAMPLE 17

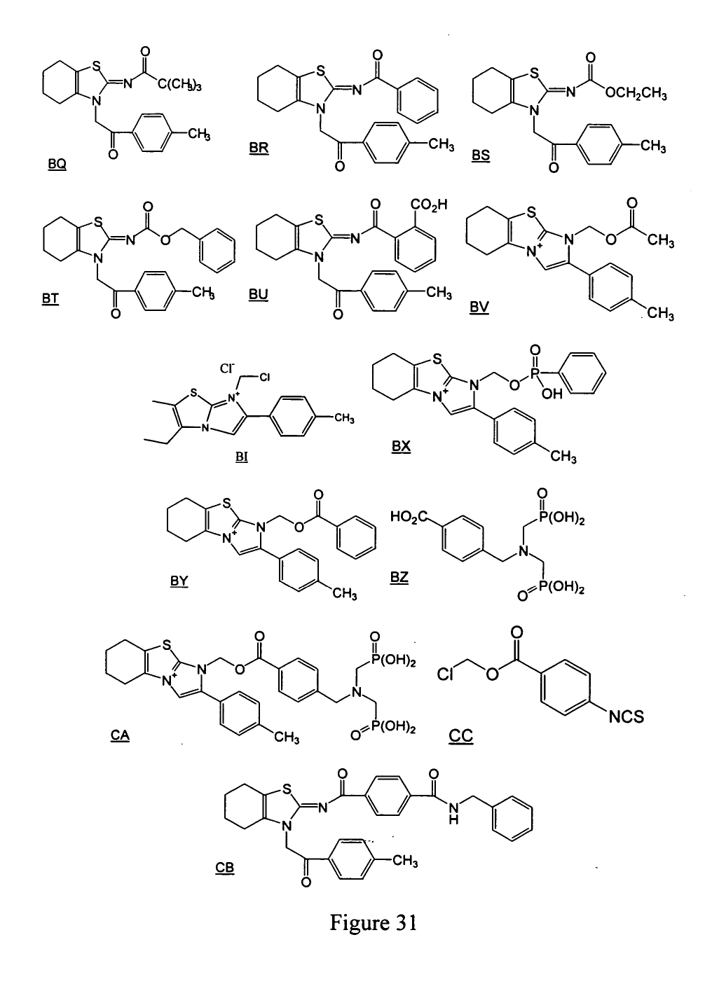

This example provides a method of generating compound (BR) of FIG. 31.

A solution of 100 mg PFT-A hydrobromide in 2 mL acetonitrile was treated with benzoyl chloride (1.1 equivalents) and triethylamine (2.1 equivalents). The mixture was stirred overnight, and the solvent was removed. Dichloromethane (8 mL) was added, and the solution was washed with water (5 mL). The organics were dried over sodium sulfate and the solvent was removed to provide 104 mg of an off-white solid. The presence of compound (BR) was confirmed by electrospray LC-MS and exhibited the following characteristics: t_{R}=4.3 minutes, MS [M=C₂₁H₂₆N₂O₂S] m/z 371(MH⁺).

### EXAMPLE 18

This example provides a method of generating compound (BQ) of FIG. 31.

A solution of 100 mg PFT-α hydrobromide in 2 mL acetonitrile was treated with trimethylacetyl chloride (1.1 equivalents) and triethylamine (2.1 equivalents). The mixture was stirred overnight, and the solvent was removed. Dichloromethane (8 mL) was added, and the solution was washed with water (5 mL). The organics were dried over sodium sulfate, and the solvent was removed to provide 149 mg of a tan solid. The presence of compound (BQ) was confirmed by electrospray LC-MS and exhibited the following characteristics: t_{R}=4.2 minutes, MS [M=C₂₃H₂₂N₂O₂S] m/z 391(MH⁺).

### EXAMPLE 19

This example provides a method of generating compound (BS) of FIG. 31.

A solution of 100 mg PFT-α hydrobromide in 2 mL acetonitrile was treated with ethyl chloroformate (1.1 equivalents) and triethylamine (2.1 equivalents). The mixture was stirred overnight, and the solvent was removed. Dichloromethane (8 mL) was added, and the solution was washed with water (5 mL). The organics were dried over sodium sulfate, and the solvent was removed to provide 101 mg of a brown solid. The presence of compound (BS) was confirmed by electrospray LC-MS and exhibited the following characteristics: t_{R}=3.8 minutes, MS [M=C₁₉H₂₂N₂O₃S] m/z 359(MH⁺), 381(MNa⁺), 422(MNa—CH₃CN⁺).

### EXAMPLE 20

This example provides a method of generating compound (BT) of FIG. 31.

A solution of 100 mg PFT-α hydrobromide in 2 mL acetonitrile was treated with benzyl chloroformate (1.1 equivalents) and triethylamine (2.1 equivalents). The mixture was stirred overnight, and the solvent was removed. Dichloromethane (8 mL) was added, and the solution was washed with water (5 mL). The organics were dried over sodium sulfate, and the solvent was removed to provide 112 mg of a yellow solid. The presence of (BT) was confirmed by electrospray LC-MS and exhibited the following characteristics: t_{R}=4.3 minutes, MS [M=C₂₄H₂₄N₂O₃S] m/z 421(MH⁺), 444(MNa⁺), 485(MNa—CH₃CN⁺).

### EXAMPLE 21

This example provides a method of generating compound (BU) of FIG. 31.

A solution of 100 mg PFT-α hydrobromide in 2 mL acetonitrile was treated with phthalic anhydride (1.1 equivalents) and triethylamine (2.1 equivalents). The mixture was stirred overnight, and the solvent was removed. Dichloromethane (8 mL) was added, and the solution was washed with water (5 mL). The organics were dried over sodium sulfate, and the solvent was removed to provide 144 mg of an oil. The presence of compound (BU) was confirmed by electrospray LC-MS and exhibited the following characteristics: t_{R}=3.5 minutes, MS [M=C₂₄H₂₂N₂O₄S] m/z 435(MH⁺), 457(MNa⁺), 498(MNa—CH₃CN+).

### EXAMPLE 22

This example provides a method of generating compound (BV) of FIG. 31.

A solution of 15 mg PFT-β in 2 mL dichloromethane was treated with bromomethyl acetate (5 equivalents). After 4 days, more bromomethyl acetate (2.5 equivalents) was added, and the solution was stirred for 1 week. The solvent was removed to provide 31.2 mg of a brown oil. The presence of compound (BV) was confirmed by electrospray LC-MS and exhibited the following characteristics: t_{R}=1.9 minutes, MS [M=C₁₉H₂₁N₂O₂S] m/z 341(M⁺).

### EXAMPLE 23

This example provides a method of generating compound (BI) (pifithrin-β-CH₂Cl) of FIG. 31.

A solution of 1.076 g PFT-β in 2 mL dioxane was treated with paraformaldehyde (5 equivalents) and thionyl chloride (5 equivalents). The solution was heated to 100° C. for 3 hours. The solution was filtered, and the solvent was removed by rotary evaporation at 70° C. Acetonitrile (30 mL) was added, the mixture was stirred for 20 minutes, and was filtered to remove excess paraformaldehyde. Rotary evaporation and drying in a desiccator provided 1.33 g of a tan solid. The presence of compound (BI) was confirmed by electrospray LC-MS and exhibited the following characteristics: t_{R}=2.2 minutes, MS [M=C₁₇H₁₈ClN₂S] m/z 317(M). The compound also was confirmed by proton NMR spectroscopy: ¹H (CDCl₃) δ: 7.74 (s, 1H), 7.56 (d, 2H, J 7.9 Hz), 7.37 (d, 2H, J 7.3 Hz), 6.33 (s, 2H), 2.90 (m, 4H), 2.45 (s, 3H), 2.03 (m, 4H).

### EXAMPLE 24

This example provides a method of generating compound (BX) of FIG. 31.

A solution of 30 mg pifithrin-β-CH₂Cl (prepared according to the procedure of Example 23, (BI)) in 300 μL acetonitrile was treated with phenyphosphonic acid (5 equivalents) and diisopropylethylamine (5 equivalents). The mixture was stirred overnight. The material was purified via LC to provide 20.9 mg of compound (BX). The presence of compound (BX) was confirmed by electrospray LC-MS and exhibited the following characteristics: t_{R}=2.3 minutes, MS [M=C₂₃H₂₄N₂O₃PS] m/z 439(M⁺).

### EXAMPLE 25

This example provides a method of generating compound (BY) of FIG. 31.

A solution of 40 mg pifithrin-β-CH₂Cl (prepared according to the procedure of Example 23) in 400 μL acetonitrile was treated with benzoic acid (3 equivalents) and diisopropylethylamine (3 equivalents). The mixture was stirred overnight. The material was purified via LC to provide 14.0 mg of compound (BY). The presence of compound (BY) was confirmed by electrospray LC-MS and exhibited the following characteristics: t_{R}=2.6 minutes, MS [M=C₂₄H₂₃N₂O₂S] m/z 403(M⁺).

### EXAMPLE 26

This example provides a method of generating compound (BZ) of FIG. 31.

A solution of 1.58 g phosphorus acid in 20 mL dioxane was treated with concentrated (12.1 M) hydrochloric acid (4.2 mL), 4-(aminomethyl)benzoic acid (1.0 g) and 37% (wt/wt) aqueous formaldehyde (1.42 mL). The mixture was stirred overnight at 105° C. and then cooled, resulting in a liquid containing a white solid. The solvent was removed via rotary evaporation at 80° C. to provide a slurry which was broken up with water (5 mL) and filtered. The solid was washed with water (5 mL) and dried under vacuum to give 1.66 g of a tan solid. The presence of compound (BZ) was confirmed by electrospray LC-MS and exhibited the following characteristics: t_{R}=0.6 minutes, MS [M=C₁₀H₁₅NO₈P₂] m/z 338(M−H⁻), 340(MH⁺).

### EXAMPLE 27

This example provides a method of generating compound (CA) of FIG. 31.

A solution of 185 mg pifithrin-β-CH₂Cl (prepared according to the procedure of Example 23) in 925 μL acetonitrile was treated with phosphonated carboxylic acid (prepared via the procedure of Example 26, 1.3 equivalents) and diisopropylethylamine (2.6 equivalents). The mixture was stirred overnight. The material was purified via LC to provide 30 mg of compound (CA). The presence of compound (CA) was confirmed by electrospray LC-MS and exhibited the following characteristics: t_{R}=2.1 minutes, MS [M=C₂₇H₃₂N₃O₈P₂S] m/z 620(M⁺), 618(M−2H⁻). The compound also was confirmed by proton NMR spectroscopy: ¹H (CDCl₃) δ: 8.01 (d, 2H, J 8.1 Hz), 7.82 (s, 1H), 7.67 (d, 2H, J 8.3 Hz), 7.46 (d, 2H, J 8.2 Hz), 7.35 (d, 2H, J 8.2 Hz), 6.18 (s, 2H), 4.65 (s, 4H), 3.18-3.12 (m, 6H), 2.70 (d, 4H, J 22.5 Hz), 2.33 (s, 3H), 1.84 (br s, 4H).

### EXAMPLE 28

This example provides a method of generating compound (CB) of FIG. 31.

A solution of 100 mg PFT-α hydrobromide in 1 mL acetonitrile was treated with terephthaloyl chloride (1.1 equivalents) and triethylamine (2.1 equivalents). After 1 hour, acetonitrile (1 mL) and benzylamine (2.1 equivalents) were added. The solution was stirred overnight. The presence of compound (CB) was confirmed by electrospray LC-MS and exhibited the following characteristics: t_{R}=4.3 minutes, MS [M=C₃₁H₂₉N₃O₃S] m/z 524(MH⁺), 522(M−H⁻).

### EXAMPLE 29

This example provides a method of generating compound (CC) of FIG. 31.

To 1.0 g of 4-carboxyphenyl isothiocyanate was added dichloromethane (15 mL) and distilled water (15 mL). The flask was cooled in an ice-water bath. Sodium bicarbonate (4.0 equivalents) and n-tetrabutylammonium hydrogen sulfate (0.05 equivalents) were added. After 10 min, chloromethyl chlorosulfate (1.2 equivalents) was added. The solution was stirred vigorously overnight and transferred to a separatory funnel with the aid of dichloromethane (10 mL). The layers were separated, and the organics were washed with saturated sodium chloride (20 mL). The organics were dried over sodium sulfate, and the solvent was removed to provide 1.10 g of a tan solid. The presence of compound (CC) was indicated by a shift in retention time in LS of the product (4.2 minutes) versus starting carboxylic acid (3.2 minutes). The compound also was confirmed by proton NMR spectroscopy: ¹H (CDCl₃) δ: 8.08 (d, 2H, J 8.8 Hz), 7.30 (d, 2H, J 8.8 Hz), 5.95 (s, 2H).

### EXAMPLE 30

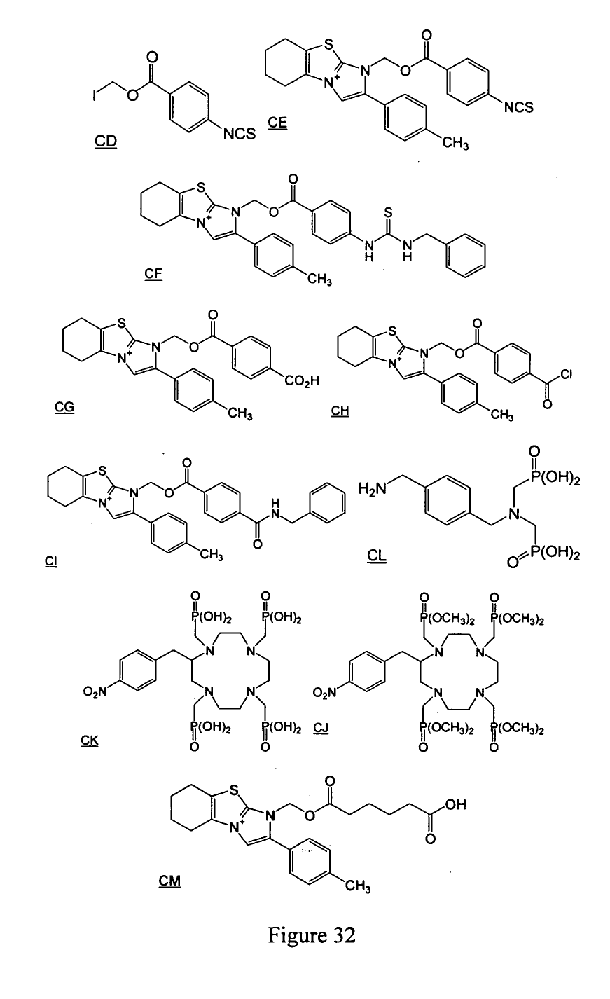

This example provides a method of generating compound (CD) of FIG. 32.

A solution of 250 mg of the chloromethyl ester prepared via the procedure of Example 29 in 2 mL acetone was treated with sodium iodide (1.2 equivalents). The solution was stirred overnight. The solution was filtered, the solvent removed, and the resulting residue was taken up in dichloromethane (10 mL). The solution was washed with 10% (w/v) sodium sulfite (10 mL), 5% (w/v) sodium bicarbonate (10 mL), and water (10 mL). The organics were dried over sodium sulfate, and the solvent was removed to provide 137 mg of a light green solid. The presence of compound (CD) was indicated by a shift in retention time in LS of the iodomethyl ester product (4.4 minutes) versus starting chloromethyl ester (4.2 minutes). The compound also was confirmed by proton NMR spectroscopy: ¹H (CDCl₃) δ: 8.04 (d, 2H, J 8.8 Hz), 7.29 (d, 2H, J 8.1 Hz), 6.15 (s, 2H).

### EXAMPLE 31

This example provides an alternate method of generating compound (CD) of FIG. 32.

A solution of 387 mg of the chloromethyl ester prepared via the procedure of Example 29 in 6 mL 2-butanone was treated with sodium iodide (1.2 equivalents). The solution was heated for 10 hours. The solution was filtered, the solvent removed, and the resulting residue was taken up in dichloromethane (10 mL). The solution was washed with 10% (w/v) sodium sulfite (10 mL), 5% (w/v) sodium bicarbonate (10 mL), and water (5 mL). The organics were dried over sodium sulfate, and the solvent was removed to provide 310 mg of a tan solid. The presence of compound (CD) was indicated by a shift in retention time in LS of the iodomethyl ester product (4.4 minutes) versus starting chloromethyl ester (4.2 minutes), in agreement with the results set forth in Example 30.

### EXAMPLE 32

This example provides a method of generating compound (CE) of FIG. 32.

A solution of 51.5 mg of pifithrin-β-CH₂Cl (prepared according to the procedure of Example 23) in 400 μL acetonitrile was treated with 4-carboxyphenyl-isothiocyanate (1.5 equivalents) and diisopropylethylamine (1.5 equivalents). The mixture was stirred overnight. The presence of compound (CE) was confirmed by electrospray LC-MS and exhibited the following characteristics: t_{R}=3.0 minutes, MS [M=C₂₅H₂₂N₃O₂S₂] m/z 460(M⁺).

Compound (CE) also was generated using an alternate method. A solution of 25 mg pifithrin-β in 1 mL dichloromethane was treated with the iodomethyl ester (prepared via the procedure described in Example 31, 1.5 equivalents). The mixture was stirred for 9 days. The presence of compound (CE) was confirmed by electrospray LC-MS and exhibited the following characteristics: t_{R}=3.0 minutes, MS [M=C₂₅H₂₂N₃O₂S₂] m/z 460(M⁺).

### EXAMPLE 33

This example provides a method of generating compound (CF) of FIG. 32.

The solution from Example 32 was treated with benzylamine (2.0 equivalents). The mixture was stirred overnight. The presence of compound (CF) was confirmed by electrospray LC-MS and exhibited the following characteristics: t_{R}=2.9 minutes, MS [M=C₃₂H₃₁N₄O₂S₂] m/z 567(M⁺).

### EXAMPLE 34

This example provides a method of generating compound (CG) of FIG. 32.

A solution of 60 mg of pifithrin-β-CH₂Cl (prepared according to the procedure of Example 23) in 1.2 mL acetonitrile was treated with terephthalic acid (1.1 equivalents) and diisopropylethylamine (2.2 equivalents). The mixture was stirred overnight. The solvent was removed to provide 78 mg of a brown oil. The presence of compound (CG) was confirmed by electrospray LC-MS and exhibited the following characteristics: t_{R}=2.4 minutes, MS [M=C₂₅H₂₃N₂O₄S] m/z 447(M⁺), 445([M−2H]⁻).

### EXAMPLE 35

This example provides a method of generating compound (CH) of FIG. 32.

A solution of 300 mg of pifithrin-β-CH₂Cl (prepared according to the procedure of Example 23) in 6 mL acetonitrile was treated with terephthalic acid (1.1 equivalents) and diisopropylethylamine (2.2 equivalents). The mixture was stirred overnight. The solvent was removed to produce a thick brown oil. The resulting oil was treated with ethyl acetate (10 mL), dichloromethane (13 mL), and 0.5 M hydrochloric acid (10 mL). The mixture was stirred for 10 min, and the liquid was removed. To the remaining solid was added dichloromethane (25 mL) and 2 M hydrochloric acid (10 mL). The mixture was stirred vigorously overnight. The layers were separated, and the organics were dried over sodium sulfate. The solvent was removed to provide 179 mg of the carboxylic acid. To 50 mg of this material in 1 mL of toluene was added thionyl chloride (34.0 equivalents), and the mixture was heated to 50-80° C. for two days. The solution was cooled, and the solvent was removed to provide a brown oil. The presence of compound (CH) was confirmed by electrospray LC-MS and exhibited the following characteristics: t_{R}=3.6 minutes, MS [M=C₂₅H₂₂ClN₂O₃S] m/z 465(M⁺).

### EXAMPLE 36

This example provides a method of generating compound (CI) of FIG. 32.

A solution of the acid chloride prepared in Example 35 in 1 mL dichloromethane was treated with diisopropylethylamine (1.05 equivalents) and benzylamine (1.05 equivalents). The mixture was stirred for two weeks. The solvent was removed to provide 72 mg of a brown solid. The presence of compound (CI) was confirmed by electrospray LC-MS and exhibited the following characteristics: t_{R}=2.7 minutes, MS [M=C₃₂H₃₀N₃O₃S] m/z 536(M⁺).

### EXAMPLE 37

This example provides a method of generating compound (CJ) of FIG. 32.

A solution of 64 mg of 2-p-nitrobenzyl-1,4,7,10-tetraazacyclododecane (Macrocyclics) in 500 μL dioxane was treated with paraformaldehyde (50 mg) and trimethylphosphite (207 mg). The mixture was heated to 85° C. The solvent was removed via rotary evaporation at 75° C. Chloroform (10 mL) was added, and the solution was washed with saturated sodium chloride (2×10 mL) and water (2×10 mL). The organics were dried over sodium sulfate, and the solvent was removed to provide a brown oil. The oil was purified via LC to provide the desired material. The presence of compound (CJ) was confirmed by electrospray LC-MS and exhibited the following characteristics: t_{R}=1.8 minutes, MS [M=C₂₇H₅₃N₅O₁₄P₄] m/z 796 (MH⁺), 818 (MNa⁺).

### EXAMPLE 38

This example provides a method of generating compound (CK) of FIG. 32.

A solution of 33 mg of the phosphonated macrocycle prepared in Example 37 in 700 μL dichloromethane was treated with bromotrimethylsilane (72 mg). The mixture was stirred overnight. Additional bromotrimethylsilane (36 mg) was added, and the resulting mixture was stirred an additional 3 days. Methanol (500 mL) was added, and the solution was stirred 1 hour. The volatiles were removed to give a brown oil. Addition of methanol precipitated a brown solid, which was filtered and dried. The resulting material was purified via LC to provide 2.7 mg of (CK). The presence of compound (CK) was confirmed by electrospray LC-MS and exhibited the following characteristics: t_{R}=1.5 minutes, MS [M=C₁₉H₃₇N₅O₁₄P₄] m/z 682 (M−H), 340 [(M−2H)/2)²⁻].

### EXAMPLE 39

This example provides a method of generating compound (CL) of FIG. 32.

A mixture of phosphorus acid (1.26 g), 6 M hydrochloric acid (19.5 mL), and p-xylenediamine (1.0 g) was heated to 100° C. To this was added 37% (wt/wt) aqueous formaldehyde (1.15 mL). The mixture was stirred at 100° C. overnight. The mixture was filtered, and the water was removed by rotary evaporation at 80° C. to provide 2.11 g of a white solid. The presence of compound (CL) was confirmed by electrospray LC-MS and exhibited the following characteristics: t_{R}=1.8 minutes, MS [M=C₁₀H₁₈N₂O₆P₂] m/z 325 (MH⁺).

### EXAMPLE 40

This example provides a method of generating compound (CM) of FIG. 32.

A solution of 164 mg of pifithrin-β-CH₂Cl (prepared according to the procedure of Example 23) in 2 mL acetonitrile was treated with adipic acid (1.1 equivalents, 75 mg) and diisopropylethylamine (2.2 equivalents, 132 mg). The mixture was stirred overnight. The presence of compound (CM) was confirmed by electrospray LC-MS and exhibited the following characteristics: t_{R}=2.3 minutes, MS [M=C₂₃H₂₇N₂O₄S] m/z 427(M⁺).

### EXAMPLE 41

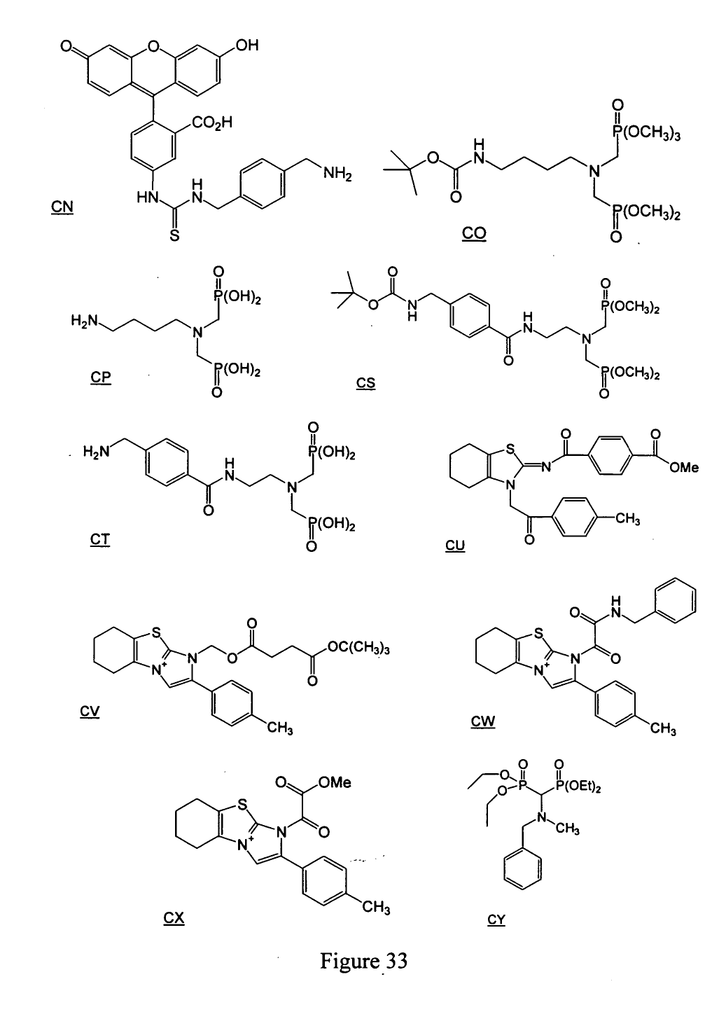

This example provides a method of generating compound (CN) of FIG. 33.

A solution of 210 mg p-xylylenediamine (3 equivalents) in 6 mL methanol was treated with FTIC, Isomer 1 (Calbiochem) in small portions over 1 hour. During the addition, a red-brown solid was produced. The mixture was stirred overnight, filtered, and washed with methanol (2 mL). The filtrate was concentrated to a red oil which solidified upon drying. A portion of this solid was purified via LC to provide 53 mg of an orange solid. The presence of compound (CN) was confirmed by electrospray LC-MS and exhibited the following characteristics: t_{R}=2.1 minutes, MS [M=C₂₉H₂₃N₃O₅S] m/z 526 (MH⁺).

### EXAMPLE 42

This example provides a method of generating compound (CO) of FIG. 33.

A solution of 928 mg N-BOC-1,4-diaminobutane in 10 mL dioxane was treated with paraformaldehyde (592 mg) and trimethylphosphite (2.44 g). The mixture was stirred at 108° C. overnight. The solvent was removed by rotary evaporation at 75° C. Chloroform (10 mL) was added, and the solution was washed with saturated sodium chloride (2×10 mL) and water (2×10 mL). The organics were dried over sodium sulfate, and the solvent was removed to provide 1.55 g of an oil. The presence of compound (CO) was confirmed by electrospray LC-MS and exhibited the following characteristics: t_{R}=2.4 minutes, MS [M=C₁₅H₃₄N₂O₈P₂] m/z 455(MNa⁺).

### EXAMPLE 43

This example provides a method of generating compound (CP) of FIG. 33.

A solution of 783 mg of the phosphonate prepared Example 42 in 18 mL dichloromethane was treated with bromotrimethylsilane (2.2 g). The solution was stirred overnight. Methanol (10 mL) was added, and the mixture was stirred for 2 hour. The volatiles were removed to provide 1.22 g of a yellow oil. The presence of compound (CP) was confirmed by electrospray LC-MS: MS for [M=C₆H₁₈N₂O₆P₂] m/z 275(M−H⁻) was observed in the void volume.

### EXAMPLE 44

This example provides a method of generating PFT-α in the free base form.

A mixture of 4.0 g pifithrin-α hydrobromide, 1 M NaOH (60 mL), and chloroform (60 mL) was stirred for 5 minutes, during which time the solid reacted and dissolved. The layers were separated, and the organics were dried over sodium sulfate for 10 minutes. The solvent was removed by rotary evaporation at 50° C. for 15 minutes. The material was dried under vacuum to provide 2.90 g of a tan solid. The presence of PFT-α was confirmed by electrospray LC-MS and exhibited the following characteristics: t_{R}=2.1 minutes, MS [M=C₁₆H₁₈N₂OS] m/z 287 (MH⁺).

### EXAMPLE 45

This example describes a method of generating compound (DN) of FIG. 36.

A 500 mg portion of compound (DJ) (produced in accordance with Example 67 and illustrated in FIG. 33) was dissolved in 5 mL of dry THF. The mixture was treated with 1.002 mL (10 equivalents) of ethylenediamine (EDA), and allowed to stir for 2 hours. The solution was decanted from the formed solid. The solvent and excess EDA were removed from the decanted solution by rotoevaporation under vacuum to yield 0.8728 g of white solid. The product (DN) was characterized by LCMS: retention time 1.608 minutes and desired M+H observed at 294 m/z.

### EXAMPLE 46

This example provides a method of generating compound (CS) in FIG. 33.

A solution of 872 mg of the amine prepared in Example 45 in 10 mL dioxane was treated with paraformaldehyde (535 mg) and trimethylphosphite (2.21 g). The mixture was heated at 100° C. overnight. The solvent was removed by rotary evaporation at 80° C. to give a brown solid. Chloroform (25 mL) was added, and the solution was washed with water (15 mL). The organics were dried over sodium sulfate, and the solvent was removed to provide 241 mg of a yellow semi-solid. The resulting solid was purified via LC to provide 58.8 mg of compound (CS). The presence of compound (CS) was confirmed by electrospray LC-MS and exhibited the following characteristics: t_{R}=2.6 minutes, MS [M=C₂₁H₃₇N₃O₉P₂] m/z 538 (MH⁺), 560 (MNa⁺).

### EXAMPLE 47

This example provides a method of generating compound (CT) in FIG. 33.

A solution of 54.6 mg of the phosphonate prepared in Example 46 in 1 mL dichloromethane was treated with bromotrimethylsilane (156 mg). The mixture was stirred overnight. Ethanol (0.5 mL) and water (3 drops) were added, and the mixture was stirred for 1 hour. The volatiles were removed, and the material was dried under vacuum. The resulting material was taken up in water (1 mL) and lyophilized to provide 59 mg of a tan solid. The presence of compound (CT) was confirmed by electrospray LC-MS and exhibited the following characteristics: t_{R}=0.4 minutes, MS [M=C₁₂H₂₁N₃O₇P₂] m/z 380 (M−H⁻), 382 (MH⁺), 404 (MNa⁺).

### EXAMPLE 48

This example provides a method of generating compound (CU) in FIG. 33.

A mixture of 50 mg pifithrin-α hydrobromide, mono-methyl terephthalate (1.0 equivalents), 1-[3-(dimethylamino)propyl]-3-ethylcarbodiimide hydrochloride (1.5 equivalents), 1-hydroxybenzotriazole (1.5 equivalents), and diisopropylethylamine (8.0 equivalents) in 2 mL dimethylformamide was stirred overnight. Water (5 mL) was added, producing a precipitate, which was filtered and washed with water (3×2 mL). The material was dried under vacuum to provide 47.7 mg of a yellow solid. The presence of compound (CU) was confirmed by electrospray LC-MS and exhibited the following characteristics: t_{R}=4.6 minutes, MS [M=C₂₅H₂₄N₂O₄S] m/z 447 (M−H⁻), 449 (MH⁺).

### EXAMPLE 49

This example provides a method of generating compound (CV) in FIG. 33.

A solution of 40 mg of pifithrin-β-CH₂Cl (prepared according to the procedure of Example 23) in 2 mL acetonitrile was treated with mono-tert-butyl succinate (3.0 equivalents) and diisopropylethylamine (3 equivalents). The mixture was stirred overnight. The solvent was removed, and the material was purified via LC to provide 22 mg of desired product. The presence of compound (CV) was confirmed by electrospray LC-MS and exhibited the following characteristics: t_{R}=4.3 minutes, MS [M=C₂₅H₃₁N₂O₄S] m/z 430(M⁺).

### EXAMPLE 50

This example provides a method of generating compound (CW) in FIG. 33.

A solution of 40 mg pifithrin-β in 800 μL acetonitrile was treated with oxalyl chloride (2.0 equivalents) and was stirred overnight. Three drops of this mixture was added to a vial containing benzylamine (1.5 μL) in acetonitrile (500 μL). The presence of compound (CW) was confirmed by electrospray LC-MS and exhibited the following characteristics: t_{R}=2.8 minutes, MS [M=C₂₅H₂₄N₃O₂S] m/z 455(M⁺).

### EXAMPLE 51

This example provides a method of generating compound (CX) in FIG. 33.

A solution of 40 mg pifithrin-β in 800 μL acetonitrile was treated with oxalyl chloride (2.0 equivalents) and was stirred overnight. Three drops of this mixture was added to a vial containing methanol (500 μL). The presence of compound (CX) was confirmed by electrospray LC-MS and exhibited the following characteristics: t_{R}=4.4 minutes, MS [M=C₁₉H₁₉N₂O₃S] m/z 355(M⁺), 396 (M−CH₃CN⁺).

### EXAMPLE 52

This example provides a method of generating compound (CY) in FIG. 33.

Compound (CY) was prepared via the procedure of Kantoci et al., Syn. Commun., 26(10), 2037 (1996). A mixture of N-benzyl-N-methylamine (20.0 g), diethylphosphite (70.7 g) and triethylorthoformate (29.3 g) was stirred under argon at reflux (150° C.) for 5 hours. Ethanol was removed via rotary evaporation at 70° C., and the mixture was again heated at reflux overnight. The solution was diluted with 600 mL chloroform, and was washed with 1 M sodium hydroxide (3×100 mL) and saturated sodium chloride (3×150 mL). The organics were dried over sodium sulfate, and the solvent was removed to provide 74.0 g of a light yellow oil. Ten grams of this material was subjected to silica gel column chromatography using 14:4:1 ethyl acetate:hexane: methanol as eluent. This provided 6.08 g of a clear oil. The presence of compound (CY) was confirmed by electrospray LC-MS and exhibited the following characteristics: t_{R}=3.4 minutes, MS [M=C₁₇H₃₁NO₆P₂] m/z 408(MH⁺), 430 (MNa⁺), 471 (MNa−CH₃CN⁺).

### EXAMPLE 53

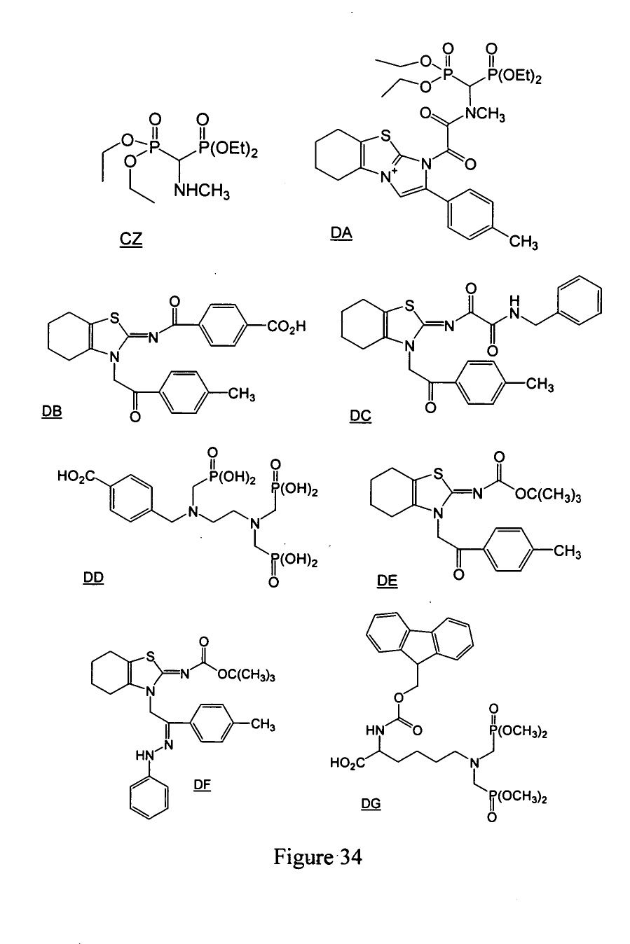

This example provides a method of generating compound (CZ) in FIG. 34.

Compound (CZ) (CAS # 80475-00-9) was prepared via the procedure of Kantoci et al., supra. A solution of 4.52 g of the phosphonated benzylamine prepared in Example 52 in methanol (45 mL) was treated with 10% palladium on carbon (200 mg) and subjected to an atmosphere of hydrogen overnight. The palladium/carbon was filtered to provide 2.98 g of a light yellow oil. The presence of compound (CZ) was confirmed by electrospray LC-MS and exhibited the following characteristics: t_{R}=1.9 minutes, MS [M=C₁₀H₂₅NO₆P₂] m/z 318(MH⁺).

### EXAMPLE 54

This example provides a method of generating compound (DA) in FIG. 34.

A solution of 40 mg pifithrin-β in 800 μL acetonitrile was treated with oxalyl chloride (2.0 equivalents) and was stirred overnight. To the solution was added the aminophosphonate prepared in Example 53 (3 equivalents). The presence of compound (DA) was confirmed by electrospray LC-MS and exhibited the following characteristics: t_{R}=4.0 minutes, MS [M=C₂₈H₄ON₃O₈P₂S] m/z 640(M⁺).

### EXAMPLE 55

This example provides a method of generating compound (DB) in FIG. 34.

A mixture of 100 mg pifithrin-α hydrobromide, terephthalic acid (1.0 equivalents), 1-[3-(dimethylamino)propyl]-3-ethylcarbodiimide hydrochloride (2.0 equivalents), 1-hydroxybenzotriazole (2.0 equivalents), and diisopropylethylamine (10.0 equivalents) in 4 mL dimethylformamide was stirred overnight. Water (11 mL) was added and the mixture was centrifuged, removing the solid. The resulting liquid was diluted in dichloromethane (30 mL) and 1 M hydrochloric acid (30 mL). The layers were separated, and the aqueous layer was extracted with fresh dichloromethane (30 mL). The organics were dried over sodium sulfate, and the residue was purified via LC to give 11.3 mg of a yellow solid. The presence of compound (DB) was confirmed by electrospray LC-MS and exhibited the following characteristics: t_{R}=4.0 minutes, MS [M=C₂₄H₂₂N₂O₄S] m/z 433 (M−H⁻), 435 (MH⁺).

### EXAMPLE 56

This example provides a method of generating compound (DC) in FIG. 34.

A solution of 50 mg pifithrin-α free base prepared in Example 44 in dichloromethane (2 mL) was treated with oxalyl chloride (1.5 equivalents). After 1 hour, a solution of benzylamine (4.0 equivalents) in dichloromethane (1 mL) was added. The mixture was stirred overnight. The presence of compound (DC) was confirmed by electrospray LC-MS and exhibited the following characteristics: t_{R}=4.1 minutes, MS [M=C₂₅H₂₅N₃O₃S] m/z 448 (MH⁺).

### EXAMPLE 57

This example describes a method of generating compound (DO) of FIG. 36.

A 500 mg sample of 4-chloromethylbenzoic acid (Aldrich) was dissolved in 8 mL of THF and treated all at once with 5 equivalents of ethylenediamine (Aldrich) (983 uL). After 24 hours, the solvent was removed under high vacuum. The resulting white solid (93%), compound (DO), was characterized by LCMS: retention time 0.4 minutes, desired M+H observed at 195 m/z.

### EXAMPLE 58

This example provides a method of generating compound (DD) in FIG. 34.

A mixture of 679 mg of the amino acid prepared in Example 57, 37% (wt/wt) aqueous formaldehyde (1.04 m]L), phosphorus acid (1.15 g), and concentrated (12.1 M) hydrochloric acid (2.3 mL) in dioxane (10 mL) was stirred at 100° C. overnight. The solvent was removed via rotary evaporation at 75° C. The mixture was centrifuged and the solid discarded. To the liquid was added more formaldehyde solution (1.04 mL), phosphorus acid (1.15 g), concentrated hydrochloric acid (2 mL), and dioxane (10 mL). The mixture was stirred overnight at 100° C. The solvent was removed via rotary evaporation at 75° C. to provide a thick oil. This was purified via LC to provide 276 mg of a brown solid. The presence of compound (DD) was confirmed by electrospray LC-MS and exhibited the following characteristics: t_{R}=0.6 minutes, MS [M=C₁₃H₂₃N₂O₁₁P₃] m/z 475 (M−H⁻), 477 (MH⁺).

### EXAMPLE 59

This example provides a method of generating compound (DE) in FIG. 34.

A solution of 500 mg pifithrin-α hydrobromide in acetonitrile (10 mL) was treated with di-tert-butyldicarbonate (1.1 equivalents) and triethylamine (2.1 equivalents). The mixture was stirred overnight, and the solvent was removed. Dichloromethane (20 mL) was added, and the solution was washed with water (10 mL). The organics were dried over sodium sulfate, and the solvent was removed to provide 488 mg of a tan solid. The presence of compound (DE) was confirmed by electrospray LC-MS and exhibited the following characteristics: t_{R}=4.5 minutes, MS [M=C₂₁H₂₆N₂O₃S] m/z 387 (MH⁺), 409 (MNa⁺), 450(MNa−CH₃CN⁺).

The BOC protecting group in compound DE allows for modification of the ketone functionality as illustrated by the preparation of compound DF in Example 62. Other conversions of the ketone functionality of compound DE can be accomplished using any conditions that do not remove the BOC protecting group (i.e. strong acids will remove the protecting group). After the conversion of the ketone into a reversible linker (as exemplified by DF in Example 62) the BOC protecting group can then be removed under acidic conditions (i.e. trifluoroacetic acid) or trimethylsilyl bromide to give pifithrin-alpha derivatives reversibly substituted only on the ketone group that are not readily obtainable by other methods.

### EXAMPLE 60

This example provides a method of generating compound (DF) in FIG. 34.

A solution of 100 mg of BOC-protected pifithrin-α prepared in Example 59 in acetonitrile (5 mL) was treated with phenylhydrazine (3 equivalents) and a catalytic amount of p-toluenesulfonic acid. The mixture was stirred overnight. Dichloromethane (20 mL) was added, and the solution was washed with water (20 mL). The organics were dried over sodium sulfate, and the solvent was removed to provide 131 mg of a brown oil. The presence of compound (DF) was confirmed by electrospray LC-MS and exhibited the following characteristics: t_{R}=5.8 minutes, MS [M=C₂₇H₃₂N₄O₂S] m/z 499 (MNa⁺), 540(MNa−CH₃CN⁺).

### EXAMPLE 61

This example provides a method of generating compound (DG) in FIG. 34.

A mixture of 6.0 g Fmoc-Lys-OH (Advanced ChemTech) in methanol (25 mL) and water (25 mL) was treated with 37% (wt/wt) aqueous formaldehyde (6.06 mL) and dimethylphosphite (8.96 g). The mixture was stirred at 80° C. for 2 hours, cooled, and extracted with dichloromethane (1×100 mL, 2×50 mL). The organics were washed with saturated sodium chloride (50 mL), dried over magnesium sulfate for 30 minutes, and the solvent was removed to provide 10.17 g of a light green oil. The presence of compound (DG) was confirmed by electrospray LC-MS and exhibited the following characteristics: t_{R}=3.3 minutes, MS [M=C₂₇H₃₈N₂O₁₀P₂] m/z 613 (MH⁺), 635 (MNa⁺).

### EXAMPLE 62

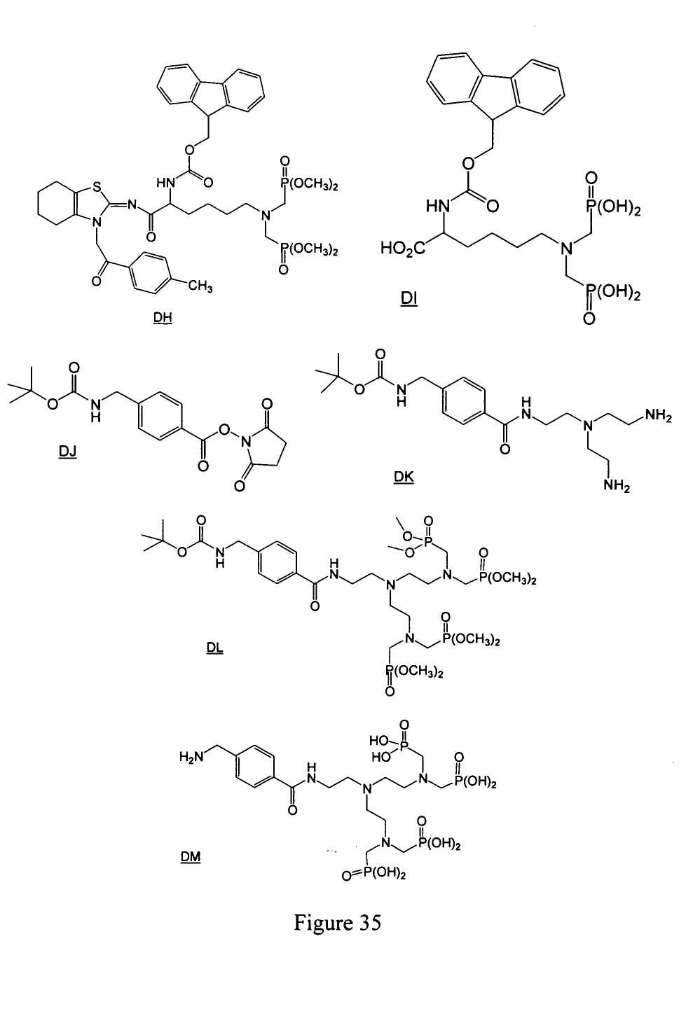

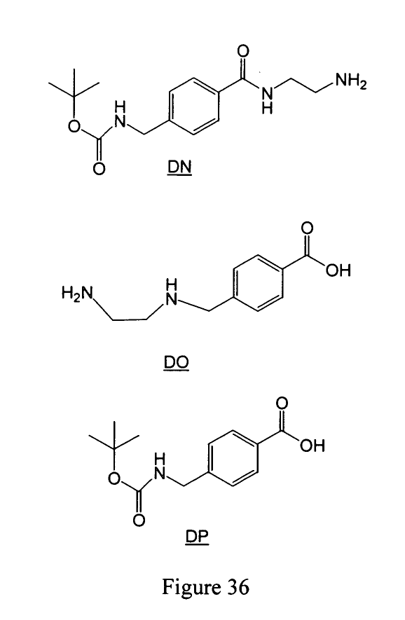

This example provides a method of generating compound (DH) in FIG. 35.

A mixture of 83 mg pifithrin-α free base prepared in Example 44, phosphonated Fmoc-Lys-OH prepared in Example 61 (1.1 equivalents), 1-[3-(dimethylamino)propyl]-3-ethylcarbodiimide hydrochloride (1.5 equivalents), 1-hydroxybenzotriazole (1.5 equivalents), and diisopropylethylamine (6.0 equivalents) in 2 mL dimethylformamide was stirred overnight. The presence of compound (DH) was confirmed by electrospray LC-MS; t_{R}=4.4 minutes, MS [M=C₄₃H₅₄N₄O₁₀P₂S] m/z 881 (MH⁺), 903 (MNa⁺).

### EXAMPLE 63

This example provides a method of generating compound (DI) in FIG. 35.

A solution of 23.9 mg phosphonated Fmoc-Lys-OH prepared in Example 61 in dichloromethane (1 mL) was treated with bromotrimethylsilane (60 mg). The mixture was stirred overnight. The presence of compound (DI) was confirmed by electrospray LC-MS and exhibited the following characteristics: t_{R}=3.4 minutes, MS [M=C₂₃H₃₀N₂O₁₀P₂] m/z 555 (M−H⁻).

### EXAMPLE 64

This example provides an alternate method of generating compound (DI) in FIG. 35.

A solution of 112.9 mg phosphonated Fmoc-Lys-OH prepared in Example 61 in 6 M hydrochloric acid (3 mL) was stirred at 80° C. for 2 days. Water (9 mL) was added. After 2 more days, the mixture was centrifuged and the liquid decanted. The solid was dried under vacuum to provide 86.8 mg of an off-white solid. The presence of compound (DI) was confirmed by electrospray LC-MS and exhibited the following characteristics: t_{R}=3.1 minutes, MS [M=C₂₃H₃₀N₂O₁₀P₂] m/z 555 (M−H⁻).

### EXAMPLE 65

This example provides an alternate method of generating compound (DI) in FIG. 35.

A solution of 500 mg Fmoc-Lys-OH (Advanced ChemTech) in dioxane (5 mL) was treated with 37% (wt/wt) aqueous formaldehyde (303 μL), phosphorus acid (333 mg), and concentrated (12.1 M) hydrochloric acid (674 μL). The mixture was stirred at 90° C. overnight, and the solvent was removed via rotary evaporation at 75° C. The presence of compound (DI) was confirmed by electrospray LC-MS; t_{R}=5.4 minutes, MS [M=C₂₃H₃₀N₂O₁₀P₂] m/z 555 (M−H⁻).

### EXAMPLE 66

The example provides a method of producing compound (DJ) of FIG. 36.

A 1.5 g portion of compound (DP) of Example 71 was dissolved in 17 mL of dry THF along with 0.69 g of N-hydroxysuccinimide (Aldrich). The mixture was treated all at once with 6 mL of 1M dicyclohexylcarbodiimide in dichloromethane with stirring. After 2 days, a white precipitate (dicyclohexylurea) was filtered. The filtrate was rotoevaporated under vacuum to yield 2.8146 g of white solid, which was characterized by LCMS: retention time 3.299 minutes and desired M+H observed at 349 m/z.

### EXAMPLE 67

This example provides a method of generating compound (DJ) in FIG. 35.

A solution of 1.29 g of the Boc-protected amino acid prepared in Example 71 in 15 mL tetrahydrofuran was treated with N-hydroxysuccinimde (623 mg) and 1.0 M 1,3-dicyclohexylcarbodiimide in dichloromethane (5.4 mL). The mixture was stirred overnight. A white precipitate was filtered, and the supernatant was concentrated to provide 1.89 g of a white solid. The presence of compound (DJ) was indicated by the presence of a UV signal associated with the peak at 3.3 minutes.

### EXAMPLE 68

This example provides a method of generating compound (DK) in FIG. 35.

To a solution of 2.1 g tris-(2-aminoethyl)amine in 20 mL tetrahydrofuran, a solution of 1.0 g of the activated ester prepared as in Example 66 in 20 mL tetrahydrofuran was added drop-wise over a period of 40 minutes. The mixture was stirred overnight resulting in a precipitate that was filtered and concentrated via rotary evaporation to provide 2.10 g of a yellow oil. The presence of compound (DK) was confirmed by electrospray LC-MS and exhibited the following characteristics: t_{R}=1.4 minutes, MS [M=C₁₉H₃₃N₅O₃] m/z 380 (MH⁺), 402 (MNa⁺).

### EXAMPLE 69

This example provides a method of generating compound (DL) in FIG. 35.

A solution of 2.08 g of the amine prepared in Example 68 in dioxane (20 mL) was treated with paraformaldehyde (1.50 g) and dimethylphosphite (6.85 g). The mixture was stirred at 90° C. overnight, and the solvent was removed via rotary evaporation at 70° C. Dichloromethane (50 mL) was added, and the mixture was washed with saturated sodium chloride (25 mL) and water (25 mL). The organics were dried over sodium sulfate, and the solvent was removed. The residue was purified via LC to provide 123.8 mg of a yellow oil. The presence of compound (DL) was confirmed by electrospray LC-MS and exhibited the following characteristics: t_{R}=2.2 minutes, MS [M=C₃₁H₆₁N₅O₁₅P₄] m/z 868 (MH⁺).

### EXAMPLE 70

This example provides a method of generating compound (DM) in FIG. 35.

A solution of 111.11 mg of the phosphonated diamine prepared in Example 69 in 1 mL dichloromethane was treated with 194 mg of bromotrimethylsilane. After 5 hours, methanol (1 mL) was added. The mixture was stirred for 1 hour, and the solvent was removed to provide 113.9 mg of a tan solid. The presence of compound (DM) was confirmed by electrospray LC-MS and exhibited the following characteristics; t_{R}=1.0 minutes, MS [M=C₁₈H₃₇N₅O₁₃P₄] m/z 328 [(M+2H/2)²⁺)], 656 (MH⁺). The compound also was analyzed by proton NMR spectroscopy: ¹H (CDCl₃) δ: 7.77 (d, 2H, J 8.1 Hz), 7.43 (d, 2H, J 8.2 Hz), 4.1-3.3 (m, 33H).

### EXAMPLE 71

This example provides a method of generating compound (DP) in FIG. 36.

A 2.0 g portion of 4-aminomethyl benzoic acid (Aldrich) was dissolved in 20 mL of water containing 0.64 g of solid NaOH. A 3.18 g portion of Boc anhydride (Aldrich) was added, and the resulting mixture was stirred overnight. The mixture was adjusted to pH=2 by the careful addition of 15 mL of 2N HCl. The resulting white solid was filtered and dried to yield 2.9997 g of product (DP). The compound (DP) was characterized by LCMS: retention time 2.901 minutes, desired M-H mass ion observed at 250 m/z.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inhibiting cell death in a mammal, wherein the method comprises administering to a mammal an effective amount of a composition comprising a cell protection factor covalently linked to a bone targeting agent via a linkage that is cleaved under physiological conditions, whereby the cell protection factor is released from the bone targeting agent in vivo to inhibit cell death.

2. The method of claim 1, wherein the cell protection factor is a temporary p53 inhibitor.

3. The method of claim 2, wherein the cell protection factor is a compound of Formula I: 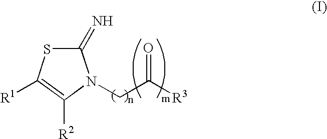
wherein m is 0 or 1, n is an integer from 1 to 4,
R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and optionally forms a C₃-C₆ cycloalkyl when R³ is connected to the carbon alpha to the thiazole ring.

4. The method of claim 3, wherein m is 0, n is 2, and R³ is a one-carbon alkyl such that the three-carbon chain forms a cyclopropyl group, whereby the cell protection factor is a compound of Formula X: 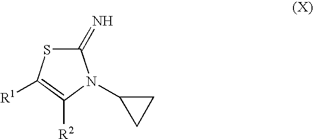
wherein R¹ and R² are taken together to form an aliphatic or aromatic carbocyclic 5- to 8-membered ring optionally substituted with one or more straight or branched C₁-C₆ alkyl, C₁-C₆ alkoxy, fluoro, chloro, bromo, nitro, amino, C₁-C₆ alkylamino, and/or C₄-C₁₄ aromatic or heteroaromatic moieties.

5. The method of claim 3, wherein R¹ and R² are taken together to form a 5- or 6-membered aliphatic carbocyclic ring optionally substituted with one or more C₁-C₆ alkyl groups.

6. The method of claim 2, wherein the cell protection factor is a compound of Formula IV: 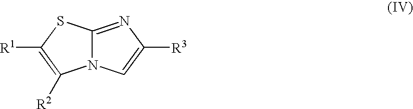
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties.

7. The method of claim 6, wherein R¹ and R² are taken together to form a 5- or 6-membered aliphatic carbocyclic ring optionally substituted with one or more C₁-C₆ alkyl groups.

8. The method of claim 5, wherein the cell protection factor is a compound of Formula II: 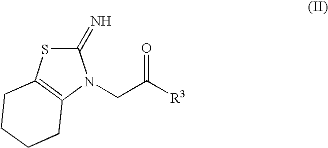
wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic groups.

9. The method of claim 8, wherein the cell protection factor is a compound of Formula III: 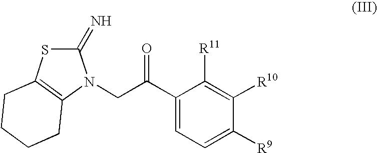
wherein R⁹, R¹⁰, and R¹¹ are each independently a hydro, methyl, fluoro, chloro, bromo, nitro, amino, methoxy, or phenyl moiety.

10. The method of claim 9, wherein the cell protection factor is 2-[2-imino-4,5,6,7-tetrahydro-1,3-benzothiazol-3(2H)-yl]-1-(4-methylphenyl)-1-ethanone or 2-[2-imino-4,5,6,7-tetrahydro-1,3-benzothiazol-3(2H)-yl]-1-(biphenyl)-1-ethanone.

11. The method of claim 1, wherein the inhibited cell death is bone marrow cell death.

12. The method of claim 11, wherein the cell death is caused by exposure to at least one chemical or radiation.

13. The method of claim 6, wherein the inhibited cell death is bone marrow cell death.

14. The method of claim 13, wherein the cell death is caused by exposure to at least one chemical or radiation.

15. The method of claim 9, wherein the inhibited cell death is bone marrow cell death.

16. The method of claim 15, wherein the cell death is caused by exposure to at least one chemical or radiation.

17. The method of claim 1, wherein the mammal comprises at least one tumor.

18. The method of claim 17, wherein the mammal comprises at least one p53+tumor.

19. The method of claim 6, wherein the mammal comprises at least one tumor.

20. The method of claim 19, wherein the mammal comprises at least one p53+tumor.

21. The method of claim 9, wherein the mammal comprises at least one tumor.

22. The method of claim 21, wherein the mammal comprises at least one p53+tumor.

23. The method of claim 1, wherein the bone targeting agent is selected from the group consisting of a bisphosphonate, a hydroxybisphosphonate, a phosphonate, a phosphate, an aminomethylenephosphonic acid, and an acidic peptide.

24. The method of claim 3, wherein the bone targeting agent is selected from the group consisting of a bisphosphonate, a hydroxybisphosphonate, a phosphonate, a phosphate, an aminomethylenephosphonic acid, and an acidic peptide.

25. The method of claim 6, wherein the bone targeting agent is selected from the group consisting of a bisphosphonate, a hydroxybisphosphonate, a phosphonate, a phosphate, an aminomethylenephosphonic acid, and an acidic peptide.

26. The method of claim 9, wherein the bone targeting agent is selected from the group consisting of a bisphosphonate, a hydroxybisphosphonate, a phosphonate, a phosphate, an aminomethylenephosphonic acid, and an acidic peptide.

27. The method of claim 1, wherein the linker is an acid-cleavable linker.

28. The method of claim 3, wherein the linker is an acid-cleavable linker.

29. The method of claim 6, wherein the linker is an acid-cleavable linker.

30. The method of claim 9, wherein the linker is an acid-cleavable linker.

31. The method of claim 27, wherein the linker is an enol ether, ketal, imine, oxime, hydrazone, semicarbazone, acylimide, or methylene radical.

32. The method of claim 28, wherein the linker is an enol ether, ketal, imine, oxime, hydrazone, semicarbazone, acylimide, or methylene radical.

33. The method of claim 29, wherein the linker is an enol ether, ketal, imine, oxime, hydrazone, semicarbazone, acylimide, or methylene radical.

34. The method of claim 30, wherein the linker is an enol ether, ketal, imine, oxime, hydrazone, semicarbazone, acylimide, or methylene radical.

35. The method of claim 1, wherein the linker is a hydrolytically cleavable linker.

36. The method of claim 1, wherein the linker is cleaved enzymatically.

37. The method of claim 1, wherein the mammal is a human.

38. A compound of Formula V: 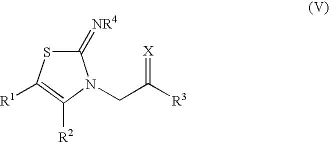
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
R⁴ is hydrogen or an C₁-C₆ acyl group when X is Q, or R⁴ is Q when X is a carbonyl or protected carbonyl, and
X is Q, a carbonyl, or a protected carbonyl,
wherein Q is an organic moiety that contains a nucleophilic or electrophilic reacting group and is cleavable under physiological conditions, thereby releasing a temporary p53 inhibitor.

39. The compound of claim 38, wherein Q is an organic moiety that is cleavable under acidic physiological conditions.

40. The compound of claim 38, wherein Q is an organic moiety that is hydrolytically cleavable under physiological conditions.

41 The compound of claim 38, wherein Q is an enol ether, ketal, imine, oxime, hydrazone, semicarbazone, acylimide, or methylene radical.

42. The compound of claim 38, wherein Q is an organic moiety that is enzymatically cleavable.

43. The compound of claim 38, wherein Q is A-J, wherein A is an organic moiety that is cleavable under physiological conditions, and J is a bone targeting agent.

44. A compound of Formula VI: 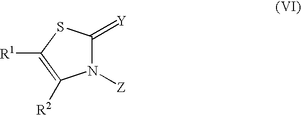
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
Y and Z taken together complete a 5-member imidazole ring of Formula VII or Formula VIII, 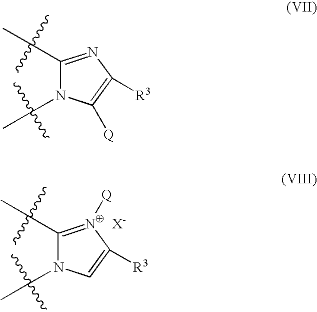
wherein X⁻ is selected from the group consisting of a chloride, a bromide, a fluoride, an iodide, an acetate, a formate, a phosphate, a sulfate, and other pharmaceutically acceptable anions, and Q is an organic moiety that contains a nucleophilic or electrophilic reacting group and is cleavable under physiological conditions.

45. The compound of claim 44, wherein Q is an organic moiety that is cleavable under acidic physiological conditions.

46. The compound of claim 44, wherein Q is an organic moiety that is hydrolytically cleavable under physiological conditions.

47. The compound of claim 44, wherein Q is an enol ether, ketal, imine, oxime, hydrazone, semicarbazone, acylimide, or methylene radical.

48. The compound of claim 44, wherein Q is an organic moiety that is enzymatically cleavable.

49. The compound of claim 44, wherein Q is A-J, wherein A is an organic moiety that is cleavable under physiological conditions, and J is a bone targeting agent.

50. A compound of Formula V: 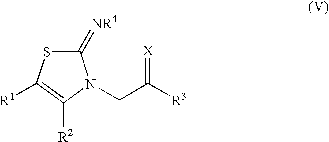
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
X is A-J, a carbonyl, or a protected carbonyl, and
R⁴ is hydrogen or an C₁-C₆ acyl group when X is A-J or R⁴ is A-J when X is a carbonyl or protected carbonyl,
wherein A is an organic moiety that is cleavable under physiological conditions, and J is a bone targeting agent.

51. The compound of claim 50, wherein A is an organic moiety that is cleavable under acidic physiological conditions.

52. The compound of claim 50, wherein A is an organic moiety that is hydrolytically cleavable under physiological conditions.

53. The compound of claim 50, wherein A is an organic moiety that is enzymatically cleavable.

54. The compound of claim 50, wherein the bone targeting agent is selected from the group consisting of a bisphosphonate, a hydroxybisphosphonate, a phosphonate, a phosphate, an aminomethylenephosphonic acid, and an acidic peptide.

55. A compound of Formula VI: 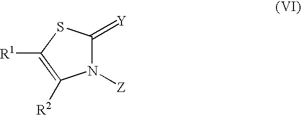
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and Y and Z taken together complete a 5-member imidazole ring of Formula VII or Formula VIII, 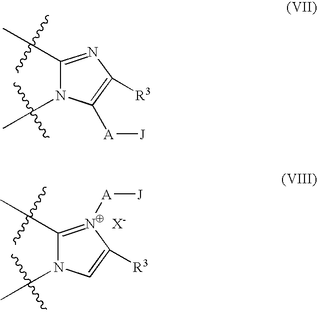
wherein R³ is selected from the group consisting of a C₁-C₆ alkyl group, a C₁-C₆ alkoxy group, and a phenyl group, wherein the alkyl group, the alkoxy group, or the phenyl group is optionally substituted with one or more straight or branched C₁-C₆ alkyls, C₁-C₆ alkoxy, hydroxy, fluoro, chloro, bromo, nitro, amino, C₁-C₆ alkylamino, and/or C₄-C₁₄ aromatic or heteroaromatic moieties, X⁻ is selected from the group consisting of a chloride, a bromide, a fluoride, an iodide, an acetate, a formate, a phosphate, a sulfate, and other pharmaceutically acceptable anions, and
A is an organic moiety that is cleavable under physiological conditions, and J is a bone targeting agent.

56. The compound of claim 55, wherein A is an organic moiety that is cleavable under acidic physiological conditions.

57. The compound of claim 55, wherein A is an organic moiety that is hydrolytically cleavable under physiological conditions.

58. The compound of claim 55, wherein A is an organic moiety that is enzymatically cleavable.

59. The compound of claim 55, wherein the bone targeting agent is selected from the group consisting of a bisphosphonate, a hydroxybisphosphonate, a phosphonate, a phosphate, an aminomethylenephosphonic acid, and an acidic peptide.

60. A compound of Formula IX: 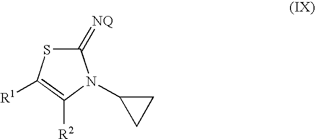
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties,
wherein Q is an organic moiety that contains a nucleophilic or electrophilic reacting group and is cleavable under physiological conditions.

61. The compound of claim 60, wherein Q is an organic moiety that is cleavable under acidic physiological conditions.

62. The compound of claim 60, wherein Q is an organic moiety that is hydrolytically cleavable under physiological conditions.

63. The compound of claim 60, wherein Q is an organic moiety that is enzymatically cleavable.

64. The compound of claim 60, wherein Q is A-J, wherein A is an organic moiety that is cleavable under physiological conditions and J is a bone targeting agent.

65. The compound of claim 44, wherein Q in Formula VIII is —CH₂O—.

66. The compound of claim 65, wherein the compound is Formula XI: 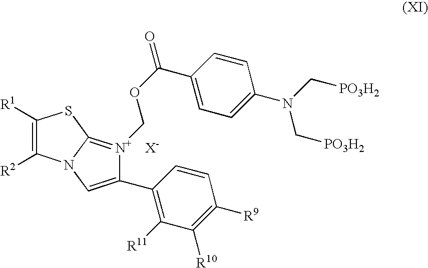
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, X⁻ is selected from the group consisting of a chloride, a bromide, a fluoride, an iodide, an acetate, a formate, a phosphate, a sulfate, and other pharmaceutically acceptable anions, and
R⁹, R¹⁰, and R¹¹ are each independently a hydro, methyl, fluoro, chloro, bromo, nitro, amino, methoxy, or phenyl moiety.

67. The compound of claim 66, wherein the compound is Formula XII: 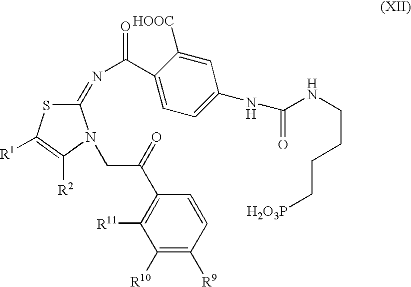
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R⁹, R¹⁰, and R¹¹ are each independently a hydro, methyl, fluoro, chloro, bromo, nitro, amino, methoxy, or phenyl moiety.

68. The compound of claim 55, wherein A of Formula VIII is —CH₂O— and J is a bone targeting agent.

69. The compound of claim 68, wherein the compound is Formula XI: 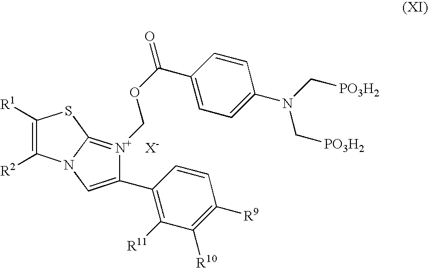
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, X⁻ is selected from the group consisting of a chloride, a bromide, a fluoride, an iodide, an acetate, a formate, a phosphate, a sulfate, and other pharmaceutically acceptable anions, and
R⁹, R¹⁰, and R¹¹ are each independently a hydro, methyl, fluoro, chloro, bromo, nitro, amino, methoxy, or phenyl moiety.

70. The compound of claim 69, wherein the compound is Formula XII: 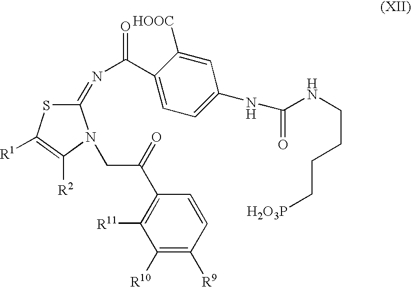
wherein R¹ and R² are taken together to form an aliphatic or aromatic carbocyclic 5- to 8-membered ring, optionally substituted with one or more straight or branched C₁-C₆ alkyl, C₁-C₆ alkoxy, hydroxy, fluoro, chloro, bromo, nitro, amino, C₁-C₆ alkylamino, and/or C₄-C₁₄ aromatic or heteroaromatic moieties, and
R⁹, R¹⁰, and R¹¹ are each independently a hydro, methyl, fluoro, chloro, bromo, nitro, amino, methoxy, or phenyl moiety.

71. The compound of claim 38, wherein X is a carbonyl and R⁴ is Q or A, wherein Q is an acid cleavable group, and wherein A selected from the group consisting of 4-aminophthalic acid, succinic acid, 4-aminophenylacetic acid, and 4-aminobenzoic acid.

72. The compound of claim 43, where the bone targeting agent is selected from a group consisting of alendronate, pamidronate, 4-aminobutylphosphonic acid, N,N,N,N-tetrakis-(phosphonomethyl)-ethylenediamine, 1-hydroxyethane-1,1-diphosphonic acid, phytic acid, N,N,N,N-tetrakis(methylphosphono)-1,5,8,12-tetraazacyclotetradecane, N,N-bis(methylphosphono)-4-amino-benzoic acid, nitrilotri(methylphosphonic acid), aspartyl hexapeptide, and glutamyl hexapeptide.

73. The compound of claim 50, where the bone targeting agent is selected from a group consisting of alendronate, pamidronate, 4-aminobutylphosphonic acid, N,N,N,N-tetrakis-(phosphonomethyl)-ethylenediamine, 1-hydroxyethane-1,1-diphosphonic acid, phytic acid, N,N,N,N-tetrakis(methylphosphono)-1,5,8,12-tetraazacyclotetradecane, N,N-bis(methylphosphono)-4-amino-benzoic acid, nitrilotri(methylphosphonic acid), aspartyl hexapeptide, and glutamyl hexapeptide.

74. The compound of claim 55, where the bone targeting agent is selected from a group consisting of alendronate, pamidronate, 4-aminobutylphosphonic acid, N,N,N,N-tetrakis-(phosphonomethyl)-ethylenediamine, 1-hydroxyethane-1,1-diphosphonic acid, phytic acid, N,N,N,N-tetrakis(methylphosphono)-1,5,8,12-tetraazacyclotetradecane, N,N-bis(methylphosphono)-4-amino-benzoic acid, nitrilotri(methylphosphonic acid), aspartyl hexapeptide, and glutamyl hexapeptide.
